(19) United States
(12) Patent Application Publication
(10) Pub. No.: US 2025/0302959 A1
(43) Pub. Date: Oct. 2, 2025

(54) ARTIFICIAL IMMUNE RECEPTORS

(71) Applicants: LEIBNIZ-INSTITUT FÜR IMMUNTHERAPIE (LIT), Regensburg (DE); Universität Regensburg, Regensburg (DE)

(72) Inventors: Thomas HEHLGANS, Wenzenbach (DE); Sebastian BITTNER, Roth (DE); Markus FEUERER, Regensburg (DE)

(21) Appl. No.: 18/864,985

(22) PCT Filed: May 22, 2023
(86) PCT No.: PCT/EP2023/063632

§ 371 Date: Nov. 12, 2024

(30) Foreign Application Priority Data
May 24, 2022 (EP) 22175091.2

(51) Int. Cl. A61K 40/41; A61K 40/11; A61K 40/31; A61P 37/06; C07K 14/705; C07K 14/715; C07K 14/725

(52) U.S. Cl. CPC A61K 40/416; A61K 40/11; A61K 40/31; A61P 37/06; C07K 14/7051; C07K 14/70578; C07K 14/7151; A61K 2239/22; A61K 2239/28; C07K 2319/03

(57) ABSTRACT

The present disclosure provides novel polypeptides, so called artificial immune receptors or AlRs, that are novel tools for cellular therapies. AIR-expressing regulatory T cells sense these environmental signals and translate them into a Treg TCR-like activating program, enabling Treg cells to fulfill their suppressive and tissue protective functions, independent of a specific TCR- or CAR-antigen and independent of endogenous TCR-MHC restriction.

### BACKGROUND

Regulatory T cells (Tregs) are a pivotal T cell population with various functions in the body. Tregs foster tolerance against self-antigens, allergens and commensals, thereby limiting self-reactivity of immune cells and excessive inflammation. Mutations in the Treg master transcription factor Foxp3 lead to lethal multiorgan autoinflammation, both in mice and humans (Nat Genet, 2001. 27(1): 18-20; Nat Genet, 2001. 27(1): 68-73). In the last years, it became evident that Treg exert additional functions in safeguarding tissue homeostasis and tissue regeneration (Immunology, 2020.161(1):4-17). On basis of these facts, Tregs can be considered as promising living drugs for autoimmune disorders and clinical trials have already proven the safety and efficacy of Treg-based cellular therapies (Front Immunol, 2019. 10: 43; Science, 2018. 362(6411): 154-155.

Innovative concepts applying the Chimeric Antigen Receptor (CAR) technology to Tregs are now the next step to enhance the potency of adoptive Treg cell therapy (Nat Rev Drug Discov, 2019. 18(10): 749-769.). Preclinical studies have already demonstrated the superiority of engineered Tregs with a CAR guided (auto-) antigen-specificity over Tregs with only a natural polyclonal TCR repertoire in diminishing alloimmune reactions in graft versus host disease (GvHD) and graft rejection after transplantation (Sci Transl Med, 2020. 12(557); Am J Transplant, 2020. 20(6): 1562-1573; J Clin Invest, 2016. 126(4): 1413-24; Am J Transplant, 2017. 17(4): 917-930; Am J Transplant, 2017. 17(4): p. 931-943). CAR-Tregs were also effectively used for treatment of asthma, hemophilia A, Type 1 diabetes, experimental autoimmune encephalitis (EAE) and inflammatory bowel disease (IBD) in preclinical models (J Neuroinflammation, 2012. 9: 112; J Immunol Methods, 2021. 488: 112931; Mol Ther, 2014. 22(5): 1018-28; Front Immunol, 2017. 8: p. 1125; J Autoimmun, 2019. 103: p. 102289).

However, in human autoimmune diseases the implicated autoantigens, which could serve as potential targets for CAR-Tregs, are in many cases not known, or multiple organs and tissues are affected without a uniform antigen. In contrast, the mediators of inflammation show a high redundancy in many inflammatory diseases, as well as a functional importance for the development of such diseases. Especially, cytokines of the Tumor-Necrosis-Factor (TNF) family are involved in many different inflammatory and autoimmune diseases, and therapeutic intervention of TNF receptor (TNFR) activation is an important treatment option for several inflammatory diseases (Trends Immunol, 2012. 33(3): 144-52). These considerations directed us to develop a new concept for engineered Treg cell therapy and to generate artificial immune receptors (AIRs) that target these inflammatory mediators instead of tissue specific antigens.

We focused on targets of the TNF family and chose receptors of the ligands LIGHT and Lα1β2, TNFα and TL1A, as these cytokines have pleiotropic roles in numerous autoimmune disease. Preclinical disease models and patient data indicate that LIGHT and Lα1β2 signaling through corresponding receptors HVEM and LTBR enhance pathology in IBD, autoimmune hepatitis, asthma, rheumatoid arthritis, multiple sclerosis and GvHD (reviewed in Immunol Rev, 2008. 223: 202-20.). A Role for TL1A and its receptor DR3 has been described in a variety of inflammatory diseases. Studies with blocking antibodies or TL1A- and DR3-deficient mice demonstrated a critical involvement for this receptor-ligand system in induction and maintenance of chronic inflammation in IBD, arthritis and EAE. Moreover, patient data and genome wide association studies point to a fundamental impact of TL1A-DR3 in human disease (reviewed in FEBS Lett, 2017. 591(17): 2543-2555.). The most prominent member of the TNF family, TNFα itself, has been studied extensively since its discovery in 1984. Its pro-inflammatory function was manifested in multiple diseases, among them ankylosing spondylitis, IBD, rheumatoid arthritis, psoriasis, systemic lupus erythematosus and juvenile idiopathic arthritis (reviewed in Crit Rev Immunol, 2019. 39(6): 439-479).

AIRs as a new synthetic tool for cellular therapy allow Tregs to sense these environmental signals and translate these into a Treg TCR-like activating program, enabling Treg cells to fulfill their suppressive and tissue protective functions, independent of a specific TCR- or CAR-antigen and independent of endogenous TCR-MHC restriction.

Certain constructs are known in the art that share certain features with the constructs of the present invention. WO2021/051195 for examples discloses constructs that are devoid of a CD3zeta TCR signaling domain. The additional CD3zeta TCR signaling domain however provides several advantages which are beyond the common functionality of a CD3 T cell signaling domain. Due to the presence of the CD3zeta domain, the AIRs of the present invention exert their function directly at the main signal of the signaling cascade. As shown herein, AIRs constructs without the CD3zeta domain are not functional. The constructs of WO2021/051195 are only able to modulate an ancillary signal, and not a TCR-like activation of the cell.

### SUMMARY OF THE INVENTION

The present disclosure provides novel artificial immune receptors capable of activating regulatory T cells into a Treg TCR-like activating program. This program is independent of a specific TCR- or a CAR-antigen and also independent of endogenous TCR-MHC restriction.

Disclosed herein are artificial immune receptors, comprising
a) an extracellular domain of a member of the tumor necrosis factor receptor superfamily,
b) a transmembrane domain,
c) a cytoplasmic costimulatory signaling domain, and
d) a cytoplasmic T cell receptor signaling domain.

The general concept is exemplified utilizing the extracellular domains of four different tumor necrosis factor receptor superfamily members, namely TNFR2, DR3, LTBR and CD40. In certain embodiments the transmembrane domain is derived from the same protein as the extracellular domain.

The artificial immune receptors also comprise a cytoplasmic costimulatory signaling domain and a cytoplasmic T cell receptor signaling domain. Any commonly known costimulatory signaling domain and T cell receptor signaling domain can be used in the context of the AIRs of the present disclosure. Exemplified is the cytoplasmic costimulatory signaling domain of CD28 and the cytoplasmic T cell receptor signaling domain from CD3 zeta.

Particularly preferred artificial immune receptors comprising an extracellular domain and a transmembrane domain of TNFR2, a cytoplasmic costimulatory signaling domain from CD28 and said cytoplasmic T cell receptor signaling domain from CD3 zeta. Yet other particularly preferred artificial immune receptors comprising an extracellular domain and a transmembrane domain of DR3, a cytoplasmic costimulatory signaling domain from CD28 and said cytoplasmic T cell receptor signaling domain from CD3 zeta. Yet other particularly preferred artificial immune receptors comprising an extracellular domain and a transmembrane domain of LTBR, a cytoplasmic costimulatory signaling domain from CD28 and said cytoplasmic T cell receptor signaling domain from CD3 zeta.

Also preferred are artificial immune receptors that comprise the amino acid sequences of SEQ ID No.: 39, SEQ ID No.: 35, and SEQ ID No.: 37.

Also preferred are artificial immune receptors that comprise the amino acid sequences of SEQ ID No.: 41, SEQ ID No.: 35, and SEQ ID No.: 37.

Also preferred are artificial immune receptors that comprise the amino acid sequences of SEQ ID No.: 33, SEQ ID No.: 35, and SEQ ID No.: 37.

Also preferred are artificial immune receptors that comprise the amino acid sequences of SEQ ID No.: 47, SEQ ID No.: 35, and SEQ ID No.: 37. The present disclosure also provides nucleic acids encoding aforementioned artificial immune receptor. The present disclosure also provides vectors comprising said nucleic acids. The present disclosure also provides host cell comprising said nucleic acids, said vectors, as well as host cells expressing the artificial immune receptors of the present disclosure.

The present disclosure also provides the artificial immune receptor for use in medicine. In certain embodiments said use in medicine is the treatment of cancer or inflammation. In other embodiments said use in medicine is the treatment of autoimmunity. In yet other embodiments said use in medicine is the treatment of graft versus host disease or in solid organ transplantation.

### Definitions

The term “cell” as used herein includes a single cell as well as a plurality of cells.

The term “host cell” as used herein refers to a cell comprising a nucleic acid and/or a vector. In the context of the artificial immune receptors of the present disclosure, the term host cell refers to a cell comprising a nucleic acid and/or a vector encoding for an AIR. Such host cell will express the AIR on the cell surface and is suitable to be used as medicine. Preferred host cells of the present invention are eukaryotic host cells, such as immune cells.

The term “T cell” as used herein refers to a type of lymphocyte that plays a central role in cell-mediated immunity. T cells, also referred to as T lymphocytes, can be distinguished from other lymphocytes, such as B cells and natural killer cells, by the presence of a T-cell receptor (TCR) on the cell surface. There are several subsets of T cells with distinct functions, including but not limited to, T helper cells, cytotoxic T cells, memory T cells, regulatory T cells and natural killer T cells. In some embodiments, the T cell is an engineered T cell.

The terms “regulatory T cell” or “Treg” as used herein refer to a subpopulation of T cells which modulate the immune system, maintain tolerance to self-antigens, and abrogate autoimmune diseases. These cells generally suppressor downregulate induction and proliferation of effector T cells. Treg cells are long-lived cells that suppress excessive or uncontrolled immune responses in vivo in a dominant and antigen-specific manner. Genetic mutations in the forkhead box protein 3 (FoxP3), a key transcription factor required for differentiation of Treg cells, lead to severe autoimmunity. Indeed, research in a variety of animal models has demonstrated that Tregs can be used to treat many auto-inflammatory diseases such as type 1 diabetes, inflammatory bowel disease, systemic lupus erythematosus, multiple sclerosis (MS), rheumatoid arthritis, and auto-immune gastritis. Treg cell therapy can also be used in controlling alloimmune responses in the context of GVHD, as well as organ and cell transplantation.

The term “engineered TCR” or “engineered T-cell receptor” means any TCR that has been modified from its naturally-occurring form. An engineered TCR may have modifications to the alpha and/or beta chains, or the gamma and/or delta chains (including replacement of any of the aforementioned chains) that enable the TCR to recognize a specific antigen (for example, a neoantigen). The engineered TCR may have modifications to any CD3 subunit (for example, CD3a, as in the case of TRuC receptors), including the addition of an antigen recognition domain (e.g., an antibody, an scFv, a DARPin). The engineered TCR may have an antigen recognition domain (e.g., an antibody, an scFv, a DARPin) joined to a transmembrane domain of the alpha and/or beta chains, or the gamma and/or delta chains.

The terms “polynucleotide” and/or “nucleic acid sequence” and/or “nucleic acid” as used herein refer to a sequence of nucleoside or nucleotide monomers consisting of bases, sugars and intersugar (backbone) linkages. The term includes DNA and RNA and can be either double stranded or single stranded, and represents the sense or antisense strand. The term also includes modified or substituted sequences comprising non-naturally occurring monomers or portions thereof. The nucleic acid sequences of the present application may be deoxyribonucleic acid sequences (DNA) or ribonucleic acid sequences (RNA) and may include naturally occurring bases including adenine, guanine, cytosine, thymidine and uracil. The sequences may also contain modified bases. Examples of such modified bases include aza and deaza adenine, guanine, cytosine, thymidine and uracil; and xanthine and hypoxanthine. The nucleic acids of the present disclosure may be isolated from biological organisms, formed by laboratory methods of genetic recombination or obtained by chemical synthesis or other known protocols for creating nucleic acids.

The terms “isolated polynucleotide” or “isolated nucleic acid sequence” as used herein refer to a nucleic acid substantially free of cellular material or culture medium when produced by recombinant DNA techniques, or chemical precursors, or other chemicals when chemically synthesized.

The terms “recombinant nucleic acid” or “engineered nucleic acid” as used herein refer to a nucleic acid or polynucleotide that is not found in a biological organism. For example, recombinant nucleic acids may be formed by laboratory methods of genetic recombination (such as molecular cloning) to create sequences that would not otherwise be found in nature. Recombinant nucleic acids may also be created by chemical synthesis or other known protocols for creating nucleic acids. Unless otherwise indicated, the definitions and embodiments described in this and other sections are intended to be applicable to all embodiments and aspects of the present application herein described for which they are suitable as would be understood by a person skilled in the art.

The term “polypeptide” or “protein” as used herein describes a chain of amino acids. A polypeptide or protein of this disclosure can be a peptide, which usually describes a chain of amino acids of from two to about 30 amino acids. The term protein as used herein also describes a chain of amino acids having more than 30 amino acids and can be a fragment or domain of a protein or a full length protein. Furthermore, as used herein, the term protein can refer to a linear chain of amino acids or it can refer to a chain of amino acids that has been processed and folded into afunctional protein. It is understood, however, that 30 is an arbitrary number with regard to distinguishing peptides and proteins and the terms can be used interchangeably for a chain of amino acids. The proteins of the present disclosure can be obtained by isolation and purification of the proteins from cells where they are produced naturally, by enzymatic (e.g., proteolytic) cleavage, and/or recombinantly by expression of nucleic acid encoding the proteins or fragments of this disclosure. The proteins and/or fragments of this disclosure can also be obtained by chemical synthesis or other known protocols for producing proteins and fragments.

The term “isolated polypeptide” refers to a polypeptide substantially free of cellular material or culture medium when produced by recombinant DNA techniques, or chemical precursors or other chemicals when chemically synthesized.

The term “vector” as used herein refers to a polynucleotide that can be used to deliver a nucleic acid to the inside of a cell. In one embodiment, a vector is an expression vector comprising expression control sequences (for example, a promoter) operatively linked to a nucleic acid to be expressed in a cell. Vectors known in the art include, but are not limited to, plasmids, phages, cosmids and viruses.

The terms “recipient”, “individual”, “subject”, “host”, and “patient”, are used interchangeably herein and refer to any mammalian subject for whom diagnosis, treatment, or therapy is desired, particularly humans.

As used herein, the terms “treatment,” “treating,” and the like, in some embodiments, refer to administering an agent, or carrying out a procedure, for the purposes of obtaining an effect. The effect may be prophylactic in terms of completely or partially preventing a disease or symptom thereof and/or may be therapeutic in terms of affecting a partial or complete cure for a disease and/or symptoms of the disease. The terms include treatment of a disease or disorder (e.g. inflammation) in a mammal, particularly in a human, and includes: (a) preventing the disease or a symptom of a disease from occurring in a subject which may be predisposed to the disease but has not yet been diagnosed as having it (e.g, including diseases that may be associated with or caused by a primary disease; (b) inhibiting the disease, i.e., arresting its development; and (c) relieving the disease, i.e., causing regression of the disease. The treatment or amelioration of symptoms is based on one or more objective or subjective parameters; including the results of an examination by a physician. Accordingly, the term “treating” includes the administration of the compounds or agents of the present invention to prevent, delay, alleviate, arrest or inhibit development of the symptoms or conditions associated with diseases (e.g. inflammation).

The term “therapeutic effect” refers to the reduction, elimination, or prevention of the disease, symptoms of the disease, or side effects of the disease in the subject.

The terms “tumor necrosis factor receptor superfamily”, “TNFR superfamily” or “TNFRSF” as used herein refers the protein superfamily of cytokine receptors characterized by the ability to bind ligands of tumor necrosis factor super family members (TNFSF) via an extracellular domain. There are numerous members of the TNFR Superfamily, including TNFR1, TNFR2, Fas, DR4, DR5, DR3, DR6, EDAR, XEDAR, TROY LTBR or NGFR. Other members includes lymphotoxin beta receptor (CD18), Ox40 (CD134), CD40 (TNFRSF5), decoy receptor 3 (TR6, M68), CD27 (S152, Tp55), CD30 (Ki-1, TNR8), 4-1BB (CD137), decoy receptor 1 (TRAILR3), decoy receptor 2 (TRAILR4), RANK (CD265), osteoprotegerin (OCIF, TR1), TWEAK receptor (Fn14, CD266), TACI (IGAD2, CD267), BAFF receptor (CD268), BAFF, APRIL, herpesvirus entry mediator (HVEM, CD270), B cell maturation antigen (TNFRSF13A, CD269), and glucocorticoid-induced TNFR-related protein (AITR, CD357).

Tumor necrosis factor receptor 1 (NCBI Entrez Gene: 7132; also known as TNFR1, tumor necrosis factor receptor superfamily member 1A, TNFRSF1A or CD 120a) is a membrane-bound receptor that binds tumor necrosis factor-alpha (TNFa). TNFR1 activates the transcription factor NF-kB, mediates apoptosis, and functions as a regulator of inflammation.

Tumor necrosis factor receptor 2 (NCBI Entrez Gene: 7133; also known as TNFR2, tumor necrosis factor receptor superfamily member 1B, TNFRSF1B or CD120b9, is a membrane-bound receptor that binds tumor necrosis factor-alpha (TNFa).

The Fas receptor (NCBI Entrez Gene: 355, also known as Fas, FasR, apoptosis antigen 1, APO-1, APT, CD95, tumor necrosis factor receptor superfamily member 6 or TNFRSF6), is a protein that in humans is encoded by the FAS gene. Multiple splice variants of Fas have been identified, which are translated into seven isoforms of the protein. Apoptosis inducing Fas receptor is referred to as isoform 1 and is a type 1 transmembrane protein. Many of the other isoforms are rare haplotypes that are usually associated with a state of disease. Any suitable isoform of Fas is contemplated for use with the embodiments disclosed herein.

Death receptor 4 (NCBI Entrez Gene: 8797, also known as death domain 4, DR4, TRAIL receptor 1, TRAILR1, tumor necrosis factor receptor superfamily member 10A or TNFRSF 10A), is a cell surface receptor of the TNF-receptor superfamily that binds TRAIL and mediates apoptosis.

Death domain 5 (NCBI Entrez Gene: 8795, also known as death receptor 5, DR5, TRAIL receptor 2, TRAILR2, tumor necrosis factor receptor superfamily member 10B or TNFRSF10B) is a cell surface receptor of the TNF-receptor superfamily that binds TRAIL and mediates apoptosis.

Death domain 3 (NCBI Entrez Gene: 8718, also known as death receptor 3, DR3, tumor necrosis factor receptor superfamily member 25 or TNFRSF25) is a cell surface receptor of the tumor necrosis factor receptor superfamily which mediates apoptotic signaling and differentiation. Its only known TNFSF ligand is TNF-like protein 1A (TL1A).

Death domain 6 (NCBI Entrez Gene: 27242, also known as death receptor 6, DR6, tumor necrosis factor receptor superfamily member 21 or TNFRSF21), is a cell surface receptor of the tumor necrosis factor receptor superfamily which activates the JNK and NF-κB pathways.

Ectodysplasin receptor A (NCBI Entrez Gene: 10913, also known as ectodermal dysplasia receptor, EDA-A1 or EDAR) is a member of the TNF-receptor superfamily. It plays a key role in the process of ectodermal differentiation.

Ectodysplasin A2 receptor (NCBI Entrez Gene: 60401, also known as XEDAR, EDAR2, EDA-A2 or Tumor necrosis factor receptor superfamily member 27) is a protein that in humans is encoded by the EDA2R gene. EDA-A1 and EDA-A2 are two isoforms of ectodysplasin that are encoded by the anhidrotic ectodermal dysplasia (EDA) gene.

TROY (NCBI Entrez Gene: 55504, also known as Tumor necrosis factor receptor superfamily member 19 or TNFRSF19) is a member of the TNF-receptor superfamily. This receptor is highly expressed during embryonic development. It has been shown to interact with TNF receptor associated factor (TRAF) family members, and to activate c-Jun N-terminal kinases (JNK) signaling pathway when overexpressed in cells. This receptor is capable of inducing apoptosis by a caspase-independent mechanism, and it is thought to play an essential role in embryonic development.

NGFR (NCBI Entrez Gene: 4804, also known as nerve growth factor receptor, TNFR superfamily member 16, TNFRSF16, LNGFR or p75 neurotrophin receptor) is a member of the tumor necrosis factor receptor (TNF receptor) superfamily. It is one of the two receptor types for the neurotrophins, a family of protein growth factors that stimulate neuronal cells to survive and differentiate.

LTBR (NCBI Entrez Gene: 4055, also known as lymphotoxin beta receptor, TNFRSF3, TNFCR, Tumor Necrosis Factor C Receptor of TNFR3) plays a role in signaling during the development of lymphoid and other organs, lipid metabolism, immune response, and programmed cell death.

CD40 (NCBI Entrez Gene: 958, also known as Bp50, Tumor Necrosis Factor Receptor Superfamily Member 5 or TNFRSF5) is a costimulatory protein found on antigen-presenting cells and is required for their activation. CD40 binds CD154 (CD40L) on TH cells, thereby activating antigen presenting cells and inducing a variety of downstream effects.

CD30 (NCBI Entrez Gene: 943, also known as D1S166E, Tumor Necrosis Factor Receptor Superfamily Member 8, TNFRSF8, CD30L Receptor or KI-1) is expressed by activated, but not by resting, T and B cells and interacts with TRAF2 and TRAF5.

BAFF (NCBI Entrez Gene: 10673, also known as TALL-1, Tumor Necrosis Factor Receptor Superfamily Member 13B, TNFRSF13B, THANK or CD257) is a ligand for various receptors. BAFF is expressed in B cell lineage cells, and acts as a B cell activator. It has been also shown to play an important role in the proliferation and differentiation of B cells.

APRIL (NCBI Entrez Gene: 8741, also known as CD256, Tumor Necrosis Factor Receptor Superfamily Member 13, TNFRSF13, TALL-2, TRDL-2 or ZNTF2) is recognized by the cell surface receptor TACI, and, together with its receptor, plays an important for B cell development.

The terms “costimulatory molecule” or “costimulatory receptor” as used herein refers to a cognate binding partner on a T cell that specifically binds with a costimulatory ligand, thereby mediating a costimulatory response in the T cell, such as, but not limited to, activation or proliferation. A co-stimulatory receptor may be expressed on cells other than T cells, such as NK cells or macrophages. A costimulatory molecule can be represented in the following protein families: TNF receptor proteins, Immunoglobulin like proteins, cytokine receptors, integrins, signaling lymphocytic activation molecules (SLAM proteins), Toll-like receptors and NK cell receptors. Costimulatory molecules include, but are not limited to 4-IBB (CD137), BAFFR, OX40, CD27, CD28, CD40, ICOS, 2B4, GITR, HVEM, OX40, RELT, TACI, TROY, TWEAK, KIR receptors, TLR1 to TLR9 receptors, IL-2, IL-7 and IL-15 receptors.

The terms “costimulatory signaling domain” or “costimulatory domain” as used herein refer to the domain of a costimulatory molecule or costimulatory receptor responsible for mediating a costimulatory response by the T cell. The intracellular signaling domain can comprise the entire intracellular portion, or the entire native intracellular signaling domain, of the molecule from which it is derived, or a functional fragment or derivative thereof.

The terms “T cell receptor signaling domain” or “TCR signaling domain” as used herein refers to cytoplasmic signaling sequence that acts in a stimulatory manner to induce immune effector functions. In some embodiments, the TCR signaling domain contains a signaling motif known as Immunoreceptor Tyrosine-based Activation Motif, or ITAM. In some embodiments, the primary intracellular signaling domain comprises afunctional signaling domain of a protein selected from the group consisting of CD3 zeta, CD3 gamma, CD3 delta, CD3 epsilon, common FcR gamma (FCER1G), FcR beta (Fc Epsilon Rib), CD79a, CD79b, Fcgamma RIIa, DAP10, and DAP12. A preferred TCR signaling domain is a TCR signaling domain selected from CD3 zeta, CD3 gamma, CD3 delta and CD3 epsilon. A particularly preferred TCR signaling domain CD3 zeta, CD3 gamma, CD3 delta and CD3 epsilon.

The terms “artificial immune receptor” or “AIR” refer to the novel molecules of the present disclosure. AIRs comprise an extracellular domain of a member of the tumor necrosis factor receptor superfamily, a transmembrane domain, a cytoplasmic costimulatory signaling domain, and a cytoplamsic T cell receptor signaling domain.

The terms “is”, “are”, “is derived from” and “are derived from” in the context of a polypeptide or domain of a polypeptide refers to the amino acid sequence of said polypeptide or domain of a polypeptide and indicates that the amino acid sequence is either identical to the native version of said polypeptide or domain of a polypeptide, or a variant of said polypeptide or domain of a polypeptide which is functionally indistinguishable form from the native version of said polypeptide or domain of a polypeptide.

The term “P2A” as used herein refers to a peptide sequence which is capable of self cleavage. P2A belongs to the family of 2A peptides, which all are capable of self cleavage. “A peptides share a common core sequence motif. Any 2A peptide may be used instead of P2A. The amino acid sequence of P2A is shown in SEQ ID No. 13. Other commonly used 2A peptides are T2A, E2A and F2A.

The term “CD90.1” as used herein refers to an allelic isoform of mouse CD90. The amino acid sequence of mouse CD90.1 is shown in SEQ ID No. 15. CD90.1 is commonly used genetic marker.

The terms “GVHD”, “GvHD” and “Graft versus Host Disease” refers to a disease commonly associated with bone marrow transplants and stem cell transplants, characzerized by inflammation. White blood cells of the donor's immune system remain within the donated tissue or the donated cells (the graft) and are recognized by the recipient (the host) as non-self.

### FIGURE LEGENDS

FIG. 1 is a schematic representation of the AIR design. The construct on the top (A) shows an AIR according to the present disclosure. It consist of an extracellular binding domain of a Tumor-Necrosis-Factor (TNF) superfamily member, a transmembrane domain, and two intracellular domains-one co-stimulatory domain and one T cell receptor signaling domain. The AIR fusion proteins are linked via a P2A peptide cleavage site to modified CD90.1, which serves as reporter gene for transduction efficacy. P2A and CD90.1 are not part of the AIRs. These constructs are cloned into a Murine Stem Cell Leukemia Virus (MSCV), which serves as vector and mediates stable integration of genes of interest into T cell DNA. Constructs designated as B, C, D and G are exemplary AIRs according to the present disclosure. Constructs E and F are control constructs. Construct E lacks an extracellular binding domain, construct F contains an extracellular anti-CD19 single chain antibody (scFv) instead of a binding domain of a Tumor-Necrosis-Factor (TNF) superfamily member.

FIG. 2 is depicts the mode of action of the AIRs according to the present disclosure. Binding of membrane bound TNF family members via AIRs leads to translation of the cytokine signal into a T cell receptor (TCR)-like signal. This initiates T cell activation independent of its endogenous TCR-MHC restriction in an environment/inflammation dependent manner.

FIG. 3 demonstrates efficient transduction and expression of the AIRs according to the present disclosure. Murine regulatory T cells (Treg) were isolated from spleen and lymph nodes of C57/BL6 mice, sorted (naïve CD4+CD25+CD62L+ Treg or Foxp3+ reporter (hCD2+) positive) and expanded via anti-CD3 and anti-CD28 antibody coupled beads plus IL-2 (2000 U/ml) stimulation for 6 days. Purity of Treg cultures was determined with flow cytometry. Treg cultures contained about 90-99% Foxp3+ T cells. On day two after isolation Treg were transduced with AIR constructs or an anti-CD19 Chimeric Antigen Receptor (α-CD19 CAR) as control. Three days later transduction efficacy via CD90.1 staining and surface expression of AIRs was checked with flow cytometry.

FIG. 4 demonstrates the surface expression of the AIRs according to the present disclosure. Panels A, B and C show the results with AIRs carrying the extracellular domain of LTBR, DR3 and TNFR2, respectively. In each panel the AIRs are compared to untransduced cells and to cells carrying an extracellular anti-CD19 single chain antibody (scFv).

FIG. 5 depicts the scientific rational for the experiment of Example 6. Nr4a1 is a well-known target gene of T-cell receptor signaling. It was investigated if the AIRs of the present disclosure can induce Nr4a1 upregulation in a similar way as TCR signaling. Treg from Nr4a1-eGFP reporter mice were used. As a control, Treg were stimulated with anti-CD3/CD28 antibody-coupled beads.

FIG. 6 AIRs expressing Treg were stimulated in over night co-culture with HEK cells expressing murine Light (mLight), mTL1A or mTNF. As positive control Treg were stimulated with anti-CD3/CD28 antibody-coupled beads to mimic TCR signaling. Nr4a1-eGFP expression of AIR positive (CD90.1+) Treg is shown in the left, Nr4a1-eGFP expression of AIR negative (CD90.1−) Treg in the right panel.

FIG. 7 demonstrates the expression of Nr4a1 upon administration of an AIR according to the present disclosure (exemplified with an AIR carrying carrying the extracellular domain of LTBR. Tregs expressing an LTBR AIR according to the present disclosure or an irrelevant CAR carrying extracellularly an anti-CD19 single chain antibody (scFv) were co-cultured with murine EL4 cells, which are known to express Light, in absence or presence of a mLight blocking LTBR-lg fusion protein (50 ug/ml) and afterwards stained for intracellular Nr4a1 expression.

FIG. 8 shows the pre-gating strategy for CD90.1+ cells of LTBR AIR's according to the present disclosure and an irrelevant CAR carrying extracellularly an anti-CD19 single chain antibody (scFv). Tregs expressing the constructs were rested for 24 h and then co-cultured for 18 h with HEK cells or HEK cells expressing mLight. Shown is the pregating for CD90.1+ cells.

FIG. 9 shows the sorting of Tregs that were pregated as shown in FIG. 8. Cells were sorted on CD4+CD25+CD90.1+ (left and middle) or CD4+CD25+CD90.1+Nr4a1+LAP+ (right).

FIG. 10 depicts an RNA expression analysis differentially expressed transcripts after stimulation with LTBR AIR's according to the present disclosure (comparison of LTBR AIR Treg's stimulated with HEK-mLight vs. α-CD19 CAR Tregs stimulated with HEK-mLight). Cells were sorted as outlined in FIGS. 8 and 9 and in Example 7. Afterwards RNA was isolated and RNA sequencing was performed. Shown on the left is a summary of up and down regulated genes comparing α-CD19 CAR Treg stimulated with HEK-mLight vs. LTBR AIR Treg stimulated with HEK cells as control (endogenous mLight signaling) and LTBR AIR Treg stimulated with HEK-mLight vs. α-CD19CAR Treg stimulated with HEK-mLight (specific LTBR AIR signaling). Shown on the right is a Volcano plot showing differentially expressed transcripts.

FIG. 11 is analogous to FIG. 10 with DR3 AIR's according to the present disclosure. DR3 AIR Tregs were stimulated with HEK cells (endogenous mTL1A signaling). DR3AIR Treg were stimulated with HEK-mTL1A vs. α-CD19 CAR Treg stimulated with HEK-mTL1A (specific DR3 AIR signaling). Differentially expressed gene are shown on the left, a Volcano plot is shown on the right.

FIG. 12 is also analogous to FIG. 10 comparing α-CD19 CAR Treg stimulated with anti-CD3/CD28 beads vs. unstimulated Treg. Differentially expressed gene are shown on the left, a Volcano plot is shown on the right.

FIG. 13 shows RNA expression data from LTBR AIR or control CAR expressing Treg for Tnfrsf9, Tigit, Tgfb1, Cd69, Ccr8 and Nr4a1 after 18 h co-culture with HEK+/−mLight (Deseq2, n=3).

FIG. 14 shows a representative flow cytometry analysis from LTBR AIR or control CAR expressing Treg after 18 h co-culture with HEK+/−mLight. Protein expression of CD137 and Tigit is shown in the upper, CD69 and LAP (membrane-bound Tgfβ1) in the lower panel. Data is representative for three independent experiments.

FIG. 15 shows RNA expression data from DR3 AIR or control CAR expressing Treg for Tnfrsf9, Tigit, Tgfb1 after 18 h co-culture with HEK+/−mLight (Deseq2, n=3).

FIG. 16 shows a representative flow cytometry analysis from DR3AIR or control CAR expressing Treg after 18 h co-culture with HEK+/−mTL1A. Protein expression of CD137 and Tigit is shown in the upper, CD69 and LAP (Tgfβ1) in the lower panel. Data is representative for three independent experiments.

FIG. 17 shows RNA expression data (upper left, Deseq2, n=3) and protein expression (right) from LTBR AIR or control CAR expressing Treg for Irf8 after 18 h co-culture with HEK+/−mLight. RNA expression data (lower left, Deseq2, n=3) and protein expression (right) from DR3 AIR or control CAR expressing Treg for Irf8 after 18 h co-culture with HEK+/−mTL1A.

FIG. 18 demonstrates that the AIRs of the present disclosure mediate Treg activation and proliferation. LTBR AIR or control CAR expressing Treg were rested for 24 h and labeled with CFDA-SE proliferation dye. Engineered and labeled Treg were co-cultured with HEK+/−mLight for 72 h in presence of IL2 and afterwards analyzed with flow cytometry for proliferation. Representative dot plots are shown left. Summarized data from three experiments performed in three technical replicates are shown right (n=3).

FIG. 19 shown a schematic overview of the (major MHC mismatch) Graft versus Host Diseases (GvHD) model. Briefly, FACS sorted Treg from Foxp3-hCD2 reporter mice were expanded and transduced with LTBR AIR or a truncated construct (lacking an extracellular binding domain, shown in FIG. 1 a) as control. 2.5×10⁵ engineered Treg were transplanted together with 2.5×10⁶ bone marrow cells and 2.5×10⁵ spleen cells from C57/BL6 mice into lethally irradiated Balb/c mice. As transplant control, one group received only bone marrow cells (BM) and as disease developing control, on group received bone marrow cell plus spleen cells. After transplantation, animals were monitored for 47 days.

FIG. 20 shows the result of the quality control of the engineered Tregs. Tregs were stained for CD4, hCD2 (reporter for Foxp3) as well as CD90.1 and LTBR before transfer into mice.

FIG. 21 shows a Kaplan-Meier curve showing survival of transplanted Balb/c mice treated with a LTBR AIR and respective control constructs. Graph contains data set from two independent experiments (log rank test, n=11-12).

FIG. 22 shows the mean GvHD score per group, animals reaching a score of 40 had to be euthanized (dead animals kept the highest score over following time points for statistical analysis, two-way ANOVA).

FIG. 23 shows on the left a representative FACS plot of a spleen of a surviving LTBR AIR Treg receiving animal. Shown on the right is the percentage of Foxp3+ Tregs in spleen of surviving mice at day 47.

FIG. 24 shows the analysis of KIrg1+ Treg in spleens. Left: representative FACS plot of discriminated CD45.1+ (BM derived) and CD45.2+ (transferred AIR Treg) Treg cells. Expression of KIrg1 (marker for tissue specific phenotype) and hCD2 (Foxp3 reporter) is shown. Middle: KIrg1+ expression of engineered CD45.2+ or BM derived CD45.1+ Treg in surviving mice. Right: frequency of KIrg1+hCD2+ Treg in mice receiving LTBR AIR Treg (Mann-Whitney-U, survivors n=8 vs. dead n=4).

FIG. 25 shows a representative FACS analysis of a digested colon of a surviving LTBR AIR Treg receiving animal. Shown is discrimination between CD45.1 and CD45.2 cells within the CD4+ population on the left. CD45.2 Treg are further analyzed for KIrg1 and hCD2 expression on the right.

FIG. 26 shows on the left the percentage of hCD2+ (indicating Foxp3+) cells among transferred engineered CD45.2+ cells demonstrating phenotypic stability over 47 day in vivo. Shown on the right is the cell count of transferred CD45.2 Treg in colon at day 47.

FIG. 27 shows that human LTBR AIRs are efficiently transduced and express the AIR on the cell surface. Human T cells from healthy blood donors were sorted on the typical Treg protein markers (TCRb+CD4+CD25+CD127-CD45RA+) and expanded with anti-CD3/CD28 stimulation (TransACT, Miltenyi Biotec, Bergisch Gladbach, Germany) and IL-2 (500 U/ml) for 7 days. On day 2 after isolation Treg were transduced with the human LTBR AIR construct (using an amphotropic version of the retroviral expression system MSCV). 5 days later cultured Treg were stained for intracellular Foxp3+ to check for Treg purity and for CD90.1 as well as hLTBR to analyze transduction efficacy and surface expression of the AIR protein.

FIG. 28 shows that human LTBR AIRs have the expected signaling capacity. Tregs expressing hLTBR AIR or irrelevant CAR (an anti-CEA, Carcinoembrionic Antigen) were co-cultured for 18 h with parental HEK cells or HEK cells expressing human Light protein on the surface. Expression of Ccr8, Glycoprotein A repetitions predominant (GARP), CD137 (4-1BB) and Tigit is shown in CD90.1+Foxp3+ Tregs.

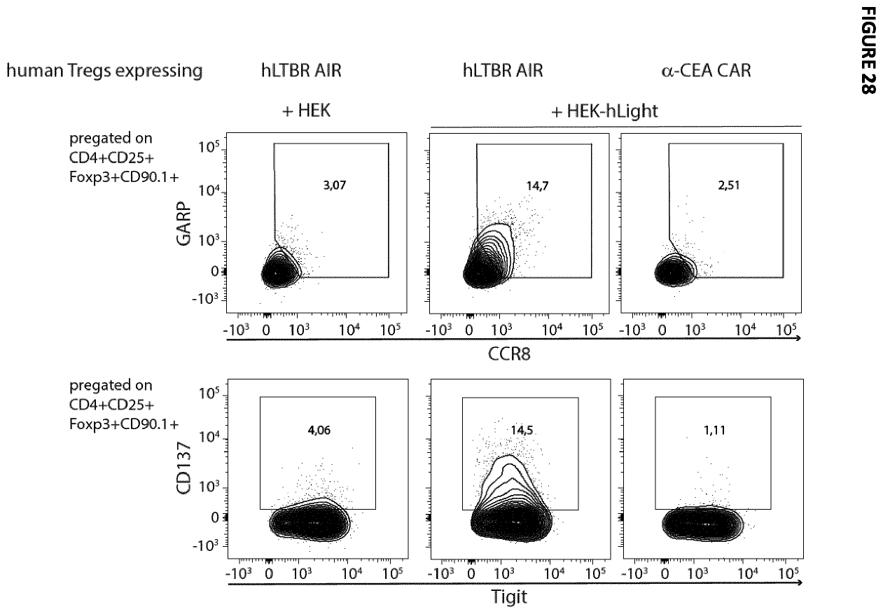

FIG. 29 shows on the top a LTBR “AIR” lacking the CD3zeta-chain. On the bottom, flow cytometry data are shown which confirm surface expression of the construct in Tregs.

FIG. 30 shows a representative flow cytometry analysis of Tregs expressing a full length LTBR AIR with a LTBR “AIR” lacking the CD3zeta-chain after 18 h co-culture with HEK+/−mLight. Protein expression of CD137 and Tigit is shown in the upper, CD69 and LAP (membrane-bound Tgf [symbol] 1) in the lower panel.

FIG. 31 shows the expression of TIGIT and CD137. CD40 AIR-expressing or a construct containing an anti-CD19 CAR expressing Treg cells were cocultured for 18 h with HEK cells or HEK cells expressing CD40L (CD154). Expression of TIGIT and CD137 was measured by flow cytometry.

FIG. 32 summarizes three independent experiments as shown in FIG. 31. Differences are statistically significant as calculated via a one-way Anova test.

FIG. 33 shows the expression of LAP (TGFb1) and CD69. CD40 AIR-expressing or a construct containing an anti-CD19 CAR expressing Treg cells were cocultured for 18 h with HEK cells or HEK cells expressing CD40L (CD154). Expression of LAP (TGFb1) and CD69 was measured by flow cytometry.

FIG. 34 summarizes three independent experiments as shown in FIG. 33. Differences are statistically significant as calculated via a one-way Anova test.

FIG. 35 shows that Treg cells from Nr4a1-eGFP reporter mice expressing a CD40 AIR trigger the expression of Nr4a1, an early response gene of TCR signaling.

FIG. 36 shows a schematic representation of additional AIRs generated and tested in the present disclosure. FIG. 36A shown a CD40 AIR with an ICOS costimulatory domain, FIG. 36B a CD40 AIR with a 4-1BB costimulatory domain.

FIG. 37 shows that CD40 AIRs with an ICOS or a 41BB costimulatory domain are efficiently expressed on the surface of regulatory T cells.

FIG. 38 shows that all CD40 AIRs tested, i.e. AIRs with a CD28, an ICOS and a 41BB costimulatory domain, have the capacity to induce Treg cell activation.

FIG. 39 shows the quality control of Teg cells prior to transplantation. Both construct, the CD40 AIR and the control construct, showed a high transduction efficiency, but only the cells transduced with the CD40AIR exhibited binding to CD40 through its CD40 receptor extracellular domain.

FIG. 40 shows a Kaplan-Meier curve showing survival of transplanted Balb/c mice treated with a CD40 AIR and respective control constructs. Graph contains data set from two independent experiments (log rank test, n=10-12).

FIG. 41 demonstrates the importance of the CD3 for the constructs of the present invention. Only the full length LTBR AIR containing the CD3zeta domain, but not the other two constructs tested, is capable to induce Nr4a1.eGFP upregulation in response to the corresponding ligand, mLIGHT.

### EMBODIMENTS OF THE INVENTION

### Artificial Immune Receptors

The artificial immune receptors disclosed herein are novel and superior tools that enable regulatory T cells to fulfill their suppressive and tissue protective functions, independent of a specific TCR- or CAR-antigen and independent of endogenous TCR-pMHC (peptide loaded MHC) restriction.

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain.

In certain embodiments the present disclosure relates to an artificial immune receptor, consisting of
a) an extracellular domain of a member of the tumor necrosis factor receptor superfamily;
b) a transmembrane domain,
c) a cytoplasmic costimulatory signaling domain, and
d) a cytoplasmic T cell receptor signaling domain.

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain,
   wherein said member of the tumor necrosis factor receptor superfamily is selected from TNFR1, TNFR2, Fas, DR4, DR5, DR3, DR6, EDAR, XEDAR, TROY, LTBR, NGFR, CD18, CD134, CD40, CD27, CD30, CD137, TRAILR3, TRAILR4, CD265, osteoprotegerin, CD266, TACI, BAFF, BAFF receptor, APRIL, CD270, CD269 and CD357.

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain,
   wherein said member of the tumor necrosis factor receptor superfamily is selected from TNFR2, DR3, LTBR and CD40.

In certain embodiments the present disclosure relates to an artificial immune receptor, consisting of
a) an extracellular domain of a member of the tumor necrosis factor receptor superfamily;
b) a transmembrane domain,
c) a cytoplasmic costimulatory signaling domain, and
d) a cytoplasmic T cell receptor signaling domain,
   wherein said member of the tumor necrosis factor receptor superfamily is selected from TNFR1, TNFR2, Fas, DR4, DR5, DR3, DR6, EDAR, XEDAR, TROY, LTBR, NGFR, CD18, CD134, CD40, CD27, CD30, CD137, TRAILR3, TRAILR4, CD265, osteoprotegerin, CD266, TACI, BAFF, BAFF receptor, APRIL, CD270, CD269 and CD357.

In certain embodiments the present disclosure relates to an artificial immune receptor, consisting of
a) an extracellular domain of a member of the tumor necrosis factor receptor superfamily;
b) a transmembrane domain,
c) a cytoplasmic costimulatory signaling domain, and
d) a cytoplasmic T cell receptor signaling domain,
   wherein said member of the tumor necrosis factor receptor superfamily is selected from TNFR2, DR3, LTBR and CD40.

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain,
   wherein said transmembrane domain is from the same protein as the extracellular domain.

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain,
   wherein said transmembrane domain and said extracellular domain are derived from the same protein.

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain,
   wherein said member of the tumor necrosis factor receptor superfamily and said transmembrane domain are derived from TNFR2.

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain,
   wherein artificial immune receptor comprises SEQ ID No.: 39.

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain,
   wherein artificial immune receptor comprises SEQ ID No.: 38.

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain,
   wherein artificial immune receptor consists of SEQ ID No.: 38.

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain,
   wherein said member of the tumor necrosis factor receptor superfamily and said transmembrane domain are derived from DR3.

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain,
   wherein artificial immune receptor comprises SEQ ID No.: 41.

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain,
   wherein artificial immune receptor comprises SEQ ID No.: 40.

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain,
   wherein artificial immune receptor consists of SEQ ID No.: 40.

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain,
   wherein said member of the tumor necrosis factor receptor superfamily and said transmembrane domain are derived from LTBR.

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain,
   wherein artificial immune receptor comprises SEQ ID No.: 33.

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain,
   wherein artificial immune receptor comprises SEQ ID No.: 31.

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain,
   wherein artificial immune receptor consists of SEQ ID No.: 31.

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain,
   wherein said member of the tumor necrosis factor receptor superfamily and said transmembrane domain are derived from CD40.

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain,
   wherein artificial immune receptor comprises SEQ ID No.: 46.

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain,
   wherein artificial immune receptor comprises SEQ ID No.: 47.

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain,
   wherein artificial immune receptor consists of SEQ ID No.: 47.

### Costimulatory Signaling Domain

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain.
   Wherein said cytoplasmic costimulatory signaling domain is selected from 4-IBB (CD137), BAFFR, OX40, CD27, CD28, ICOS, CD40, 2B4, GITR, HVEM, OX40, RELT, TACI, TROY, and TWEAK.

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain.
   Wherein said cytoplasmic costimulatory signaling domain is CD28.

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain.
   Wherein said cytoplasmic costimulatory signaling domain is derived from CD28.

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain.
   Wherein said cytoplasmic costimulatory signaling domain comprises SEQ ID No.: 35.

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain.
   Wherein said cytoplasmic costimulatory signaling domain consists of SEQ ID No.: 35.

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain,
   wherein said transmembrane domain is from the same protein as the extracellular domain, and
   wherein said cytoplasmic costimulatory signaling domain is selected from CD137, BAFFR, OX40, CD27, CD28, CD40, ICOS, 2B4, GITR, HVEM, OX40, RELT, TACI, TROY, and TWEAK.

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain,
   wherein said transmembrane domain is from the same protein as the extracellular domain, and
   wherein said cytoplasmic costimulatory signaling domain is CD28.

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain,
   wherein said transmembrane domain is from the same protein as the extracellular domain, and
   wherein said cytoplasmic costimulatory signaling domain comprises SEQ ID No.: 35.

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain,
   wherein said transmembrane domain is from the same protein as the extracellular domain, and
   wherein said cytoplasmic costimulatory signaling domain consists of SEQ ID No.: 35.

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain,
   wherein said transmembrane domain and said extracellular domain are from TNFR2, and
   wherein said cytoplasmic costimulatory signaling domain is CD28.

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain,
   wherein said transmembrane domain and said extracellular domain are from DR3, and
   wherein said cytoplasmic costimulatory signaling domain is CD28.

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain,
   wherein said transmembrane domain and said extracellular domain are from LTBR, and
   wherein said cytoplasmic costimulatory signaling domain is CD28.

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain,
   wherein said transmembrane domain and said extracellular domain are from CD40, and
   wherein said cytoplasmic costimulatory signaling domain is CD28.

### T Cell Receptor Signaling Domain

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain, and
   wherein said cytoplasmic T cell receptor signaling domain is selected from CD3 zeta, CD3 gamma and CD3 epsilon.

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain, and
   wherein said cytoplasmic T cell receptor signaling domain is CD3 zeta.

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain, and
   wherein said cytoplasmic T cell receptor signaling domain is derived from CD3 zeta.

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain, and
   wherein said cytoplasmic T cell receptor signaling domain comprises SEQ ID No.: 37.

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain, and
   wherein said cytoplasmic T cell receptor signaling domain consists of SEQ ID No.: 37.

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain, and
   wherein said transmembrane domain is from the same protein as the extracellular domain, and
   wherein said cytoplasmic T cell receptor signaling domain is CD3 zeta.

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain, and
   wherein said member of the tumor necrosis factor receptor superfamily and said transmembrane domain are derived from TNFR2, and
   wherein said cytoplasmic T cell receptor signaling domain is CD3 zeta.

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain, and
   wherein said member of the tumor necrosis factor receptor superfamily and said transmembrane domain are derived from DR3, and
   wherein said cytoplasmic T cell receptor signaling domain is CD3 zeta.

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain, and
   wherein said member of the tumor necrosis factor receptor superfamily and said transmembrane domain are derived from LTBR, and
   wherein said cytoplasmic T cell receptor signaling domain is CD3 zeta.

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain, and
   wherein said member of the tumor necrosis factor receptor superfamily and said transmembrane domain are derived from CD40, and
   wherein said cytoplasmic T cell receptor signaling domain is CD3 zeta.

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain, and
   wherein said transmembrane domain is from the same protein as the extracellular domain,
   wherein said cytoplasmic costimulatory signaling domain is CD28, and
   wherein said cytoplasmic T cell receptor signaling domain is CD3 zeta.

### Preferred AIRs

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain, and
   wherein said member of the tumor necrosis factor receptor superfamily and said transmembrane domain are derived from TNFR2,
   wherein said cytoplasmic costimulatory signaling domain is CD28, and
   wherein said cytoplasmic T cell receptor signaling domain is CD3 zeta.

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain, and
   wherein said member of the tumor necrosis factor receptor superfamily and said transmembrane domain are derived from DR3,
   wherein said cytoplasmic costimulatory signaling domain is CD28, and
   wherein said cytoplasmic T cell receptor signaling domain is CD3 zeta.

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain, and
   wherein said member of the tumor necrosis factor receptor superfamily and said transmembrane domain are derived from LTBR,
   wherein said cytoplasmic costimulatory signaling domain is CD28, and
   wherein said cytoplasmic T cell receptor signaling domain is CD3 zeta.

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain, and
   wherein said member of the tumor necrosis factor receptor superfamily and said transmembrane domain are derived from CD40,
   wherein said cytoplasmic costimulatory signaling domain is CD28, and
   wherein said cytoplasmic T cell receptor signaling domain is CD3 zeta.

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain, and
   wherein said member of the tumor necrosis factor receptor superfamily and said transmembrane domain are from TNFR2 and comprise SEQ ID No.: 39,
   wherein said cytoplasmic costimulatory signaling domain is CD28 and comprise SEQ ID No.: 35, and
   wherein said cytoplasmic T cell receptor signaling domain is CD3 zeta and comprise SEQ ID No.: 37.

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain, and
   wherein said member of the tumor necrosis factor receptor superfamily and said transmembrane domain are from TNFR2 and consist of SEQ ID No.: 39,
   wherein said cytoplasmic costimulatory signaling domain is CD28 and consists of SEQ ID No.: 35, and
   wherein said cytoplasmic T cell receptor signaling domain is CD3 zeta and consists of SEQ ID No.: 37.

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain, and
   wherein said artificial immune receptor comprises SEQ ID No.: 38.

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain, and
   wherein said artificial immune receptor consist of SEQ ID No.: 38.

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain, and
   wherein said member of the tumor necrosis factor receptor superfamily and said transmembrane domain are from DR3 and consist of SEQ ID No.: 41,
   wherein said cytoplasmic costimulatory signaling domain is CD28 and consists of SEQ ID No.: 35, and
   wherein said cytoplasmic T cell receptor signaling domain is CD3 zeta and consists of SEQ ID No.: 37.

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain, and
   wherein said artificial immune receptor comprises SEQ ID No.: 40.

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain, and
   wherein said artificial immune receptor consist of SEQ ID No.: 40.

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain, and
   wherein said member of the tumor necrosis factor receptor superfamily and said transmembrane domain are from LTBR and consist of SEQ ID No.: 33,
   wherein said cytoplasmic costimulatory signaling domain is CD28 and consists of SEQ ID No.: 35, and
   wherein said cytoplasmic T cell receptor signaling domain is CD3 zeta and consists of SEQ ID No.: 37.

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain, and
   wherein said artificial immune receptor comprises SEQ ID No.: 31.

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain, and
   wherein said artificial immune receptor consist of SEQ ID No.: 31.

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain, and
   wherein said member of the tumor necrosis factor receptor superfamily and said transmembrane domain are from CD40 and consist of SEQ ID No.: 46,
   wherein said cytoplasmic costimulatory signaling domain is CD28 and consists of SEQ ID No.: 35, and
   wherein said cytoplasmic T cell receptor signaling domain is CD3 zeta and consists of SEQ ID No.: 37.

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain, and
   wherein said artificial immune receptor comprises SEQ ID No.: 47.

In certain embodiments the present disclosure relates to an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain, and
   wherein said artificial immune receptor consist of SEQ ID No.: 47.

### Nucleic Acids, Vectors and Host Cells

The artificial immune receptors of the present disclosure are encoded by nucleic acids. Therefore, the in certain embodiments the present disclosure relates to nucleic acids encoding the artificial immune receptors.

In certain embodiments the present disclosure relates to a nucleic acid encoding an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain.

In certain embodiments the present disclosure relates to a nucleic acid encoding an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain, and
   wherein said member of the tumor necrosis factor receptor superfamily and said transmembrane domain are from TNFR2 and comprise SEQ ID No.: 39,
   wherein said cytoplasmic costimulatory signaling domain is CD28 and comprise SEQ ID No.: 35, and
   wherein said cytoplasmic T cell receptor signaling domain is CD3 zeta and comprise SEQ ID No.: 37.

In certain embodiments the present disclosure relates to a nucleic acid encoding an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain, and
   wherein said member of the tumor necrosis factor receptor superfamily and said transmembrane domain are from TNFR2 and consist of SEQ ID No.: 39,
   wherein said cytoplasmic costimulatory signaling domain is CD28 and consists of SEQ ID No.: 35, and
   wherein said cytoplasmic T cell receptor signaling domain is CD3 zeta and consists of SEQ ID No.: 37.

In certain embodiments the present disclosure relates to a nucleic acid encoding an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain, and
   wherein said artificial immune receptor comprises SEQ ID No.: 38.

In certain embodiments the present disclosure relates to a nucleic acid encoding an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain, and
   wherein said artificial immune receptor consist of SEQ ID No.: 38.

In certain embodiments the present disclosure relates to a nucleic acid encoding an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain, and
   wherein said member of the tumor necrosis factor receptor superfamily and said transmembrane domain are from DR3 and comprise SEQ ID No.: 41,
   wherein said cytoplasmic costimulatory signaling domain is CD28 and comprise SEQ ID No.: 35, and
   wherein said cytoplasmic T cell receptor signaling domain is CD3 zeta and comprise SEQ ID No.: 37.

In certain embodiments the present disclosure relates to a nucleic acid encoding an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain, and
   wherein said member of the tumor necrosis factor receptor superfamily and said transmembrane domain are from DR3 and consist of SEQ ID No.: 41,
   wherein said cytoplasmic costimulatory signaling domain is CD28 and consists of SEQ ID No.: 35, and
   wherein said cytoplasmic T cell receptor signaling domain is CD3 zeta and consists of SEQ ID No.: 37.

In certain embodiments the present disclosure relates to a nucleic acid encoding an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain, and
   wherein said artificial immune receptor comprises SEQ ID No.: 40.

In certain embodiments the present disclosure relates to a nucleic acid encoding an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain, and
   wherein said artificial immune receptor consist of SEQ ID No.: 40.

In certain embodiments the present disclosure relates to a nucleic acid encoding an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain, and
   wherein said member of the tumor necrosis factor receptor superfamily and said transmembrane domain are from LTBR and comprise SEQ ID No.: 33,
   wherein said cytoplasmic costimulatory signaling domain is CD28 and comprise SEQ ID No.: 35, and
   wherein said cytoplasmic T cell receptor signaling domain is CD3 zeta and comprise SEQ ID No.: 37.

In certain embodiments the present disclosure relates to a nucleic acid encoding an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain, and
   wherein said member of the tumor necrosis factor receptor superfamily and said transmembrane domain are from LTBR and consist of SEQ ID No.: 33,
   wherein said cytoplasmic costimulatory signaling domain is CD28 and consists of SEQ ID No.: 35, and
   wherein said cytoplasmic T cell receptor signaling domain is CD3 zeta and consists of SEQ ID No.: 37.

In certain embodiments the present disclosure relates to a nucleic acid encoding an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain, and
   wherein said artificial immune receptor comprises SEQ ID No.: 31.

In certain embodiments the present disclosure relates to a nucleic acid encoding an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain, and
   wherein said artificial immune receptor consist of SEQ ID No.: 31.

In certain embodiments the present disclosure relates to a nucleic acid comprising SEQ ID No.: 30.

In certain embodiments the present disclosure relates to a nucleic acid consisting of SEQ ID No.: 30.

In certain embodiments the present disclosure relates to a nucleic acid encoding an artificial immune receptor, comprising
a) an extracellular domain of a member of the tumor necrosis factor receptor superfamily;
b) a transmembrane domain,
c) a cytoplasmic costimulatory signaling domain,
d) a cytoplasmic T cell receptor signaling domain, and
   wherein said member of the tumor necrosis factor receptor superfamily and said transmembrane domain are from CD40 and comprise SEQ ID No.: 46,
   wherein said cytoplasmic costimulatory signaling domain is CD28 and comprise SEQ ID No.: 35, and
   wherein said cytoplasmic T cell receptor signaling domain is CD3 zeta and comprise SEQ ID No.: 37.

In certain embodiments the present disclosure relates to a nucleic acid encoding an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain, and
   wherein said member of the tumor necrosis factor receptor superfamily and said transmembrane domain are from CD40 and consist of SEQ ID No.: 46,
   wherein said cytoplasmic costimulatory signaling domain is CD28 and consists of SEQ ID No.: 35, and
   wherein said cytoplasmic T cell receptor signaling domain is CD3 zeta and consists of SEQ ID No.: 37.

In certain embodiments the present disclosure relates to a nucleic acid encoding an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain, and
   wherein said artificial immune receptor comprises SEQ ID No.: 47.

In certain embodiments the present disclosure relates to a nucleic acid encoding an artificial immune receptor, comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain, and
   wherein said artificial immune receptor consist of SEQ ID No.: 47.

In certain embodiments the present disclosure relates to a nucleic acid encoding a protein comprising SEQ ID No.: 30.

In certain embodiments the present disclosure relates to a nucleic acid encoding a protein consisting of SEQ ID No.: 30.

In certain embodiments the present disclosure relates a vector comprising a nucleic acid encoding an artificial immune receptor of the present disclosure.

In certain embodiments the present disclosure relates a vector comprising a nucleic acid encoding an artificial immune receptor comprising
a) an extracellular domain of a member of the tumor necrosis factor receptor superfamily;
b) a transmembrane domain,
c) a cytoplasmic costimulatory signaling domain, and
d) a cytoplasmic T cell receptor signaling domain.

In certain embodiments the present disclosure relates to a host cell comprising a nucleic acid or a vector encoding an artificial immune receptor according to the present disclosure.

In certain embodiments the present disclosure relates to a host cell expressing an artificial immune receptor of to the present disclosure.

In certain embodiments the present disclosure relates to a host cell expressing on the cell surface an artificial immune receptor of to the present disclosure.

In preferred embodiments, said host cell is a eukaryotic host cell. Therefore, in certain embodiments the present disclosure relates to a eukaryotic host cell comprising a nucleic acid or a vector encoding an artificial immune receptor according to the present disclosure. In other embodiments the present disclosure relates to a eukaryotic host cell expressing an artificial immune receptor of to the present disclosure. In yet other embodiments the present disclosure relates to a eukaryotic host cell expressing on the cell surface an artificial immune receptor of to the present disclosure.

### Therapeutic Use

The artificial immune receptors of the present disclosure can be used in therapeutically for the prevention and treatment of diseases and disorders.

In certain embodiments the present disclosure relates to artificial immune receptors as disclosed herein for use in medicine. In certain embodiments the present disclosure relates to artificial immune receptors as disclosed herein for use in the prevention of treatment of a disease or disorder. In certain embodiments said disease or disorder is an inflammatory disorder or cancer. In preferred embodiments said disease or disorder is an autoimmune disorder. In certain embodiments said disease or disorder is the treatment of graft versus host disease or the use in solid organ transplantation.

### CERTAIN SPECIFIC EMBODIMENTS

1. An artificial immune receptor, comprising
   a) an extracellular domain of a member of the tumor necrosis factor receptor superfamily,
   b) a transmembrane domain,
   c) a cytoplasmic costimulatory signaling domain, and
   d) a cytoplasmic T cell receptor signaling domain.
2. An artificial immune receptor according to claim 1, wherein said member of the tumor necrosis factor receptor superfamily is selected from TNFR1, TNFR2, Fas, DR4, DR5, DR3, DR6, EDAR, XEDAR, TROY, LTBR, NGFR, CD18, CD134, CD40, CD27, CD30, CD137, TRAILR3, TRAILR4, CD265, osteoprotegerin, CD266, TACI, BAFF, BAFF receptor, APRIL, CD270, CD269 and CD357.
3. An artificial immune receptor according to claim 1 or 2, wherein said member of the tumor necrosis factor receptor superfamily is selected from TNFR2, DR3, LTBR and CD40.
4. An artificial immune receptor according to any one of claims 1-3, wherein said transmembrane domain is from the same protein as the extracellular domain.
5. An artificial immune receptor according to any one of claims 1-4, wherein said member of the tumor necrosis factor receptor superfamily and said transmembrane domain are derived from TNFR2.
6. The artificial immune receptor according to claim 5, wherein artificial immune receptor comprises SEQ ID No.: 39.
7. The artificial immune receptor according to claim 5 or 6, wherein artificial immune receptor comprises SEQ ID No.: 38.
8. An artificial immune receptor according to any one of claims 1-4, wherein said member of the tumor necrosis factor receptor superfamily and said transmembrane domain are derived from DR3.
9. The artificial immune receptor according to claim 8, wherein artificial immune receptor comprises SEQ ID No.: 41.
10. The artificial immune receptor according to claim 8 or 9, wherein artificial immune receptor comprises SEQ ID No.: 40.
11. An artificial immune receptor according to any one of claims 1-4, wherein said member of the tumor necrosis factor receptor superfamily and said transmembrane domain are derived from LTBR.
12. The artificial immune receptor according to claim 11, wherein artificial immune receptor comprises SEQ ID No.: 33.
13. The artificial immune receptor according to claim 11 or 12, wherein artificial immune receptor comprises SEQ ID No.: 31.
14. An artificial immune receptor according to any one of claims 1-4, wherein said member of the tumor necrosis factor receptor superfamily and said transmembrane domain are derived from CD40.
15. The artificial immune receptor according to claim 11, wherein artificial immune receptor comprises SEQ ID No.: 46.
16. The artificial immune receptor according to claim 11 or 12, wherein artificial immune receptor comprises SEQ ID No.: 47.
17. An artificial immune receptor according to any one of claims 1-16, wherein said cytoplasmic costimulatory signaling domain is selected from 4-IBB (CD137), BAFFR, OX40, CD27, CD28, CD40, 2B4, GITR, HVEM, OX40, RELT, TACI, TROY, TWEAK, KIR receptors, TLR1 to TLR9 receptors, IL-2, IL-7 and IL-15 receptors.
18. An artificial immune receptor according to any one of claims 1-17, wherein said cytoplasmic costimulatory signaling domain is CD28.
19. An artificial immune receptor according to any one of claims 1-18, wherein said cytoplasmic costimulatory signaling domain comprises SEQ ID No.: 35.
20. An artificial immune receptor according to any one of claims 1-19, wherein said cytoplasmic T cell receptor signaling domain is selected from CD3 zeta, CD3 gamma and CD3 epsilon.
21. An artificial immune receptor according to any one of claims 1-20, wherein said cytoplasmic T cell receptor signaling domain is CD3 zeta.
22. An artificial immune receptor according to any one of claims 1-21, wherein said cytoplasmic T cell receptor signaling domain comprises SEQ ID No.: 37.
23. An artificial immune receptor according to any one of claims 1-7 or 17-22, wherein said member of the tumor necrosis factor receptor superfamily and said transmembrane domain are from TNFR2, said cytoplasmic costimulatory signaling domain is CD28 and said cytoplasmic T cell receptor signaling domain is CD3 zeta.
24. An artificial immune receptor according to any one of claims 1-7 or 17-23, wherein said member of the tumor necrosis factor receptor superfamily and said transmembrane domain are from TNFR2 and comprise SEQ ID No.: 39, said cytoplasmic costimulatory signaling domain is CD28 and comprise SEQ ID No.: 35, and said cytoplasmic T cell receptor signaling domain is CD3 zeta and comprise SEQ ID No.: 37.
25. An artificial immune receptor according to any one of claims 1-4, 8-10 or 17-22, wherein said member of the tumor necrosis factor receptor superfamily and said transmembrane domain are from DR3, said cytoplasmic costimulatory signaling domain is CD28 and said cytoplasmic T cell receptor signaling domain is CD3 zeta.
26. An artificial immune receptor according to any one of claims 1-4, 8-10, 17-22 or 25, wherein said member of the tumor necrosis factor receptor superfamily and said transmembrane domain are from DR3 and comprise SEQ ID No.: 41, said cytoplasmic costimulatory signaling domain is CD28 and comprise SEQ ID No.: 35, and said cytoplasmic T cell receptor signaling domain is CD3 zeta and comprise SEQ ID No.: 37.
27. An artificial immune receptor according to any one of claims 1-4, 11-13 or 17-22, wherein said member of the tumor necrosis factor receptor superfamily and said transmembrane domain are from LTBR, said cytoplasmic costimulatory signaling domain is CD28 and said cytoplasmic T cell receptor signaling domain is CD3 zeta.
28. An artificial immune receptor according to any one of claims 1-4, 11-13, 17-22 or 27, wherein said member of the tumor necrosis factor receptor superfamily and said transmembrane domain are from LTBR and comprise SEQ ID No.: 33, said cytoplasmic costimulatory signaling domain is CD28 and comprise SEQ ID No.: 35, and said cytoplasmic T cell receptor signaling domain is CD3 zeta and comprise SEQ ID No.: 37.
29. An artificial immune receptor according to any one of claims 1-4, 14-16 or 17-22, wherein said member of the tumor necrosis factor receptor superfamily and said transmembrane domain are from CD40, said cytoplasmic costimulatory signaling domain is CD28 and said cytoplasmic T cell receptor signaling domain is CD3 zeta.
30. An artificial immune receptor according to any one of claims 1-4, 14-16, 17-22 or 29, wherein said member of the tumor necrosis factor receptor superfamily and said transmembrane domain are from CD40 and comprise SEQ ID No.: 46, said cytoplasmic costimulatory signaling domain is CD28 and comprise SEQ ID No.: 35, and said cytoplasmic T cell receptor signaling domain is CD3 zeta and comprise SEQ ID No.: 37.
31. A nucleic acid encoding an artificial immune receptor according to any one of claims 1-30.
32. A vector comprising a nucleic acid according to claim 31.
33. A host cell comprising a nucleic acid according to claim 31 or a vector according to claim 32, or expressing an artificial immune receptor according to any one of claims 1-30.
34. An artificial immune receptor according to any one of claims 1-30 or a host cell of claim 33 for use in medicine.
35. An artificial immune receptor according to claim 34, wherein said use is use in the treatment of an inflammatory disorder.
36. An artificial immune receptor according to claim 35, wherein inflammatory disorder is graft versus host disease.
EXAMPLES

### Example 1: Materials

### Ethics Statement

Peripheral blood mononuclear cells for T-cell enrichment were isolated from leukocyte reduction chambers from healthy donors donating thrombocytes. Collection of immune cells from those donors was performed in compliance with the Helsinki Declaration after ethical approval by the local ethical committee and signed informed consent.

### Peripheral Blood Mononuclear Cell Isolation and Pre-Enrichment of Blood Lymphocytes

To isolate T cells from human blood, leukocyte reduction chambers (provided by Transfusion Medicine, University Clinics Regensburg) were used. Leukocytes were first diluted with PBS and the resulting blood and PBS mixture was split into four fractions and underlayed with an equal amount of Pancoll (PAN Biotech, Aidenbach, Germany). Samples were centrifuged at 1.000×g for 20 minutes at RT, with acceleration set to four and brake to zero. The PBMC layer was isolated and washed twice by centrifugation steps. Cells were pre-enriched with biotinylated anti-human CD25-PE (clone 2A3; BD Biosciences RRID: AB_341011) followed by column-based magnetic separation with anti-PE ultrapure microbeads (Miltenyi Biotec, Bergisch Gladbach, Germany) following manufacturer's protocol.

### Mice

Female BALB/c mice, C57BL/6 Nr4a1-eGFP mice (JAX stock #016617, C57BL/6-Tg(Nr4a1-EGFP/cre)820Khog/J) and C57BL/6 CD45.1+ mice (JAX stock #002014, B6.SJL-PtprcaPepcb/BoyCrl) were obtained from Charles River Breeding Laboratories (Wilmington, MA, USA) or the Jackson Laboratory (Bar Harbor, ME, USA). B6N.129(Cg)-Foxp3tm3Ayr mice (Foxp3.IRES-DTR/GFP) were bred to C57BL/6 CD45.1+ mice and served as bone marrow donors for GvHD experiments.

C57BL/6 Foxp3-hCD2 (Foxp3tm1(CD2/CD52)Shori) mice were a gift from S. Hori (P Natl Acad Sci USA. 2009; 106(6):1903-8). Mice were held under specific pathogen-free conditions and studies were approved by the Committee on Ethics of Animal Experiments at the Bavarian Government.

### Cell Lines

Phoenix-Eco and Phoenix-Ampho cell lines were purchased from ATCC (Cat #CRL-3214 and #CRL-3213). These are second-generation retrovirus producer cell lines for the generation of ecotropic and amphotropic retroviruses. EL4 lymphoblast cell line was purchased from ATCC (Cat #TIB-39).

Additional resources are listed in Tables 1, 2, 3 and 4.

TABLE 1
Antibodies

| Reagent or Resource | Source | Identifier |
|---|---|---|
| Antibodies | | |
| BV711 anti-mouse CD4 | Biolegend | RRID: AB_2562099 |
| PerCP/Cy5.5 anti-mouse CD4 | Biolegend | RRID: AB_100540 |
| BV605 anti-mouse CD4 | Biolegend | RRID: AB_100548 |
| BUV395 anti-mouse CD4 | BD Biosciences | RRID: AB_563790 |
| BUV737 anti-mouse CD4 | BD Biosciences | RRID: AB_564933 |
| FITC anti-mouse CD19 | Biolegend | RRID: AB_115506 |
| BV421 anti-mouse CD25 | Biolegend | RRID: AB_102034 |
| BV711 anti-mouse CD25 | Biolegend | RRID: AB_102049 |
| PerCP/Cy5.5 anti-mouse CD25 | Biolegend | RRID: AB_102030 |
| APC anti-mouse CD90.1 | Biolegend | RRID: AB_202526 |
| BV421 anti-mouse CD90.1 | Biolegend | RRID: AB_202529 |
| PerCp/Cy5.5 anti-mouse CD90.1 | Biolegend | RRID: AB_202516 |
| PE anti-mouse LTβR | Biolegend | RRID: AB_134403 |
| PE/Cy7 anti-mouse LTβR | Biolegend | RRID: AB_134409 |
| FITC anti-mouse CD69 | BD Biosciences | RRID: AB_553236 |
| PE anti-mouse TNFR2 | Biolegend | RRID: AB_113406 |
| PE anti-mouse DR3 | Biolegend | RRID: AB_144405 |
| BV421 anti-mouse LAP | Biolegend | RRID: AB_141408 |
| PE/Cy7 anti-mouse Tigit | Biolegend | RRID: AB_142108 |
| PE anti-mouse CD137 | Biolegend | RRID: AB_106106 |
| APC/Cy7 anti-mouse CD8a | Biolegend | RRID: AB_100714 |
| BV605 anti-mouse CD8a | Biolegend | RRID: AB_100738 |
| AF700 anti-mouse CD8a | Biolegend | RRID: AB_557959 |
| BV785 anti-mouse Klrg1 | Biolegend | RRID: AB_138429 |
| BV711 anti-mouse TCR-β-chain | Biolegend | RRID: AB_109243 |
| BUV737 anti-mouse CD45.1 | BD Biosciences | RRID: AB_612811 |
| BV605 anti-mouse CD45.1 | Biolegend | RRID: AB_110738 |
| BUV737 anti-mouse CD45.2 | BD Biosciences | RRID: AB_612778 |
| BUV496 anti-mouse H2Kb | BD Biosciences | RRID: AB_750257 |
| PE/Cy7 anti mouse H2Kd | Biolegend | RRID: AB_116622 |
| PE anti-mouse Nr4a1 | eBioscience | RRID: AB_12-5965-80 |
| PE anti-mouse Irf8 | eBioscience | RRID: AB_12-9852-82 |
| AF488 anti-mouse Foxp3 | eBioscience | RRID: AB_53-5773-82 |
| APC anti-mouse Foxp3 | eBioscience | RRID: AB_17-5773-82 |
| APC anti human CD2 | Biolegend | RRID: AB_300213 |
| PE anti-human CD2 | Biolegend | RRID: AB_300207/300208 |
| BV605 anti-human CD45RA | Biolegend | RRID: AB_304134 |
| BV711 anti-human CD4 | Biolegend | RRID: AB_317440 |
| BUV737 anti-human CD4 | BD Biosciences | RRID: AB_612749 |
| PE anti-human CD25 | BD Biosciences | RRID: AB_341011 |
| PE/Cy7 anti-human TCR-β-chain | Biolegend | RRID: AB_306720 |
| PerCP/Cy5.5 anti-human CD45RO | Biolegend | RRID: AB_304222 |
| APC anti-human CD127 | Biolegend | RRID: AB_351342 |
| BUV395 anti-human CCR8 | BD Biosciences | RRID: AB_747573 |
| BV711 anti-human CD137 | Biolegend | RRID: AB_309832 |
| PE/Cy7 anti-human GARP | Biolegend | RRID: AB_352507 |
| PE anti-human LTBR | Biolegend | RRID: AB_322008 |
| AF647 anti-human Foxp3 | Biolegend | RRID: AB_320114 |

TABLE 2
Chemicals, Plasmids and Recombinant Proteins

| Reagent or Resource | Source | Identifier |
|---|---|---|
| Antibodies | | |
| Recombinant human IL-2 | Novartis | Proleukin S ® 18 Mio U |
| TexMACS medium | Miltenyi Biotec | Cat#130-097-196 |
| Proteinase K | Carl Roth | Cat#7528.5 |
| HBSS | Gibco | Cat#14025092 |
| PBS | Gibco | Cat#10010023 |
| DPBS | Gibco | Cat#14190-094 |
| DMEM | Gibco | Cat#41965 |
| OptiMEM | ThermoFisherScientific | Cat#11058021 |
| Bovine Serum Albumin | Sigma | Cat#A4503 |
| Dnase I | Roche | Cat#11284932001 |
| TransIT-293 | MoBiTec | Cat#Mir2700 |
| Pancoll | PAN biotech | #P04-601000 |
| Non Essential Amino Acid Solution (100x) | PAN biotech | #P08-32100 |
| β-Mercaptoethanol | PAN biotech | #P07-05100 |
| L-Glutamin (100x) | PAN biotech | #P04-82050 |
| HEPES Buffer 1M | PAN biotech | #P05-011000 |
| Penicillin-Streptomycin (10,000 U/mL) | Gibco | #15140122 |
| soluble mLTBR-Ig fusion protein | T. Hehlgans | |
| pCL-Eco Retrovirus Packaging Vector | Novus Biologicals | NBP2-29540 |
| pCL-Ampho Retrovirus Packaging Vector | Novus Biologicals | NBP2-29541 |
| pMSCV-IRES-Thy1.1 DEST | Addgene (gift from | Cat#17442 |
| | Anjana Rao) | |

TABLE 3
Commercial Assays

| Reagent or Resource | Source | Identifier |
|---|---|---|
| Antibodies | | |
| Lamina Propria Dissociation Kit | Miltenyi Biotec | Cat#130-097-410 |
| GentleMACS C tube | Miltenyi | Cat#130-096-334 |
| Anti-CD2 microbeads, human | Miltenyi Biotec | Cat#130-091-114 |
| Anti-PE microbeads, ultrapure | Miltenyi Biotec | Cat#130-105-639 |
| LS column | Miltenyi Biotec | Cat#130-042-401 |
| MS column | Miltenyi Biotec | Cat#130-042-201 |
| ACK lysis buffer | Gibco | Cat#A1049201 |
| Foxp3/Transcription Factor Buffer Set | eBiosciences | Cat#00-5523-00 |
| RNEasy Plus Mini Kit | Quiagen | Cat#74134 |
| round bottom 96 well plate tc treated | Corning | Cat#3799 |
| Mouse T cell activation and expansion kit | Miltenyi Biotec | Cat#130-093-627 |
| T Cell TransAct ™, human | Miltenyi Biotec | Cat#130-111-160 |
| CellTrace ™ CFSE Cell Proliferation Kit | Thermo Fisher/ | Cat#V12883 |
| | Life Technologies | |
| Qiagen Rneasy Micro Kit | Qiagen | Cat#74004 |
| High-Sensitivity RNA screentape | Agilent | Cat#5067-5579 |
| SMART-seq Stranded Kit | Takara | Cat# 634444 |

TABLE 4
Software and algorithms

| Software | Source | Identifier |
|---|---|---|
| Flowjo (V10.6.2) | BD Biosciences | https://www.flowjo.com/ |
| | | solutions/flowjo/downloads/ |
| FACS Diva (V8.0.2) | BD Biosciences | http://www.bdbiosciences.com/en-us/ |
| | | instruments/research-instruments/ |
| | | research-software/flow-cytometry- |
| | | acquisition/facsdiva-software |
| GraphPad Prism (V8.4.3) | GraphPad Software Inc | https://www.graphpad.com/ |
| SnapGene (V1.4.1) | GSL Biotech LLC | https://www.snapgene.com/ |

### Statistical Analysis

Data were analyzed with Prism software or algorithm. Statistical details are indicated in the figure legend. For survival differences Kaplan-Meier analysis was performed and the log-rank test was used. P values <0.05 were considered significant (*p<0.05; **p<0.01; ***p<0.001).

### Example 2: Methods

### Digestion of Murine Tissues for Flow Cytometric Analysis, FACS Sorting of Cells and Transplantation

To isolate cells from colon tissue, colons were isolated, cleared of feces and prepared according to manufacturer's instructions with a lamina propria dissociation kit (Miltenyi Biotec, Bergisch Gladbach, Germany) and a gentle MACS device (Miltenyi Biotec, Bergisch Gladbach, Germany, program 37C_mLPDK_1). More detailed protocols about T cell isolation from murine tissues are published (Eur J Immunol. 2019; 49(10):1457-973.).

To isolate cells from blood, peripheral blood was collected and incubated in heparin buffer. Blood samples were centrifuged and red blood cells were lysed using ACK lysis buffer (ThermoFisher/GIBCO, Waltham, MA, USA, #A1049201).

To isolate cells from spleen and lymph nodes, tissues were harvested and mechanically disintegrated. Samples were centrifuged and red blood cells were lysed using ACK lysis buffer. Samples were pre-enriched with anti-human CD2 (RPA-2.10) microbeads (for Foxp3-hCD2 reporter mice) or anti-CD25 (PC61) antibody staining and anti-PE microbeads.

To isolate cells from bone marrow for transplantation, femurs were collected and femoral head and femoral medial and lateral epicondyle were removed. Bone shafts were flushed with PBS and bone marrow collected by centrifugation followed by red blood cell lysis.

### Preparation of Samples for Flow Cytometry

Single cells suspensions were prepared and pre-enriched as described previously (Eur J Immunol. 2019; 49(10):1457-973). Samples were stained either in 1.5 mL Eppendorf tubes or 96-well plates in FACS buffer (1% FCS in PBS). Surface staining was performed at 4° C. for 20 minutes in 50-100 μL staining volume. Antibodies were used, if not indicated otherwise, as recommended by the manufacturer. Following antibodies were used for surface staining of murine samples: TCR-β-chain (H57-597), CD4 (RM4-5), CD8a (53-6.7), CD19 (6D5), CD25 (PC61), CD45.1 (A20), CD45.2 (104), CD90.1(OX-7), CD120b (TR79-89), I-A/I-E/MHCII (M5/114.15.2), KIrg1 (2F1), Tigit (1G9), DR3 (4C12), CD62L (MEL-14), CD137 (I7B5), CD69 (H1.2F3), LTBR (5G11), LAP (TW7-16B4), H2Kb (AF6-88.5), H2Kd (SF1-1.1), hCD2 (RPA-2.10).

The following antibodies were used for surface staining of human samples: CD4 (OKT4), CD25 (BC96, 2A3), CD127 (A019D5), CD45RO (UCHL1), CD45RA (HI100), CD137 (41BB), TCR-β chain (IP26), CCR8 (433H RUO), and GARP (7B11).

Intracellular staining was performed with the Foxp3/Transcription Factor Buffer Set (Thermo Fisher/Biosciences, Waltham, MA, USA) according to manufacturer's protocol with the following adaptations: intracellular staining steps were performed for 60 minutes at room temperature. Antibodies for intracellular staining include Foxp3 (JFK-16S), Nr4a1 (12.14), Irf8 (V3GYWCH) for mouse and Foxp3 (206D) for human samples. Dead cells were excluded with a fixable live/dead dye (eBioscience Fixable Viability Dye eFluor780). All antibodies are listed in the resource table.

Flow Cytometry and FACS Sorting of T Cells from Blood, Tissues and Cell Cultures

Cells were isolated, pre-enriched and stained as described previously (Eur J Immunol. 2019; 49(10): 1457-973). Afterwards, samples were filtered with a 40 μM filter unit and acquired on a BD FACSymphony™, a BD FACSCelesta™ or a BD FACSFusion™ flow cytometer (all from Becton Dickinson, Franklin Lakes/NJ, USA). BD CS&T beads were used to validate machine functionality. Fluorescence spillover compensation was performed with lymphocytes stained with anti-CD4 antibodies in the respective colors. Flow cytometry data were analyzed using BD FlowJo™ (Version 10.6.2). Sorting was performed with a BD FACSAriall™ or BD FACSFusion™ cell sorter with 70 μm nozzle. Post-sort quality controls were performed as applicable. For murine Treg cultures, anti-CD25 enriched cells were sorted for naïve CD4+CD25+CD62L+ Treg (Leukemia 2020; 34(3):895-908). hCD2 pre-enriched cells were sorted for CD4+CD25+hCD2+. For cultivation, cells were sorted directly into cell culture medium. All procedures were performed in DNA low-bind tubes (Eppendorf, Hamburg, Germany, #0030108051) or 15 ml tubes.

For bulk RNA sequencing 4×104-8×10⁴ cells cells were sorted on live CD4+CD25+CD90.1+GFP(Nr4a1)+LAP+ orCD4+CD25+CD90.1 directly into 500 μL RLT+ lysis buffer (Qiagen, Hilden, Germany, RNEasy Plus Micro Kit #74034). For human Treg cultures, CD25 pre-enriched cells from human blood were sorted on TCR-ß-chain+CD4+CD25+CD45RA+ directly into TexMACS medium.

### Murine and Human Treg Cell Cultures

For murine Treg cultures, sorted Treg cells were seeded at 3×10⁴-4×10⁴ cells per well in a 96 well round-bottom plate (Corning, Wiesbaden, Germany) with anti-CD3/CD28 beads (Miltenyi Biotec, Bergisch Gladbach, Germany, 4 beads per cell) and 2000 U/ml rhIL-2 (Proleukin S®, Novartis, Basel, Switzerland). Culture medium was DMEM (Gibco/Invitrogen) supplemented with 10% FCS, 2 mM I-glutamine, 5×10-5 M 2-ME, 10 mM HEPES, 1% NEAA (PAN Biotech, Aidenbach, Germany), 100 U/ml penicillin-streptomycin (Gibco/Sigma-Aldrich, St. Louis/MO, USA). Murine Treg cells were transduced 48 h after isolation. Human sorted Treg cells were cultured in TexMACS medium supplemented with 500 U/ml rhIL-2 and TransAct (1:100 diluted). Human Treg cells were transduced 48 h after isolation.

### Retroviral Transduction of Treg Cells

Retrovirus in the pMSCV-Thy1.1 system can be manufactured in Phoenix-Eco cells, a pCLEco (packaging plasmid) carrying variant of HEK293 cells. Therefore, Phoenix-Eco cells were seeded on a gelatin matrix at 1.8×10⁶ cells per well in a six well plate 6 hours before lipofection. To produce liposomal particles containing the viral transgene, 3 μg of vector DNA and 1 μg of additional pCL-Eco packaging plasmid were coincubated with 12 μL of TransIT-293 transfection reagent (MoBiTec, Göttingen, Germany) for 20 minutes in OptiMEM medium at RT. Liposomes were added to Phoenix-Eco cells and incubated for an additional 16 hours. Afterwards medium of was exchanged and production of viral particles could proceed for 24 h. Then supernatant with produced pMSCV retrovirus was added to Treg cell cultures and mixed gently. Treg cells were transduced for 6.5 hours incubation at 37° C. Afterwards, viral supernatant was removed and cells were incubated with fresh medium supplemented with IL-2 (2000 U/ml) for another 72 to 96 hours. Then, cells were harvested and anti-CD3/CD28 antibody coupled beads were removed by using a MACSiMAG separator magnet (Miltenyi Biotec, Bergisch Gladbach, Germany). For transduction of human Tregs an amphotrophic Phoenix cell line was used. Phoenix-Ampho cells were seeded at 2×10⁶ cells per well in a six well plate 6 hours before lipofection. For production of liposomal particles containing the viral transgene, 3 μg of vector DNA and 1 μg of additional pCL-Ampho packaging plasmid were coincubated with 12 μL of TransIT-293 transfection reagent (MoBiTec, Göttingen, Germany) for 20 minutes in OptiMEM medium at RT. Liposomes were added to Phoenix-Ampho cells and incubated for an additional 16 hours. Afterwards medium of was exchanged and production of viral particles could proceed for 24 h. Then supernatant with produced pMSCV retrovirus was added to Treg cell cultures and mixed gently. Treg cells were transduced for 6.5 hours incubation at 37° C. Afterwards, viral supernatant was removed and cells were incubated with fresh medium supplemented with IL-2 (500 U/ml), 100 U/ml penicillin-streptomycin and fresh TransAct (1:100 diluted) for another 72 to 96 hours.

Transient Transfection of HEK293 Cells with Ligands of the TNFSF

For production of liposomal particles, 2 μg of plasmid DNA was incubated with 6 μl TransIT-293 transfection reagent in 100 μl OptiMEM medium for 20 minutes at room temperature. Then liposomes were gently added to 5×105 HEK293 cells resuspended in 500 μl DMEM. After shaking for 1 minute 230 μl DMEM were added and 100 μl of the cell-liposome suspension (containing approximately 6×10⁴ cells) were added per well into a 96 well flat bottom plate. 18 hours after lipofection medium was changed and transfected HEK293 cells could be used for coculture experiments.

### Treg Proliferation Assay

Anti-CD3/CD28 beads were removed from transduced Treg cultures via a MACSiMAG separator magnet. Tregs were rested for 18 hours in fresh medium supplemented with 100 U/ml rhIL-2. Then Tregs were labeled with CellTrace™ CFSE Cell Proliferation dye (1 μM) and added onto a HEK293 cells expressing mTNF, mTL1A or mLIGHT on the cell surface. rhIL-2 (2000 U/ml) was added to the cell cultures. After 72 h of coincubation proliferation of transduced CFSE-labeled Tregs was analyzed via flow cytometry.

### RNA-Sequencing

Total RNA was isolated using the Qiagen Rneasy Micro Kit (Qiagen, Hilden, Germany) and RNA was eluted in 14 μL RNAse-free water. RNA quality was assessed using the Tapestation system 4200 and High-Sensitivity RNA screentape (Agilent). 7 μl of the RNA was used for generating RNA-seq libraries using the SMART-seq Stranded Kit from Takara (Mountain View/CA, USA). Indexed libraries were pooled in an equimolar ratio and sequenced on an Illumina NextSeq 550 machine with NextSeq 500/550 High Output Kit v2.5 (75 cycles).

### GvHD Model

BALB/c (H2Kd) recipients were irradiated (8Gy) and transplanted retrobulbar with 2.5×10⁶ BM cells with or without (BM control) 5×10⁵ splenocytes from C57BL/6 donors (H2Kb, congenic CD45.1). The animals in the therapy groups received 2.5×10⁵ in vitro expanded and transduced Treg (C57BL/6, Foxp3-hCD2, congenic CD45.2). Recipients were monitored daily, body weight and GvHD symptoms assessed two or three times weekly by nonblinded investigators applying standardized scoring.

### Example 3: Recombinant Proteins

### Murine TNF

For the production of murine TNF the expression plasmid BCMGS-L6 coding for a non-cleavable mouse TNF protein was used (JBC 1999, 274(53):38112-8).

### Murine TL1A

To generate a non-cleavable version of TL1A (mTL1A), amino acids 69-93 were deleted from wildtype sequence as described in J Immunol 2018; 200(4):1360-9. cDNA encoding the non-cleavable mTL1A was cloned into pIRES2-DsRed via NheI/SalI. The nucleotide sequence of mTL1A is depicted below:

(SEQ ID NO.: 1)
ATGGGGGGCTCTCTGGTCAGAAGGGATCAGAAGTCTCTCCAAGACAGCAGAAGGATGGCAGAGGAGCT
GGGGTTGGGCTTCGGAGAAGGAGTCCCAGTGGAAGTGCTGCCGGAAGGCTGTAGACACAGGCCAGAGG
CCAGGGCCGGGCTAGCTGCCAGGAGCAAAGCCTGCCTGGCTCTCACCTGCTGCCTGTTGTCATTTCCC
ATCCTCGCAGGACTTAGCACCCTCCTAATGGCTGGCCAGCTCCGGGTCCCCGGAGGCAAGCCGAGAGC
ACACCTGACAATTAAGAAACAAACCCCAGCACCACATCTGAAAAATCAGCTCTCTGCTCTACACTGGG
AACATGACCTAGGGATGGCCTTCACCAAGAACGGGATGAAGTACATCAACAAATCCCTGGTGATCCCA
GAGTCAGGAGACTATTTCATCTACTCCCAGATCACATTCCGAGGGACCACATCTGTGTGTGGTGACAT
CAGTCGGGGGAGACGACCAAACAAGCCAGACTCCATCACCATGGTTATCACCAAGGTAGCAGACAGCT
ACCCTGAGCCTGCCCGCCTACTAACAGGGTCCAAGTCTGTGTGTGAAATAAGCAACAACTGGTTCCAG
TCCCTCTACCTTGGGGCCACGTTCTCCTTGGAAGAAGGAGACAGACTAATGGTAAACGTCAGTGACAT
CTCCTTGGTGGATTACACAAAAGAAGATAAAACTTTCTTTGGAGCTTTCTTGCTATAA

### Human LIGHT

cDNA encoding human LIGHT was cloned into pcDNA3.1 neo via BamHI/EcoRV. The nucleotide sequence of hLIGHT is depicted below:

(SEQ ID NO.: 2)
ATGGAGGAGAGTGTCGTACGGCCCTCAGTGTTTGTGGTGGATGGACAGACCGACATCCCATTCACGAG
GCTGGGACGAAGCCACCGGAGACAGTCGTGCAGTGTGGCCCGGGTGGGTCTGGGTCTCTTGCTGTTGC
TGATGGGGGCCGGGCTGGCCGTCCAAGGCTGGTTCCTCCTGCAGCTGCACTGGCGTCTAGGAGAGATG
GTCACCCGCCTGCCTGACGGACCTGCAGGCTCCTGGGAGCAGCTGATACAAGAGCGAAGGTCTCACGA
GGTCAACCCAGCAGCGCATCTCACAGGGGCCAACTCCAGCTTGACCGGCAGCGGGGGGCCGCTGTTAT
GGGAGACCCAGCTGGGCCTGGCCTTCCTGAGGGGCCTCAGCTACCACGATGGGGCCCTTGTGGTCACC
AAAGCTGGCTACTACTACATCTACTCCAAGGTGCAGCTGGGCGGTGTGGGCTGCCCGCTGGGCCTGGC
CAGCACCATCACCCACGGCCTCTACAAGCGCACACCCCGCTACCCCGAGGAGCTGGAGCTGTTGGTCA
GCCAGCAGTCACCCTGCGGACGGGCCACCAGCAGCTCCCGGGTCTGGTGGGACAGCAGCTTCCTGGGT
GGTGTGGTACACCTGGAGGCTGGGGAGGAGGTGGTCGTCCGTGTGCTGGATGAACGCCTGGTTCGACT
GCGTGATGGTACCCGGTCTTACTTCGGGGCTTTCATGGTGTGA

### Murine LIGHT

cDNA encoding mouse LIGHT was cloned into pIRES2-DsRed via NheI/SalI. Amino acids 63-84 were deleted in order to generate an uncleavable form of mouse LIGHT. The nucleotide sequence of mLIGHT is depicted below:

(SEQ ID NO.: 3)
ATGGAGAGTGTGGTACAGCCTTCAGTGTTTGTGGTGGATGGACAGACGGACATCCCATTCAGGCGGCT
GGAACAGAACCACCGGAGACGGCGCTGTGGCACTGTCCAGGTCAGCCTGGCCCTGGTGCTGCTGCTAG
GTGCTGGGCTGGCCACTCAGGGCTGGTTTCTCCTGAGACTGCATCAACGTCAACGATCTCACCAGGCC
AACCCAGCAGCACATCTTACAGGAGCCAACGCCAGCTTGATAGGTATTGGTGGACCTCTGTTATGGGA
GACACGACTTGGCCTGGCCTTCTTGAGGGGCTTGACGTATCATGATGGGGCCCTGGTGACCATGGAGC
CCGGTTACTACTATGTGTACTCCAAAGTGCAGCTGAGCGGCGTGGGCTGCCCCCAGGGGCTGGCCAAT
GGCCTCCCCATCACCCATGGACTATACAAGCGCACATCCCGCTACCCGAAGGAGTTAGAACTGCTGGT
CAGTCGGCGGTCACCCTGTGGCCGGGCCAACAGCTCCCGAGTCTGGTGGGACAGCAGCTTCCTGGGCG
GCGTGGTACATCTGGAGGCTGGGGAAGAGGTGGTGGTCCGCGTGCCTGGAAACCGCCTGGTCAGACCA
CGTGACGGCACCAGGTCCTATTTCGGAGCTTTCATGGTCTGA

### Example 4: Design of Artificial Immune Receptors (AIRs)

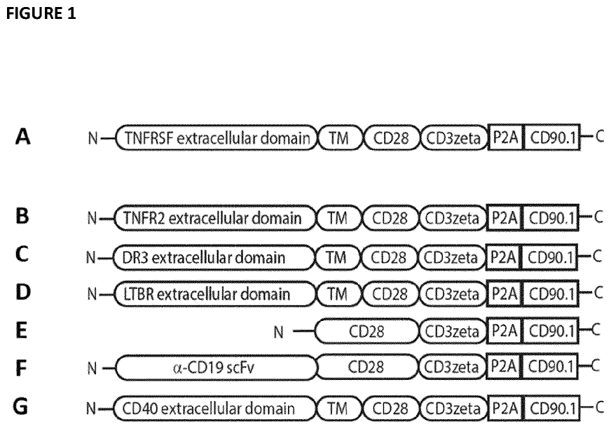

Murine and human AIRs were designed based on published nucleotide sequences of the individual parts and domains (https://www.ensembl.org/). Extracellular and transmembrane domains of LTbR, DR3, TNFR2 and CD40 were fused to the intracellular signaling domains of CD28 and the CD3-zeta-chain. Two point mutations were introduced into murine CD3-zeta-chain as they were shown to increase expression of chimeric antigen receptors (Blood 2003; 102(13):4320-5). As negative control, an ORF was used coding for transmembrane and signaling domain of CD28 and the signaling domain of the CD3-zeta-chain. The anti-CD19 CAR as well as the anti-CEA CAR have been described earlier (Blood 2010; 116(19):3875-86; Mol Ther 2019; 27(10):1825-35). The cDNAs coding AIRs as well as the anti-CD19 CAR and the anti-CEA CAR were fused to the congenic marker CD90.1 by ligating to a self cleaving P2A sequence. cDNAs were synthesized by ThermoFisher/Life Technologies (Waltham, MA, USA) and cloned into the pMSCV-Thy1.1 retroviral backbone (Addgene, cat #17442) via NotI/Mlu. An overview of the constructs is shown in FIG. 1. Sequences of the constructs and their parts are shown in Table 5 (N=nucleic acid, P=polypeptide). The CD90.1 marker gene is cut off via the P2A cleavage site and is not part of the AIR.

TABLE 5

| SEQ ID | | | |
|---|---|---|---|
| No. | Description | N/P | Sequence |
| 4 | TNFR2, complete | N | ATGGCGCCCGCCGCCCTCTGGGTCGCGCTGGTCTTCGAACT |
| | AIR, mouse | | GCAGCTGTGGGCCACCGGGCACACAGTGCCCGCCCAGGTTG |
| | | | TCTTGACACCCTACAAACCGGAACCTGGGTACGAGTGCCAG |
| | | | ATCTCACAGGAATACTATGACAGGAAGGCTCAGATGTGCTG |
| | | | TGCTAAGTGTCCTCCTGGCCAATATGTGAAACATTTCTGCA |
| | | | ACAAGACCTCGGACACCGTGTGTGCGGACTGTGAGGCAAGC |
| | | | ATGTATACCCAGGTCTGGAACCAGTTTCGTACATGTTTGAG |
| | | | CTGCAGTTCTTCCTGTACCACTGACCAGGTGGAGATCCGCG |
| | | | CCTGCACTAAACAGCAGAACCGAGTGTGTGCTTGCGAAGCT |
| | | | GGCAGGTACTGCGCCTTGAAAACCCATTCTGGCAGCTGTCG |
| | | | ACAGTGCATGAGGCTGAGCAAGTGCGGCCCTGGCTTCGGAG |
| | | | TGGCCAGTTCAAGAGCCCCAAATGGAAATGTGCTATGCAAG |
| | | | GCCTGTGCCCCAGGGACGTTCTCTGACACCACATCATCCAC |
| | | | TGATGTGTGCAGGCCCCACCGCATCTGTAGCATCCTGGCTA |
| | | | TTCCCGGAAATGCAAGCACAGATGCAGTCTGTGCGCCCGAG |
| | | | TCCCCAACTCTAAGTGCCATCCCAAGGACACTCTACGTATC |
| | | | TCAGCCAGAGCCCACAAGATCCCAACCCCTGGATCAAGAGC |
| | | | CAGGGCCCAGCCAAACTCCAAGCATCCTTACATCGTTGGGT |
| | | | TCAACCCCCATTATTGAACAAAGTACCAAGGGTGGCATCTC |
| | | | TCTTCCAATTGGTCTGATTGTTGGAGTGACATCACTGGGTC |
| | | | TGCTGATGTTAGGACTGGTGAACTGCATCATCCTGGTGCAG |
| | | | AGGAACAGCAGAAGAAACAGAGGCGGCCAGAGCGACTACAT |
| | | | GAACATGACCCCCAGAAGGCCAGGCCTGACCAGAAAGCCCT |
| | | | ACCAGCCCTACGCCCCTGCCAGAGACTTCGCCGCCTACAGA |
| | | | CCCAGAGCCAAGTTCAGCAGATCCGCCGAGACAGCCGCCAA |
| | | | CCTGCAGGATCCCAACCAGCTGTACAACGAGCTGAACCTGG |
| | | | GCAGACGGGAGGAATACGACGTGCTGGAAAAGAAGAGAGCC |
| | | | AGGGACCCCGAGATGGGCGGCAAGCAGCAGAGAAGAAGAAA |
| | | | CCCTCAGGAAGGCGTCTACAACGCCCTGCAGAAAGACAAGA |
| | | | TGGCCGAGGCCTACAGCGAGATCGGCACCAAGGGCGAGAGA |
| | | | AGAAGGGGCAAGGGCCACGATGGCCTGTACCAGGGCCTGTC |
| | | | CACCGCCACCAAGGACACCTACGACGCCCTGCACATGCAGA |
| | | | CCCTGGCCCCCAGAGCCACGAACTTCTCTCTGTTAAAGCAA |
| | | | GCAGGAGACGTGGAAGAAAACCCCGGTCCTATGAACCCAGC |
| | | | CATCAGCGTCGCTCTCCTGCTCTCAGTCTTGCAGGTGTCCC |
| | | | GAGGGCAGAAGGTGACCAGCCTGACAGCCTGCCTGGTGAAC |
| | | | CAAAACCTTCGCCTGGACTGCCGCCATGAGAATAACACCAA |
| | | | GGATAACTCCATCCAGCATGAGTTCAGCCTGACCCGAGAGA |
| | | | AGAGGAAGCACGTGCTCTCAGGCACCCTTGGGATACCCGAG |
| | | | CACACGTACCGCTCCCGCGTCACCCTCTCCAACCAGCCCTA |
| | | | TATCAAGGTCCTTACCCTAGCCAACTTCACCACCAAGGATG |
| | | | AGGGCGACTACTTTTGTGAGCTTCGCGTCTCGGGCGCGAAT |
| | | | CCCATGAGCTCCAATAAAAGTATCAGTGTGTATAGAGACAA |
| | | | GCTGGTCAAGTGTGGCGGCATAAGCCTGCTGGTTCAGAACA |
| | | | CATCCTGGATGCTGCTGCTGCTGCTTTCCCTCTCCCTCCTC |
| | | | CAAGCCCTGGACTTCATTTCTCTGTGA |
| 5 | TNFR2, complete | P | MAPAALWVALVFELQLWATGHTVPAQVVLTPYKPEPGYECQ |
| | AIR, mouse | | ISQEYYDRKAQMCCAKCPPGQYVKHFCNKTSDTVCADCEAS |
| | | | MYTQVWNQFRTCLSCSSSCTTDQVEIRACTKQQNRVCACEA |
| | | | GRYCALKTHSGSCRQCMRLSKCGPGFGVASSRAPNGNVLCK |
| | | | ACAPGTFSDTTSSTDVCRPHRICSILAIPGNASTDAVCAPE |
| | | | SPTLSAIPRTLYVSQPEPTRSQPLDQEPGPSQTPSILTSLG |
| | | | STPIIEQSTKGGISLPIGLIVGVTSLGLLMLGLVNCIILVQ |
| | | | RNSRRNRGGQSDYMNMTPRRPGLTRKPYQPYAPARDFAAYR |
| | | | PRAKFSRSAETAANLQDPNQLYNELNLGRREEYDVLEKKRA |
| | | | RDPEMGGKQQRRRNPQEGVYNALQKDKMAEAYSEIGTKGER |
| | | | RRGKGHDGLYQGLSTATKDTYDALHMQTLAPRATNFSLLKQ |
| | | | AGDVEENPGPMNPAISVALLLSVLQVSRGQKVTSLTACLVN |
| | | | QNLRLDCRHENNTKDNSIQHEFSLTREKRKHVLSGTLGIPE |
| | | | HTYRSRVTLSNQPYIKVLTLANFTTKDEGDYFCELRVSGAN |
| | | | PMSSNKSISVYRDKLVKCGGISLLVQNTSWMLLLLLSLSLL |
| | | | QALDFISL |
| 6 | TNFR2, | N | ATGGCGCCCGCCGCCCTCTGGGTCGCGCTGGTCTTCGAACT |
| | extracellular and | | GCAGCTGTGGGCCACCGGGCACACAGTGCCCGCCCAGGTTG |
| | transmembrane | | TCTTGACACCCTACAAACCGGAACCTGGGTACGAGTGCCAG |
| | domain, mouse | | ATCTCACAGGAATACTATGACAGGAAGGCTCAGATGTGCTG |
| | | | TGCTAAGTGTCCTCCTGGCCAATATGTGAAACATTTCTGCA |
| | | | ACAAGACCTCGGACACCGTGTGTGCGGACTGTGAGGCAAGC |
| | | | ATGTATACCCAGGTCTGGAACCAGTTTCGTACATGTTTGAG |
| | | | CTGCAGTTCTTCCTGTACCACTGACCAGGTGGAGATCCGCG |
| | | | CCTGCACTAAACAGCAGAACCGAGTGTGTGCTTGCGAAGCT |
| | | | GGCAGGTACTGCGCCTTGAAAACCCATTCTGGCAGCTGTCG |
| | | | ACAGTGCATGAGGCTGAGCAAGTGCGGCCCTGGCTTCGGAG |
| | | | TGGCCAGTTCAAGAGCCCCAAATGGAAATGTGCTATGCAAG |
| | | | GCCTGTGCCCCAGGGACGTTCTCTGACACCACATCATCCAC |
| | | | TGATGTGTGCAGGCCCCACCGCATCTGTAGCATCCTGGCTA |
| | | | TTCCCGGAAATGCAAGCACAGATGCAGTCTGTGCGCCCGAG |
| | | | TCCCCAACTCTAAGTGCCATCCCAAGGACACTCTACGTATC |
| | | | TCAGCCAGAGCCCACAAGATCCCAACCCCTGGATCAAGAGC |
| | | | CAGGGCCCAGCCAAACTCCAAGCATCCTTACATCGTTGGGT |
| | | | TCAACCCCCATTATTGAACAAAGTACCAAGGGTGGCATCTC |
| | | | TCTTCCAATTGGTCTGATTGTTGGAGTGACATCACTGGGTC |
| | | | TGCTGATGTTAGGACTGGTGAACTGCATCATCCTGGTGCAG |
| | | | AGG |
| 7 | TNFR2, | P | MAPAALWVALVFELQLWATGHTVPAQVVLTPYKPEPGYECQ |
| | extracellular and | | ISQEYYDRKAQMCCAKCPPGQYVKHFCNKTSDTVCADCEAS |
| | transmembrane | | MYTQVWNQFRTCLSCSSSCTTDQVEIRACTKQQNRVCACEA |
| | domain, mouse | | GRYCALKTHSGSCRQCMRLSKCGPGFGVASSRAPNGNVLCK |
| | | | ACAPGTFSDTTSSTDVCRPHRICSILAIPGNASTDAVCAPE |
| | | | SPTLSAIPRTLYVSQPEPTRSQPLDQEPGPSQTPSILTSLG |
| | | | STPIIEQSTKGGISLPIGLIVGVTSLGLLMLGLVNCIILVQ |
| | | | R |
| 8 | CD28 signaling | N | AACAGCAGAAGAAACAGAGGCGGCCAGAGCGACTACATGAA |
| | domain, mouse | | CATGACCCCCAGAAGGCCAGGCCTGACCAGAAAGCCCTACC |
| | | | AGCCCTACGCCCCTGCCAGAGACTTCGCCGCCTACAGACCC |
| 9 | CD28 signaling | P | NSRRNRGGQSDYMNMTPRRPGLTRKPYQPYAPARDFAAYRP |
| | domain, mouse | | |
| 10 | CD3 zeta domain, | N | AGAGCCAAGTTCAGCAGATCCGCCGAGACAGCCGCCAACCT |
| | mouse | | GCAGGATCCCAACCAGCTGTACAACGAGCTGAACCTGGGCA |
| | | | GACGGGAGGAATACGACGTGCTGGAAAAGAAGAGAGCCAGG |
| | | | GACCCCGAGATGGGCGGCAAGCAGCAGAGAAGAAGAAACCC |
| | | | TCAGGAAGGCGTCTACAACGCCCTGCAGAAAGACAAGATGG |
| | | | CCGAGGCCTACAGCGAGATCGGCACCAAGGGCGAGAGAAGA |
| | | | AGGGGCAAGGGCCACGATGGCCTGTACCAGGGCCTGTCCAC |
| | | | CGCCACCAAGGACACCTACGACGCCCTGCACATGCAGACCC |
| | | | TGGCCCCCAGA |
| 11 | CD3 zeta domain, | P | RAKFSRSAETAANLQDPNQLYNELNLGRREEYDVLEKKRAR |
| | mouse | | DPEMGGKQQRRRNPQEGVYNALQKDKMAEAYSEIGTKGERR |
| | | | RGKGHDGLYQGLSTATKDTYDALHMQTLAPR |
| 12 | P2A cleavage site | N | GCCACGAACTTCTCTCTGTTAAAGCAAGCAGGAGACGTGGA |
| | | | AGAAAACCCCGGTCCT |
| 13 | P2A cleavage site | P | ATNFSLLKQAGDVEENPGP |
| 14 | CD90.1 marker, | N | ATGAACCCAGCCATCAGCGTCGCTCTCCTGCTCTCAGTCTT |
| | mouse | | GCAGGTGTCCCGAGGGCAGAAGGTGACCAGCCTGACAGCCT |
| | | | GCCTGGTGAACCAAAACCTTCGCCTGGACTGCCGCCATGAG |
| | | | AATAACACCAAGGATAACTCCATCCAGCATGAGTTCAGCCT |
| | | | GACCCGAGAGAAGAGGAAGCACGTGCTCTCAGGCACCCTTG |
| | | | GGATACCCGAGCACACGTACCGCTCCCGCGTCACCCTCTCC |
| | | | AACCAGCCCTATATCAAGGTCCTTACCCTAGCCAACTTCAC |
| | | | CACCAAGGATGAGGGCGACTACTTTTGTGAGCTTCGCGTCT |
| | | | CGGGCGCGAATCCCATGAGCTCCAATAAAAGTATCAGTGTG |
| | | | TATAGAGACAAGCTGGTCAAGTGTGGCGGCATAAGCCTGCT |
| | | | GGTTCAGAACACATCCTGGATGCTGCTGCTGCTGCTTTCCC |
| | | | TCTCCCTCCTCCAAGCCCTGGACTTCATTTCTCTGTGA |
| 15 | CD90.1 marker, | P | MNPAISVALLLSVLQVSRGQKVTSLTACLVNQNLRLDCRHE |
| | mouse | | NNTKDNSIQHEFSLTREKRKHVLSGTLGIPEHTYRSRVTLS |
| | | | NQPYIKVLTLANFTTKDEGDYFCELRVSGANPMSSNKSISV |
| | | | YRDKLVKCGGISLLVQNTSWMLLLLLSLSLLQALDFISL |
| 16 | LTBR, complete | N | ATGCGCCTGCCCCGGGCCTCCTCTCCCTGCGGCCTGGCCTG |
| | AIR, mouse | | GGGGCCACTCCTGCTGGGACTCAGCGGGCTTCTGGTGGCCT |
| | | | CTCAGCCCCAGCTGGTGCCCCCTTATCGCATAGAAAACCAG |
| | | | ACTTGCTGGGACCAGGACAAGGAATACTACGAGCCCATGCA |
| | | | CGACGTCTGCTGCTCCCGCTGTCCCCCAGGCGAGTTTGTCT |
| | | | TTGCGGTATGCAGCCGCAGCCAAGACACGGTTTGCAAGACT |
| | | | TGCCCCCATAATTCCTATAATGAACACTGGAACCATCTCTC |
| | | | CACCTGCCAGCTGTGCCGCCCCTGTGACATTGTGCTGGGCT |
| | | | TTGAGGAGGTTGCCCCTTGCACCAGCGATCGGAAAGCCGAG |
| | | | TGCCGCTGTCAGCCGGGGATGTCCTGTGTGTATCTGGACAA |
| | | | TGAGTGTGTGCACTGTGAGGAGGAGCGGCTTGTACTCTGCC |
| | | | AGCCTGGCACAGAAGCCGAGGTCACAGATGAAATTATGGAT |
| | | | ACTGACGTCAACTGTGTCCCCTGTAAGCCGGGACACTTCCA |
| | | | GAACACTTCCTCCCCTCGAGCCCGCTGTCAACCCCATACCA |
| | | | GATGTGAGATCCAGGGCCTGGTGGAGGCAGCTCCAGGTACC |
| | | | TCCTACTCGGATACCATCTGTAAAAATCCCCCAGAGCCAGG |
| | | | AGCAATGCTACTGCTGGCCATCCTGCTGTCGCTGGTCCTCT |
| | | | TTCTGCTCTTCACCACTGTCCTGGCCTGTGCCTGGGCGGCC |
| | | | GCGAACAGCAGAAGAAACAGAGGCGGCCAGAGCGACTACAT |
| | | | GAACATGACCCCCAGAAGGCCAGGCCTGACCAGAAAGCCCT |
| | | | ACCAGCCCTACGCCCCTGCCAGAGACTTCGCCGCCTACAGA |
| | | | CCCAGAGCCAAGTTCAGCAGATCCGCCGAGACAGCCGCCAA |
| | | | CCTGCAGGATCCCAACCAGCTGTACAACGAGCTGAACCTGG |
| | | | GCAGACGGGAGGAATACGACGTGCTGGAAAAGAAGAGAGCC |
| | | | AGGGACCCCGAGATGGGCGGCAAGCAGCAGAGAAGAAGAAA |
| | | | CCCTCAGGAAGGCGTCTACAACGCCCTGCAGAAAGACAAGA |
| | | | TGGCCGAGGCCTACAGCGAGATCGGCACCAAGGGCGAGAGA |
| | | | AGAAGGGGCAAGGGCCACGATGGCCTGTACCAGGGCCTGTC |
| | | | CACCGCCACCAAGGACACCTACGACGCCCTGCACATGCAGA |
| | | | CCCTGGCCCCCAGAGCCACGAACTTCTCTCTGTTAAAGCAA |
| | | | GCAGGAGACGTGGAAGAAAACCCCGGTCCTATGAACCCAGC |
| | | | CATCAGCGTCGCTCTCCTGCTCTCAGTCTTGCAGGTGTCCC |
| | | | GAGGGCAGAAGGTGACCAGCCTGACAGCCTGCCTGGTGAAC |
| | | | CAAAACCTTCGCCTGGACTGCCGCCATGAGAATAACACCAA |
| | | | GGATAACTCCATCCAGCATGAGTTCAGCCTGACCCGAGAGA |
| | | | AGAGGAAGCACGTGCTCTCAGGCACCCTTGGGATACCCGAG |
| | | | CACACGTACCGCTCCCGCGTCACCCTCTCCAACCAGCCCTA |
| | | | TATCAAGGTCCTTACCCTAGCCAACTTCACCACCAAGGATG |
| | | | AGGGCGACTACTTTTGTGAGCTTCGCGTCTCGGGCGCGAAT |
| | | | CCCATGAGCTCCAATAAAAGTATCAGTGTGTATAGAGACAA |
| | | | GCTGGTCAAGTGTGGCGGCATAAGCCTGCTGGTTCAGAACA |
| | | | CATCCTGGATGCTGCTGCTGCTGCTTTCCCTCTCCCTCCTC |
| | | | CAAGCCCTGGACTTCATTTCTCTGTGA |
| 17 | LTBR, complete | P | MRLPRASSPCGLAWGPLLLGLSGLLVASQPQLVPPYRIENQ |
| | AIR, mouse | | TCWDQDKEYYEPMHDVCCSRCPPGEFVFAVCSRSQDTVCKT |
| | | | CPHNSYNEHWNHLSTCQLCRPCDIVLGFEEVAPCTSDRKAE |
| | | | CRCQPGMSCVYLDNECVHCEEERLVLCQPGTEAEVTDEIMD |
| | | | TDVNCVPCKPGHFQNTSSPRARCQPHTRCEIQGLVEAAPGT |
| | | | SYSDTICKNPPEPGAMLLLAILLSLVLFLLFTTVLACAWAA |
| | | | ANSRRNRGGQSDYMNMTPRRPGLTRKPYQPYAPARDFAAYR |
| | | | PRAKFSRSAETAANLQDPNQLYNELNLGRREEYDVLEKKRA |
| | | | RDPEMGGKQQRRRNPQEGVYNALQKDKMAEAYSEIGTKGER |
| | | | RRGKGHDGLYQGLSTATKDTYDALHMQTLAPRATNFSLLKQ |
| | | | AGDVEENPGPMNPAISVALLLSVLQVSRGQKVTSLTACLVN |
| | | | QNLRLDCRHENNTKDNSIQHEFSLTREKRKHVLSGTLGIPE |
| | | | HTYRSRVTLSNQPYIKVLTLANFTTKDEGDYFCELRVSGAN |
| | | | PMSSNKSISVYRDKLVKCGGISLLVQNTSWMLLLLLSLSLL |
| | | | QALDFISL |
| 18 | LTBR, | N | ATGCGCCTGCCCCGGGCCTCCTCTCCCTGCGGCCTGGCCTG |
| | extracellular and | | GGGGCCACTCCTGCTGGGACTCAGCGGGCTTCTGGTGGCCT |
| | transmembrane | | CTCAGCCCCAGCTGGTGCCCCCTTATCGCATAGAAAACCAG |
| | domain, mouse | | ACTTGCTGGGACCAGGACAAGGAATACTACGAGCCCATGCA |
| | | | CGACGTCTGCTGCTCCCGCTGTCCCCCAGGCGAGTTTGTCT |
| | | | TTGCGGTATGCAGCCGCAGCCAAGACACGGTTTGCAAGACT |
| | | | TGCCCCCATAATTCCTATAATGAACACTGGAACCATCTCTC |
| | | | CACCTGCCAGCTGTGCCGCCCCTGTGACATTGTGCTGGGCT |
| | | | TTGAGGAGGTTGCCCCTTGCACCAGCGATCGGAAAGCCGAG |
| | | | TGCCGCTGTCAGCCGGGGATGTCCTGTGTGTATCTGGACAA |
| | | | TGAGTGTGTGCACTGTGAGGAGGAGCGGCTTGTACTCTGCC |
| | | | AGCCTGGCACAGAAGCCGAGGTCACAGATGAAATTATGGAT |
| | | | ACTGACGTCAACTGTGTCCCCTGTAAGCCGGGACACTTCCA |
| | | | GAACACTTCCTCCCCTCGAGCCCGCTGTCAACCCCATACCA |
| | | | GATGTGAGATCCAGGGCCTGGTGGAGGCAGCTCCAGGTACC |
| | | | TCCTACTCGGATACCATCTGTAAAAATCCCCCAGAGCCAGG |
| | | | AGCAATGCTACTGCTGGCCATCCTGCTGTCGCTGGTCCTCT |
| | | | TTCTGCTCTTCACCACTGTCCTGGCCTGTGCCTGGGCGGCC |
| | | | GCG |
| 19 | LTBR, | P | MRLPRASSPCGLAWGPLLLGLSGLLVASQPQLVPPYRIENQ |
| | extracellular and | | TCWDQDKEYYEPMHDVCCSRCPPGEFVFAVCSRSQDTVCKT |
| | transmembrane | | CPHNSYNEHWNHLSTCQLCRPCDIVLGFEEVAPCTSDRKAE |
| | domain, mouse | | CRCQPGMSCVYLDNECVHCEEERLVLCQPGTEAEVTDEIMD |
| | | | TDVNCVPCKPGHFQNTSSPRARCQPHTRCEIQGLVEAAPGT |
| | | | SYSDTICKNPPEPGAMLLLAILLSLVLFLLFTTVLACAWAA |
| | | | A |
| 20 | DR3, complete | N | ATGGAGGCACGGCTGCTGCGGGGCTGCGTGGTGGAGCCTCT |
| | AIR, mouse | | GTTCCTACCACTGCTGCTGCTGCTGCTGCTGCTGCTTGGTG |
| | | | GCCAGGGCCAGGGCGGCATGTCTGGCAGGTGTGACTGTGCC |
| | | | AGTGAGTCCCAGAAGAGGTATGGCCCGTTTTGTTGCAGGGG |
| | | | CTGCCCAAAGGGACACTACATGAAGGCCCCCTGCGCAGAAC |
| | | | CCTGTGGCAACTCCACCTGCCTTCCCTGTCCCTCGGACACC |
| | | | TTCTTGACCAGAGACAACCACTTTAAGACTGACTGTACCCG |
| | | | CTGCCAAGTCTGTGATGAAGAGGCCCTTCAAGTGACCCTTG |
| | | | AGAACTGCTCGGCAAAGTCGGACACCCACTGTGGCTGCCAG |
| | | | TCAGGCTGGTGTGTTGACTGCTCCACCGAGCCATGTGGGAA |
| | | | AAGCTCACCTTTCTCTTGTGTCCCATGCGGGGCTACAACAC |
| | | | CAGTCCATGAGGCTCCAACCCCCCGGCCCTGCCTGCCTGGC |
| | | | TTCTATATACGTGGCAATGACTGCACGTCCTGCCCCACGGG |
| | | | CTTCAGCAGCGTTTGCCCTAAGGCTTGCACTGCTGTCIGTG |
| | | | GCTGGAAGCAGATGTTTTGGGTCCAGGTGCTTCTAGGAGTC |
| | | | GCGTTCCTTTTTGGGGCTATCCTGATCTGTGCATATTGTAA |
| | | | CAGCAGAAGAAACAGAGGCGGCCAGAGCGACTACATGAACA |
| | | | TGACCCCCAGAAGGCCAGGCCTGACCAGAAAGCCCTACCAG |
| | | | CCCTACGCCCCTGCCAGAGACTTCGCCGCCTACAGACCCAG |
| | | | AGCCAAGTTCAGCAGATCCGCCGAGACAGCCGCCAACCTGC |
| | | | AGGATCCCAACCAGCTGTACAACGAGCTGAACCTGGGCAGA |
| | | | CGGGAGGAATACGACGTGCTGGAAAAGAAGAGAGCCAGGGA |
| | | | CCCCGAGATGGGCGGCAAGCAGCAGAGAAGAAGAAACCCTC |
| | | | AGGAAGGCGTCTACAACGCCCTGCAGAAAGACAAGATGGCC |
| | | | GAGGCCTACAGCGAGATCGGCACCAAGGGCGAGAGAAGAAG |
| | | | GGGCAAGGGCCACGATGGCCTGTACCAGGGCCTGTCCACCG |
| | | | CCACCAAGGACACCTACGACGCCCTGCACATGCAGACCCTG |
| | | | GCCCCCAGAGCCACGAACTTCTCTCTGTTAAAGCAAGCAGG |
| | | | AGACGTGGAAGAAAACCCCGGTCCTATGAACCCAGCCATCA |
| | | | GCGTCGCTCTCCTGCTCTCAGTCTTGCAGGTGTCCCGAGGG |
| | | | CAGAAGGTGACCAGCCTGACAGCCTGCCTGGTGAACCAAAA |
| | | | CCTTCGCCTGGACTGCCGCCATGAGAATAACACCAAGGATA |
| | | | ACTCCATCCAGCATGAGTTCAGCCTGACCCGAGAGAAGAGG |
| | | | AAGCACGTGCTCTCAGGCACCCTTGGGATACCCGAGCACAC |
| | | | GTACCGCTCCCGCGTCACCCTCTCCAACCAGCCCTATATCA |
| | | | AGGTCCTTACCCTAGCCAACTTCACCACCAAGGATGAGGGC |
| | | | GACTACTTTTGTGAGCTTCGCGTCTCGGGCGCGAATCCCAT |
| | | | GAGCTCCAATAAAAGTATCAGTGTGTATAGAGACAAGCTGG |
| | | | TCAAGTGTGGCGGCATAAGCCTGCTGGTTCAGAACACATCC |
| | | | TGGATGCTGCTGCTGCTGCTTTCCCTCTCCCTCCTCCAAGC |
| | | | CCTGGACTTCATTTCTCTGTGA |
| 21 | DR3, complete | P | MEARLLRGCVVEPLFLPLLLLLLLLLGGQGQGGMSGRCDCA |
| | AIR, mouse | | SESQKRYGPFCCRGCPKGHYMKAPCAEPCGNSTCLPCPSDT |
| | | | FLTRDNHFKTDCTRCQVCDEEALQVTLENCSAKSDTHCGCQ |
| | | | SGWCVDCSTEPCGKSSPFSCVPCGATTPVHEAPTPRPCLPG |
| | | | FYIRGNDCTSCPTGFSSVCPKACTAVCGWKQMEWVQVLLGV |
| | | | AFLFGAILICAYCNSRRNRGGQSDYMNMTPRRPGLTRKPYQ |
| | | | PYAPARDFAAYRPRAKFSRSAETAANLQDPNQLYNELNLGR |
| | | | REEYDVLEKKRARDPEMGGKQQRRRNPQEGVYNALQKDKMA |
| | | | EAYSEIGTKGERRRGKGHDGLYQGLSTATKDTYDALHMQTL |
| | | | APRATNFSLLKQAGDVEENPGPMNPAISVALLLSVLQVSRG |
| | | | QKVTSLTACLVNQNLRLDCRHENNTKDNSIQHEFSLTREKR |
| | | | KHVLSGTLGIPEHTYRSRVTLSNQPYIKVLTLANFTTKDEG |
| | | | DYFCELRVSGANPMSSNKSISVYRDKLVKCGGISLLVQNTS |
| | | | WMLLLLLSLSLLQALDFISL |
| 22 | DR3, extracellular | N | ATGGAGGCACGGCTGCTGCGGGGCTGCGTGGTGGAGCCTCT |
| | and | | GTTCCTACCACTGCTGCTGCTGCTGCTGCTGCTGCTTGGTG |
| | transmembrane | | GCCAGGGCCAGGGCGGCATGTCTGGCAGGTGTGACTGTGCC |
| | domain, mouse | | AGTGAGTCCCAGAAGAGGTATGGCCCGTTTTGTTGCAGGGG |
| | | | CTGCCCAAAGGGACACTACATGAAGGCCCCCTGCGCAGAAC |
| | | | CCTGTGGCAACTCCACCTGCCTTCCCTGTCCCTCGGACACC |
| | | | TTCTTGACCAGAGACAACCACTTTAAGACTGACTGTACCCG |
| | | | CTGCCAAGTCTGTGATGAAGAGGCCCTTCAAGTGACCCTTG |
| | | | AGAACTGCTCGGCAAAGTCGGACACCCACTGTGGCTGCCAG |
| | | | TCAGGCTGGTGTGTTGACTGCTCCACCGAGCCATGTGGGAA |
| | | | AAGCTCACCTTTCTCTTGTGTCCCATGCGGGGCTACAACAC |
| | | | CAGTCCATGAGGCTCCAACCCCCCGGCCCTGCCTGCCTGGC |
| | | | TTCTATATACGTGGCAATGACTGCACGTCCTGCCCCACGGG |
| | | | CTTCAGCAGCGTTTGCCCTAAGGCTTGCACTGCTGTCTGTG |
| | | | GCTGGAAGCAGATGTTTTGGGTCCAGGTGCTTCTAGGAGTC |
| | | | GCGTTCCTTTTTGGGGCTATCCTGATCTGTGCATATTGT |
| 23 | DR3, extracellular | P | MEARLLRGCVVEPLFLPLLLLLLLLLGGQGQGGMSGRCDCA |
| | and | | SESQKRYGPFCCRGCPKGHYMKAPCAEPCGNSTCLPCPSDT |
| | transmembrane | | FLTRDNHFKTDCTRCQVCDEEALQVTLENCSAKSDTHCGCQ |
| | domain, mouse | | SGWCVDCSTEPCGKSSPFSCVPCGATTPVHEAPTPRPCLPG |
| | | | FYIRGNDCTSCPTGFSSVCPKACTAVCGWKQMEWVQVLLGV |
| | | | AFLFGAILICAYC |
| 24 | Truncated control | N | ATGGCGCCCGCCGCCCTCTGGGTCGCGCTGGTCTTCGAACT |
| | AIR, complete | | GCAGCTGTGGGCCACCGGGCACACATTCTGGGCCCTGGTGG |
| | | | TGGTGGCCGGCGTGCTGTTCTGTTACGGCCTGCTGGTCACA |
| | | | GTGGCCCTGTGCGTGATCTGGACCAACAGCAGAAGAAACAG |
| | | | AGGCGGCCAGAGCGACTACATGAACATGACCCCCAGAAGGC |
| | | | CAGGCCTGACCAGAAAGCCCTACCAGCCCTACGCCCCTGCC |
| | | | AGAGACTTCGCCGCCTACAGACCCAGAGCCAAGTTCAGCAG |
| | | | ATCCGCCGAGACAGCCGCCAACCTGCAGGATCCCAACCAGC |
| | | | TGTACAACGAGCTGAACCTGGGCAGACGGGAGGAATACGAC |
| | | | GTGCTGGAAAAGAAGAGAGCCAGGGACCCCGAGATGGGCGG |
| | | | CAAGCAGCAGAGAAGAAGAAACCCTCAGGAAGGCGTCTACA |
| | | | ACGCCCTGCAGAAAGACAAGATGGCCGAGGCCTACAGCGAG |
| | | | ATCGGCACCAAGGGCGAGAGAAGAAGGGGCAAGGGCCACGA |
| | | | TGGCCTGTACCAGGGCCTGTCCACCGCCACCAAGGACACCT |
| | | | ACGACGCCCTGCACATGCAGACCCTGGCCCCCAGAGCCACG |
| | | | AACTTCTCTCTGTTAAAGCAAGCAGGAGACGTGGAAGAAAA |
| | | | CCCCGGTCCTATGAACCCAGCCATCAGCGTCGCTCTCCTGC |
| | | | TCTCAGTCTTGCAGGTGTCCCGAGGGCAGAAGGTGACCAGC |
| | | | CTGACAGCCTGCCTGGTGAACCAAAACCTTCGCCTGGACTG |
| | | | CCGCCATGAGAATAACACCAAGGATAACTCCATCCAGCATG |
| | | | AGTTCAGCCTGACCCGAGAGAAGAGGAAGCACGTGCTCTCA |
| | | | GGCACCCTTGGGATACCCGAGCACACGTACCGCTCCCGCGT |
| | | | CACCCTCTCCAACCAGCCCTATATCAAGGTCCTTACCCTAG |
| | | | CCAACTTCACCACCAAGGATGAGGGCGACTACTTTTGTGAG |
| | | | CTTCGCGTCTCGGGCGCGAATCCCATGAGCTCCAATAAAAG |
| | | | TATCAGTGTGTATAGAGACAAGCTGGTCAAGTGTGGCGGCA |
| | | | TAAGCCTGCTGGTTCAGAACACATCCTGGATGCTGCTGCTG |
| | | | CTGCTTTCCCTCTCCCTCCTCCAAGCCCTGGACTTCATTTC |
| | | | TCTGTGA |
| 25 | Truncated control | P | MAPAALWVALVFELQLWATGHTEWALVVVAGVLFCYGLLVT |
| | AIR, complete | | VALCVIWTNSRRNRGGQSDYMNMT PRRPGLTRKPYQPYAPA |
| | | | RDFAAYRPRAKFSRSAETAANLQDPNQLYNELNLGRREEYD |
| | | | VLEKKRARDPEMGGKQQRRRNPQEGVYNALQKDKMAEAYSE |
| | | | IGTKGERRRGKGHDGLYQGLSTATKDTYDALHMQTLAPRAT |
| | | | NFSLLKQAGDVEENPGPMNPAISVALLLSVLQVSRGQKVTS |
| | | | LTACLVNQNLRLDCRHENNTKDNSIQHEFSLTREKRKHVLS |
| | | | GTLGIPEHTYRSRVTLSNQPYIKVLTLANFTTKDEGDYFCE |
| | | | LRVSGANPMSSNKSISVYRDKLVKCGGISLLVQNTSWMLLL |
| | | | LLSLSLLQALDFISL |
| 26 | TNFR2 signaling | N | ATGGCGCCCGCCGCCCTCTGGGTCGCGCTGGTCTTCGAACT |
| | peptide, mouse | | GCAGCTGTGGGCCACCGGGCACACA |
| 27 | TNFR2 signaling | P | MAPAALWVALVFELQLWATGHT |
| | peptide, mouse | | |
| 28 | CD28, | N | TTCTGGGCCCTGGTGGTGGTGGCCGGCGTGCTGTTCTGTTA |
| | transmembrane | | CGGCCTGCTGGTCACAGTGGCCCTGTGCGTGATCTGGACC |
| | domain, mouse | | |
| 29 | CD28, | P | FWALVVVAGVLFCYGLLVTVALCVIWT |
| | transmembrane | | |
| | domain, mouse | | |
| 30 | LTBR, complete | N | ATGCTCCTGCCTTGGGCCACCTCTGCCCCCGGCCTGGCCTG |
| | AIR, human | | GGGGCCTCTGGTGCTGGGCCTCTTCGGGCTCCTGGCAGCAT |
| | | | CGCAGCCCCAGGCGGTGCCTCCATATGCGTCGGAGAACCAG |
| | | | ACCTGCAGGGACCAGGAAAAGGAATACTATGAGCCCCAGCA |
| | | | CCGCATCTGCTGCTCCCGCTGCCCGCCAGGCACCTATGTCT |
| | | | CAGCTAAATGTAGCCGCATCCGGGACACAGTTTGTGCCACA |
| | | | TGTGCCGAGAATTCCTACAACGAGCACTGGAACTACCTGAC |
| | | | CATCTGCCAGCTGTGCCGCCCCTGTGACCCAGTGATGGGCC |
| | | | TCGAGGAGATTGCCCCCTGCACAAGCAAACGGAAGACCCAG |
| | | | TGCCGCTGCCAGCCGGGAATGTTCTGTGCTGCCTGGGCCCT |
| | | | CGAGTGTACACACTGCGAGCTACTTTCTGACTGCCCGCCTG |
| | | | GCACTGAAGCCGAGCTCAAAGATGAAGTTGGGAAGGGTAAC |
| | | | AACCACTGCGTCCCCTGCAAGGCCGGGCACTTCCAGAATAC |
| | | | CTCCTCCCCCAGCGCCCGCTGCCAGCCCCACACCAGGTGTG |
| | | | AGAACCAAGGTCTGGTGGAGGCAGCTCCAGGCACTGCCCAG |
| | | | TCCGACACAACCTGCAAAAATCCATTAGAGCCACTGCCCCC |
| | | | AGAGATGTCAGGAACCATGCTGATGCTGGCCGTTCTGCTGC |
| | | | CACTGGCCTTCTTTCTGCTCCTTGCCACCGTCTTCTCCTGC |
| | | | ATCTGGAGGAGTAAGAGGAGCAGGCTCCTGCACAGTGACTA |
| | | | CATGAACATGACTCCCCGCCGCCCCGGGCCCACCCGCAAGC |
| | | | ATTACCAGCCCTATGCCCCCCCACGCGACTTCGCAGCCTAT |
| | | | CGCTCCCTGAGAGTGAAGTTCAGCAGGAGCGCAGACGCCCC |
| | | | CGCGTACCAGCAGGGCCAGAACCAGCTCTATAACGAGCTCA |
| | | | ATCTAGGACGAAGAGAGGAGTACGATGTTTTGGACAAGAGA |
| | | | CGTGGCCGGGACCCTGAGATGGGGGGAAAGCCGAGAAGGAA |
| | | | GAACCCTCAGGAAGGCCTGTACAATGAACTGCAGAAAGATA |
| | | | AGATGGCGGAGGCCTACAGTGAGATTGGGATGAAAGGCGAG |
| | | | CGCCGGAGGGGCAAGGGGCACGATGGCCTTTACCAGGGTCT |
| | | | CAGTACAGCCACCAAGGACACCTACGACGCCCTTCACATGC |
| | | | AGGCCCTGCCCCCTCGCGCCACGAACTTCTCTCTGTTAAAG |
| | | | CAAGCAGGAGACGTGGAAGAAAACCCCGGTCCTATGAACCC |
| | | | AGCCATCAGCGTCGCTCTCCTGCTCTCAGTCTTGCAGGTGT |
| | | | CCCGAGGGCAGAAGGTGACCAGCCTGACAGCCTGCCTGGTG |
| | | | AACCAAAACCTTCGCCTGGACTGCCGCCATGAGAATAACAC |
| | | | CAAGGATAACTCCATCCAGCATGAGTTCAGCCTGACCCGAG |
| | | | AGAAGAGGAAGCACGTGCTCTCAGGCACCCTTGGGATACCC |
| | | | GAGCACACGTACCGCTCCCGCGTCACCCTCTCCAACCAGCC |
| | | | CTATATCAAGGTCCTTACCCTAGCCAACTTCACCACCAAGG |
| | | | ATGAGGGCGACTACTTTTGTGAGCTTCGCGTCTCGGGCGCG |
| | | | AATCCCATGAGCTCCAATAAAAGTATCAGTGTGTATAGAGA |
| | | | CAAGCTGGTCAAGTGTGGCGGCATAAGCCTGCTGGTTCAGA |
| | | | ACACATCCTGGATGCTGCTGCTGCTGCTTTCCCTCTCCCTC |
| | | | CTCCAAGCCCTGGACTTCATTTCTCTGTGA |
| 31 | LTBR, complete | P | MLLPWATSAPGLAWGPLVLGLFGLLAASQPQAVPPYASENQ |
| | AIR, human | | TCRDQEKEYYEPQHRICCSRCPPGTYVSAKCSRIRDTVCAT |
| | | | CAENSYNEHWNYLTICQLCRPCDPVMGLEEIAPCTSKRKTQ |
| | | | CRCQPGMFCAAWALECTHCELLSDCPPGTEAELKDEVGKGN |
| | | | NHCVPCKAGHFQNTSSPSARCQPHTRCENQGLVEAAPGTAQ |
| | | | SDTTCKNPLEPLPPEMSGTMLMLAVLLPLAFFLLLATVFSC |
| | | | IWRSKRSRLLHSDYMNMTPRRPGPTRKHYQPYAPPRDFAAY |
| | | | RSLRVKFSRSADAPAYQQGQNQLYNELNLGRREEYDVLDKR |
| | | | RGRDPEMGGKPRRKNPQEGLYNELQKDKMAEAYSEIGMKGE |
| | | | RRRGKGHDGLYQGLSTATKDTYDALHMQALPPRATNFSLLK |
| | | | QAGDVEENPGPMNPAISVALLLSVLQVSRGQKVTSLTACLV |
| | | | NQNLRLDCRHENNTKDNSIQHEFSLTREKRKHVLSGTLGIP |
| | | | EHTYRSRVTLSNQPYIKVLTLANFTTKDEGDYFCELRVSGA |
| | | | NPMSSNKSISVYRDKLVKCGGISLLVQNTSWMLLLLLSLSL |
| | | | LQALDFISL |
| 32 | LTBR, | N | ATGCTCCTGCCTTGGGCCACCTCTGCCCCCGGCCTGGCCTG |
| | extracellular and | | GGGGCCTCTGGTGCTGGGCCTCTTCGGGCTCCTGGCAGCAT |
| | transmembrane | | CGCAGCCCCAGGCGGTGCCTCCATATGCGTCGGAGAACCAG |
| | domain, human | | ACCTGCAGGGACCAGGAAAAGGAATACTATGAGCCCCAGCA |
| | | | CCGCATCTGCTGCTCCCGCTGCCCGCCAGGCACCTATGTCT |
| | | | CAGCTAAATGTAGCCGCATCCGGGACACAGTTTGTGCCACA |
| | | | TGTGCCGAGAATTCCTACAACGAGCACTGGAACTACCTGAC |
| | | | CATCTGCCAGCTGTGCCGCCCCTGTGACCCAGTGATGGGCC |
| | | | TCGAGGAGATTGCCCCCTGCACAAGCAAACGGAAGACCCAG |
| | | | TGCCGCTGCCAGCCGGGAATGTTCTGTGCTGCCTGGGCCCT |
| | | | CGAGTGTACACACTGCGAGCTACTTTCTGACTGCCCGCCTG |
| | | | GCACTGAAGCCGAGCTCAAAGATGAAGTTGGGAAGGGTAAC |
| | | | AACCACTGCGTCCCCTGCAAGGCCGGGCACTTCCAGAATAC |
| | | | CTCCTCCCCCAGCGCCCGCTGCCAGCCCCACACCAGGTGTG |
| | | | AGAACCAAGGTCTGGTGGAGGCAGCTCCAGGCACTGCCCAG |
| | | | TCCGACACAACCTGCAAAAATCCATTAGAGCCACTGCCCCC |
| | | | AGAGATGTCAGGAACCATGCTGATGCTGGCCGTTCTGCTGC |
| | | | CACTGGCCTTCTTTCTGCTCCTTGCCACCGTCTTCTCCTGC |
| | | | ATCTGG |
| 33 | LTBR, | P | MLLPWATSAPGLAWGPLVLGLFGLLAASQPQAVPPYASENQ |
| | extracellular and | | TCRDQEKEYYEPQHRICCSRCPPGTYVSAKCSRIRDTVCAT |
| | transmembrane | | CAENSYNEHWNYLTICQLCRPCDPVMGLEEIAPCTSKRKTQ |
| | domain, human | | CRCQPGMFCAAWALECTHCELLSDCPPGTEAELKDEVGKGN |
| | | | NHCVPCKAGHFQNTSSPSARCQPHTRCENQGLVEAAPGTAQ |
| | | | SDTTCKNPLEPLPPEMSGTMLMLAVLLPLAFFLLLATVFSC |
| | | | IW |
| 34 | CD28 signaling | N | AGGAGTAAGAGGAGCAGGCTCCTGCACAGTGACTACATGAA |
| | domain, human | | CATGACTCCCCGCCGCCCCGGGCCCACCCGCAAGCATTACC |
| | | | AGCCCTATGCCCCCCCACGCGACTTCGCAGCCTATCGCTCC |
| 35 | CD28 signaling | P | RSKRSRLLHSDYMNMTPRRPGPTRKHYQPYAPPRDFAAYRS |
| | domain, human | | |
| 36 | CD3 zeta domain, | N | CTGAGAGTGAAGTTCAGCAGGAGCGCAGACGCCCCCGCGTA |
| | human | | CCAGCAGGGCCAGAACCAGCTCTATAACGAGCTCAATCTAG |
| | | | GACGAAGAGAGGAGTACGATGTTTTGGACAAGAGACGTGGC |
| | | | CGGGACCCTGAGATGGGGGGAAAGCCGAGAAGGAAGAACCC |
| | | | TCAGGAAGGCCTGTACAATGAACTGCAGAAAGATAAGATGG |
| | | | CGGAGGCCTACAGTGAGATTGGGATGAAAGGCGAGCGCCGG |
| | | | AGGGGCAAGGGGCACGATGGCCTTTACCAGGGTCTCAGTAC |
| | | | AGCCACCAAGGACACCTACGACGCCCTTCACATGCAGGCCC |
| | | | TGCCCCCTCGC |
| 37 | CD3 zeta domain, | P | LRVKFSRSADAPAYQQGQNQLYNELNLGRREEYDVLDKRRG |
| | human | | RDPEMGGKPRRKNPQEGLYNELQKDKMAEAYSEIGMKGERR |
| | | | RGKGHDGLYQGLSTATKDTYDALHMQALPPR |
| 38 | TNFR2, complete | P | MAPVAVWAALAVGLELWAAAHALPAQVAFTPYAPEPGSTCR |
| | AIR, human | | LREYYDQTAQMCCSKCSPGQHAKVFCTKTSDTVCDSCEDST |
| | | | YTQLWNWVPECLSCGSRCSSDQVETQACTREQNRICTCRPG |
| | | | WYCALSKQEGCRLCAPLRKCRPGFGVARPGTETSDVVCKPC |
| | | | APGTFSNTTSSTDICRPHQICNVVAIPGNASMDAVCTSTSP |
| | | | TRSMAPGAVHLPQPVSTRSQHTQPTPEPSTAPSTSFLLPMG |
| | | | PSPPAEGSTGDFALPVGLIVGVTALGLLIIGVVNCVIMTQV |
| | | | RSKRSRLLHSDYMNMTPRRPGPTRKHYQPYAPPRDFAAYRS |
| | | | LRVKFSRSADAPAYQQGQNQLYNELNLGRREEYDVLDKRRG |
| | | | RDPEMGGKPRRKNPQEGLYNELQKDKMAEAYSEIGMKGERR |
| | | | RGKGHDGLYQGLSTATKDTYDALHMQALPPR |
| 39 | TNFR2, | P | MAPVAVWAALAVGLELWAAAHALPAQVAFTPYAPEPGSTCR |
| | extracellular and | | LREYYDQTAQMCCSKCSPGQHAKVFCTKTSDTVCDSCEDST |
| | transmembrane | | YTQLWNWVPECLSCGSRCSSDQVETQACTREQNRICTCRPG |
| | domain, human | | WYCALSKQEGCRLCAPLRKCRPGFGVARPGTETSDVVCKPC |
| | | | APGTFSNTTSSTDICRPHQICNVVAIPGNASMDAVCTSTSP |
| | | | TRSMAPGAVHLPQPVSTRSQHTQPTPEPSTAPSTSFLLPMG |
| | | | PSPPAEGSTGDFALPVGLIVGVTALGLLIIGVVNCVIMTQV |
| 40 | DR3, complete | P | MEQRPRGCAAVAAALLLVLLGARAQGGTRSPRCDCAGDFHK |
| | AIR, human | | KIGLFCCRGCPAGHYLKAPCTEPCGNSTCLVCPQDTFLAWE |
| | | | NHHNSECARCQACDEQASQVALENCSAVADTRCGCKPGWFV |
| | | | ECQVSQCVSSSPFYCQPCLDCGALHRHTRLLCSRRDTDCGT |
| | | | CLPGFYEHGDGCVSCPTSTLGSCPERCAAVCGWRQMFWVQV |
| | | | LLAGLVVPLLLGATLRSKRSRLLHSDYMNMTPRRPGPTRKH |
| | | | YQPYAPPRDFAAYRSLRVKFSRSADAPAYQQGQNQLYNELN |
| | | | LGRREEYDVLDKRRGRDPEMGGKPRRKNPQEGLYNELQKDK |
| | | | MAEAYSEIGMKGERRRGKGHDGLYQGLSTATKDTYDALHMQ |
| | | | ALPPR |
| 41 | DR3, extracellular | P | MEQRPRGCAAVAAALLLVLLGARAQGGTRSPRCDCAGDFHK |
| | and | | KIGLFCCRGCPAGHYLKAPCTEPCGNSTCLVCPQDTFLAWE |
| | transmembrane | | NHHNSECARCQACDEQASQVALENCSAVADTRCGCKPGWFV |
| | domain, human | | ECQVSQCVSSSPFYCQPCLDCGALHRHTRLLCSRRDTDCGT |
| | | | CLPGFYEHGDGCVSCPTSTLGSCPERCAAVCGWRQMFWVQV |
| | | | LLAGLVVPLLLGATL |
| 42 | CD40, complete | N | ATGGTGTCTTTGCCTCGGCTGTGCGCGCTATGGGGCTGCTT |
| | AIR, mouse | | GTTGACAGCGGTCCATCTAGGGCAGTGTGTTACGTGCAGTG |
| | | | ACAAACAGTACCTCCACGATGGCCAGTGCTGTGATTTGTGC |
| | | | CAGCCAGGAAGCCGACTGACAAGCCACTGCACAGCTCTTGA |
| | | | GAAGACCCAATGCCACCCATGTGACTCAGGCGAATTCTCAG |
| | | | CCCAGTGGAACAGGGAGATTCGCTGTCACCAGCACAGACAC |
| | | | TGTGAACCCAATCAAGGGCTTCGGGTTAAGAAGGAGGGCAC |
| | | | CGCAGAATCAGACACTGTCTGTACCTGTAAGGAAGGACAAC |
| | | | ACTGCACCAGCAAGGATTGCGAGGCATGTGCTCAGCACACG |
| | | | CCCTGTATCCCTGGCTTTGGAGTTATGGAGATGGCCACTGA |
| | | | GACCACTGATACCGTCTGTCATCCCTGCCCAGTCGGCTTCT |
| | | | TCTCCAATCAGTCATCACTTTTCGAAAAGTGTTATCCCTGG |
| | | | ACAAGCTGTGAGGATAAGAACTTGGAGGTCCTACAGAAAGG |
| | | | AACGAGTCAGACTAATGTCATCTGTGGTTTAAAGTCCCGGA |
| | | | TGCGAGCCCTGCTGGTCATTCCTGTCGTGATGGGCATCCTC |
| | | | ATCACCATTTTCGGGGTGTTTCTCTATATCAACAGCAGAAG |
| | | | AAACAGAGGCGGCCAGAGCGACTACATGAACATGACCCCCA |
| | | | GAAGGCCAGGCCTGACCAGAAAGCCCTACCAGCCCTACGCC |
| | | | CCTGCCAGAGACTTCGCCGCCTACAGACCCAGAGCCAAGTT |
| | | | CAGCAGATCCGCCGAGACAGCCGCCAACCTGCAGGATCCCA |
| | | | ACCAGCTGTACAACGAGCTGAACCTGGGCAGACGGGAGGAA |
| | | | TACGACGTGCTGGAAAAGAAGAGAGCCAGGGACCCCGAGAT |
| | | | GGGCGGCAAGCAGCAGAGAAGAAGAAACCCTCAGGAAGGCG |
| | | | TCTACAACGCCCTGCAGAAAGACAAGATGGCCGAGGCCTAC |
| | | | AGCGAGATCGGCACCAAGGGCGAGAGAAGAAGGGGCAAGGG |
| | | | CCACGATGGCCTGTACCAGGGCCTGTCCACCGCCACCAAGG |
| | | | ACACCTACGACGCCCTGCACATGCAGACCCTGGCCCCCAGA |
| 43 | CD40, complete | P | MVSLPRLCALWGCLLTAVHLGQCVTCSDKQYLHDGQCCDLC |
| | AIR with CD28 | | QPGSRLTSHCTALEKTQCHPCDSGEFSAQWNREIRCHQHRH |
| | costim, mouse | | CEPNQGLRVKKEGTAESDTVCTCKEGQHCTSKDCEACAQHT |
| | | | PCIPGFGVMEMATETTDTVCHPCPVGFFSNQSSLFEKCYPW |
| | | | TSCEDKNLEVLQKGTSQTNVICGLKSRMRALLVIPVVMGIL |
| | | | ITIFGVFLYINSRRNRGGQSDYMNMTPRRPGLTRKPYQPYA |
| | | | PARDFAAYRPRAKFSRSAETAANLQDPNQLYNELNLGRREE |
| | | | YDVLEKKRARDPEMGGKQQRRRNPQEGVYNALQKDKMAEAY |
| | | | SEIGTKGERRRGKGHDGLYQGLSTATKDTYDALHMQTLAPR |
| 44 | CD40, | N | ATGGTGTCTTTGCCTCGGCTGTGCGCGCTATGGGGCTGCTT |
| | extracellular and | | GTTGACAGCGGTCCATCTAGGGCAGTGTGTTACGTGCAGTG |
| | transmembrane | | ACAAACAGTACCTCCACGATGGCCAGTGCTGTGATTTGTGC |
| | domain, mouse | | CAGCCAGGAAGCCGACTGACAAGCCACTGCACAGCTCTTGA |
| | | | GAAGACCCAATGCCACCCATGTGACTCAGGCGAATTCTCAG |
| | | | CCCAGTGGAACAGGGAGATTCGCTGTCACCAGCACAGACAC |
| | | | TGTGAACCCAATCAAGGGCTTCGGGTTAAGAAGGAGGGCAC |
| | | | CGCAGAATCAGACACTGTCTGTACCTGTAAGGAAGGACAAC |
| | | | ACTGCACCAGCAAGGATTGCGAGGCATGTGCTCAGCACACG |
| | | | CCCTGTATCCCTGGCTTTGGAGTTATGGAGATGGCCACTGA |
| | | | GACCACTGATACCGTCTGTCATCCCTGCCCAGTCGGCTTCT |
| | | | TCTCCAATCAGTCATCACTTTTCGAAAAGTGTTATCCCTGG |
| | | | ACAAGCTGTGAGGATAAGAACTTGGAGGTCCTACAGAAAGG |
| | | | AACGAGTCAGACTAATGTCATCTGTGGTTTAAAGTCCCGGA |
| | | | TGCGAGCCCTGCTGGTCATTCCTGTCGTGATGGGCATCCTC |
| | | | ATCACCATTTTCGGGGTGTTTCTCTATATC |
| 45 | CD40, | P | MVSLPRLCALWGCLLTAVHLGQCVTCSDKQYLHDGQCCDLC |
| | extracellular and | | QPGSRLTSHCTALEKTQCHPCDSGEFSAQWNREIRCHQHRH |
| | transmembrane | | CEPNQGLRVKKEGTAESDTVCTCKEGQHCTSKDCEACAQHT |
| | domain, mouse | | PCIPGFGVMEMATETTDTVCHPCPVGFFSNQSSLFEKCYPW |
| | | | TSCEDKNLEVLQKGTSQTNVICGLKSRMRALLVIPVVMGIL |
| | | | ITIFGVFLYI |
| 46 | CD40, | P | MVRLPLQCVLWGCLLTAVHPEPPTACREKQYLINSQCCSLC |
| | extracellular and | | QPGQKLVSDCTEFTETECLPCGESEFLDTWNRETHCHQHKY |
| | transmembrane | | CDPNLGLRVQQKGTSETDTICTCEEGWHCTSEACESCVLHR |
| | domain, human | | SCSPGFGVKQIATGVSDTICEPCPVGFFSNVSSAFEKCHPW |
| | | | TSCETKDLVVQQAGTNKTDVVCGPQDRLRALVVIPIIFGIL |
| | | | FAILLVLVFI |
| 47 | CD40, complete | P | MVRLPLQCVLWGCLLTAVHPEPPTACREKQYLINSQCCSLC |
| | AIR with CD28 | | QPGQKLVSDCTEFTETECLPCGESEFLDTWNRETHCHQHKY |
| | costim, human | | CDPNLGLRVQQKGTSETDTICTCEEGWHCTSEACESCVLHR |
| | | | SCSPGFGVKQIATGVSDTICEPCPVGFFSNVSSAFEKCHPW |
| | | | TSCETKDLVVQQAGTNKTDVVCGPQDRLRALVVIPIIFGIL |
| | | | FAILLVLVFIRSKRSRLLHSDYMNMTPRRPGPTRKHYQPYA |
| | | | PPRDFAAYRSLRVKFSRSADAPAYQQGQNQLYNELNLGRRE |
| | | | EYDVLDKRRGRDPEMGGKPRRKNPQEGLYNELQKDKMAEAY |
| | | | SEIGMKGERRRGKGHDGLYQGLSTATKDTYDALHMQALPPR |

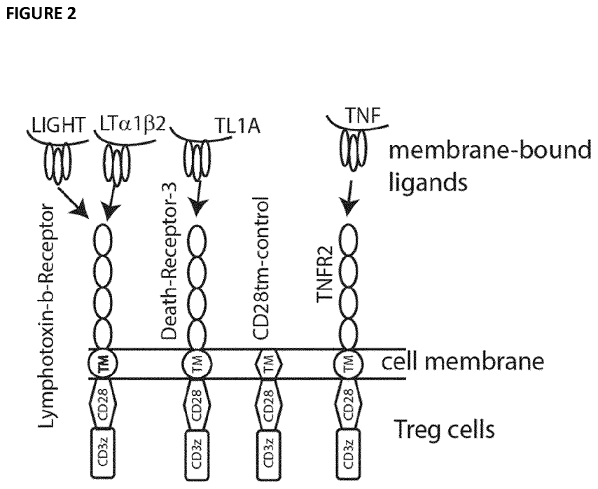

The mode of action of the AIRs is shown in FIG. 2. Binding of membrane bound TNF family members via AIRs leads to translation of the cytokine signal into a T cell receptor (TCR)-like signal. This initiates T cell activation independent of its endogenous TCR-MHC restriction in an environment/inflammation dependent manner.

### Example 5: Transduction and Surface Expression of the AIRs

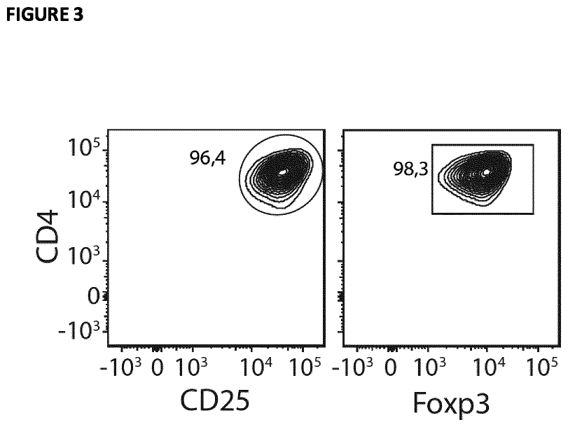

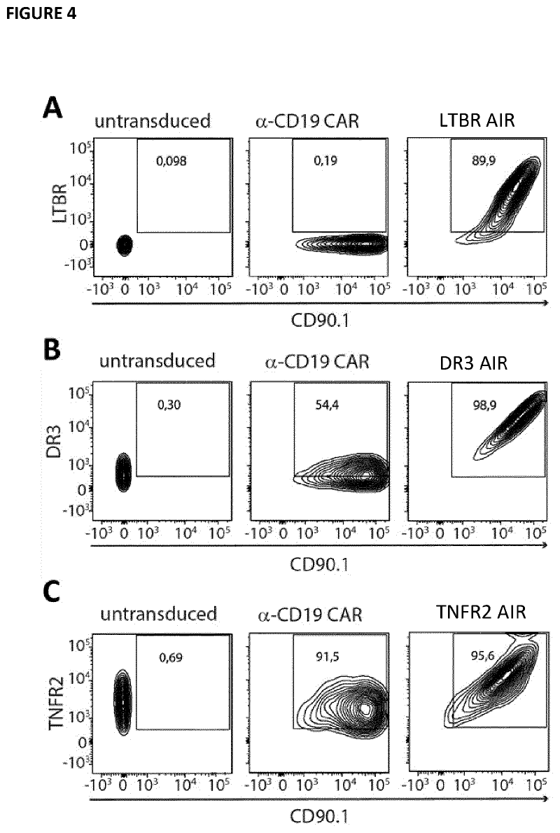

Transduction and expression of the AIRs according to the present disclosure was investigated in murine regulatory T cells (Treg). Tregs were isolated from spleen and lymph nodes of C57/BL6 mice, sorted (naïve CD4+CD25+CD62L+ Treg or Foxp3+ reporter (hCD2+) positive) and expanded via anti-CD3 and anti-CD28 antibody coupled beads plus IL-2 (2000 U/ml) stimulation for 6 days. Purity of Treg cultures was determined with flow cytometry. Treg cultures contained about 90-99% Foxp3+ T cells. On day two after isolation Treg were transduced with AIR constructs or an anti-CD19 Chimeric Antigen Receptor (anti-CD19 CAR) as control. Three days later transduction efficacy via CD90.1 staining and surface expression of AIRs was checked with flow cytometry. Results are shown in FIGS. 3 and 4. Similar results were obtained with a CD40 AIR (93.6% Foxp3+ T cells). It could be demonstrated that all AIRs are efficiently expressed on the surface of regulatory T cells.

### Example 6: AIRs Induce the Expression of T-Cell Receptor Signaling Marker Genes

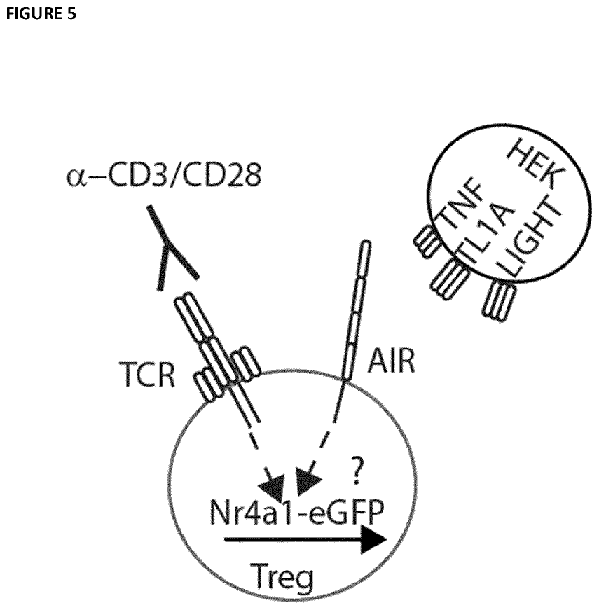

To test if the AIRs of the present disclosure induce the T-cell receptor signaling, the induction of Nr4a1 was investigated. Nr4a1 is a well-known target gene of T-cell receptor signaling. Treg from Nr4a1-eGFP reporter mice were used. As a control, Treg were stimulated with anti-CD3/CD28 antibody-coupled beads. The outline of the experiment is shown in FIG. 5.

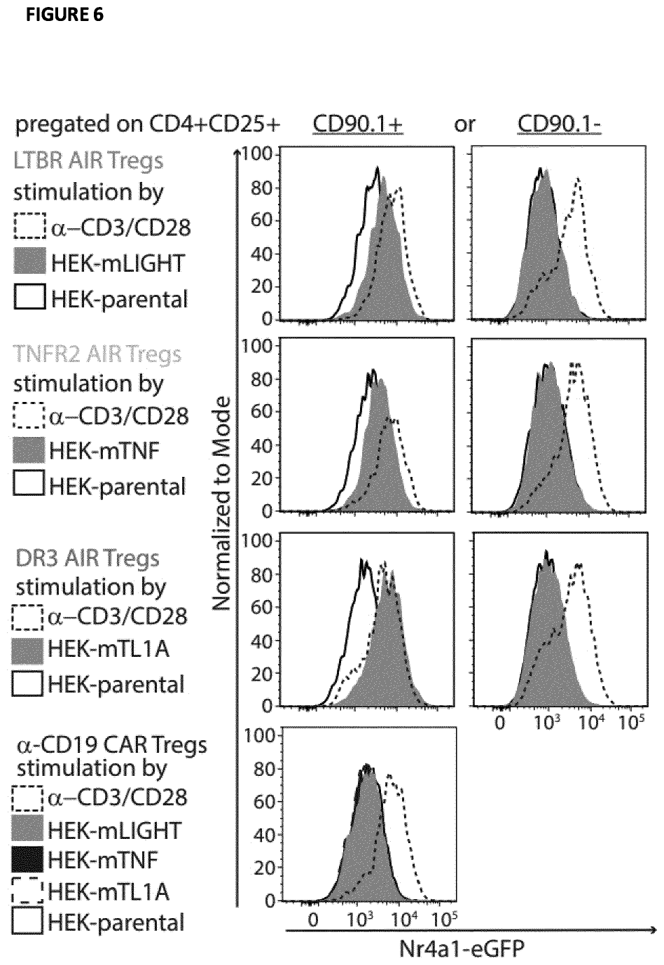

Tregs expressing AIRs were stimulated in over-night co-culture with HEK cells expressing murine Light (mLight), mTL1A or mTNF. As a positive control, Tregs were stimulated with anti-CD3/CD28 antibody-coupled beads to mimic TCR signaling. Results are shown in FIG. 6. Only AIR positive (CD90.1+) Tregs express Nr4a1-eGFP (left), but not AIR negative (CD90.1−) Tregs (right). Anti-CD19 CAR expressing Tregs, in contrast, did not show upregulation of Nr4a1-GFP (lower panel).

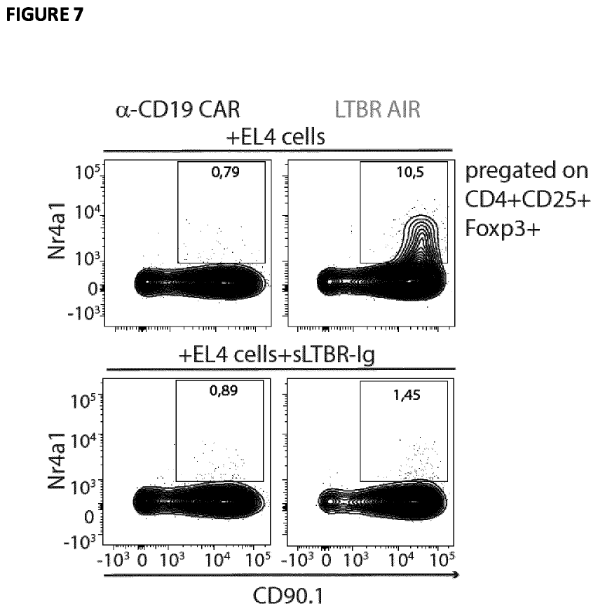

FIG. 7 demonstrates the expression of Nr4a1 upon administration of an AIR according to the present disclosure, exemplified with an AIR carrying carrying the extracellular domain of LTBR. Tregs expressing an LTBR AIR according to the present disclosure or an irrelevant CAR carrying extracellularly an anti-CD19 single chain antibody (scFv) were co-cultured with murine EL4 cells, which are known to express LIGHT, in absence or presence of a mLight blocking LTBR-Ig fusion protein (50 ug/ml) and afterwards stained for intracellular Nr4a1 expression.

### Example 7: RNA and Protein Expression Analysis of Tregs Stimulated with AIRs

In this experiment it was tested if Tregs stimulated with AIRs according to the present disclosure show an expression patters, as measured by RNA sequence expression analysis, that is consistent with the postulated TCR signaling effect.

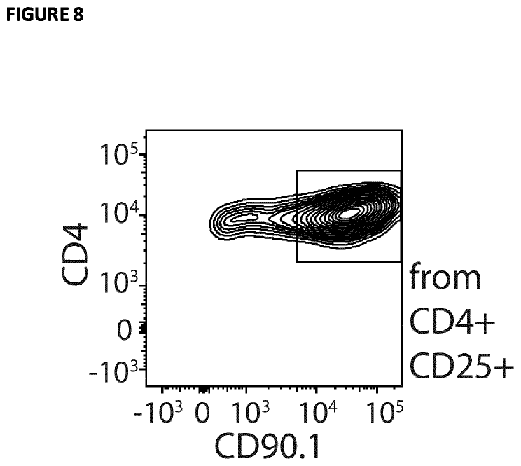

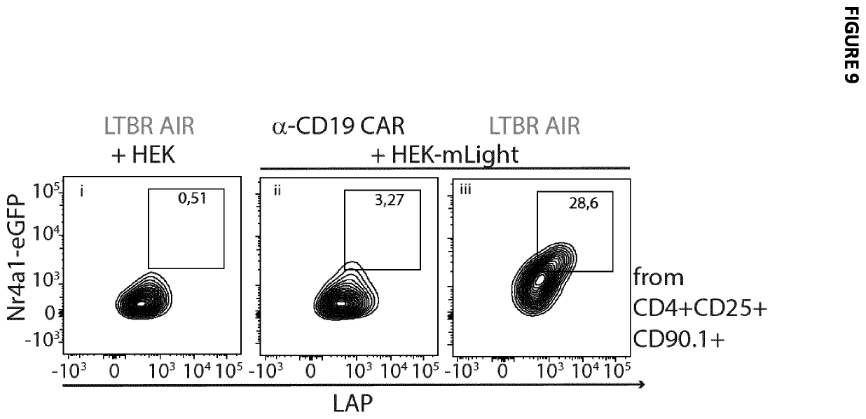

First cells were sorted accordingly. Tregs expressing an AIR or an irrelevant CAR carrying extracellularly an anti-CD19 single chain antibody (scFv) were rested for 24 h and then co-cultured for 18 h with HEK cells or HEK cells expressing mLight. FIG. 8 shows the pre-gating for CD90.1+ cells. Pre-gated cells were then sorted on CD4+CD25+CD90.1+ (left and middle) or CD4+CD25+CD90.1+Nr4a1+LAP+ (right). Results are shown in FIG. 9.

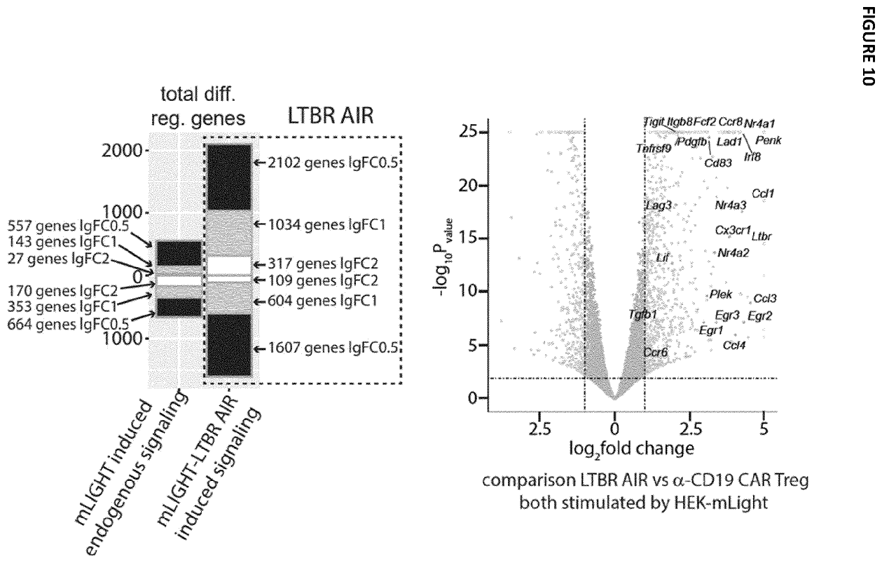

After cell sorting, RNA was isolated and RNA sequencing was performed. Results are shown in FIGS. 10 (LTBR AIR), 11(DR3 AIR) and 12 (α-CD19 control CAR).

Shown on the left in FIG. 10 is a summary of up and down regulated genes comparing α-CD19 CAR Treg stimulated with HEK-mLight vs. LTBR AIR Treg stimulated with HEK cells as control (endogenous mLight signaling) and LTBR AIR Treg stimulated with HEK-mLight vs. α-CD19CAR Treg stimulated with HEK-mLight (specific LTBR AIR signaling). Shown on the right is a Volcano plot showing differentially expressed transcripts.

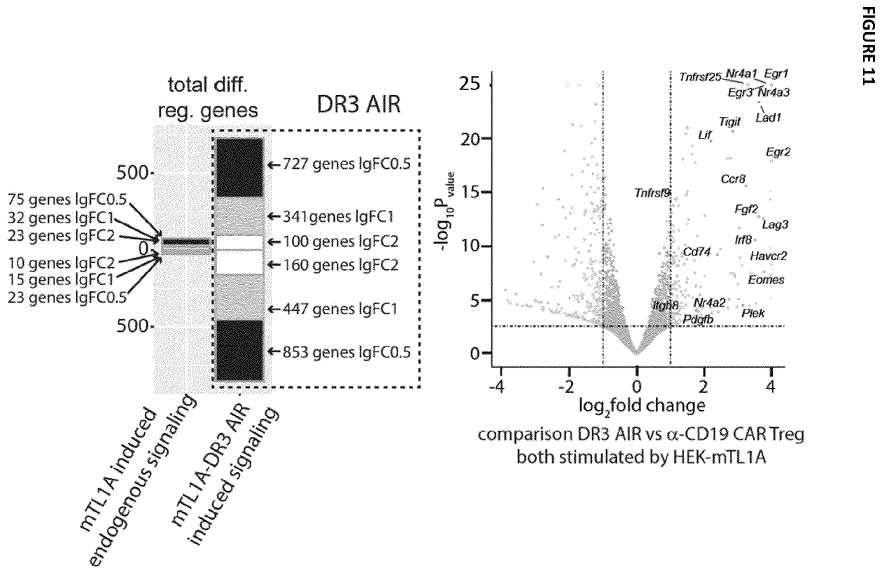

Similarly, in FIG. 11 DR3 AIR Treg's were stimulated with HEK cells (endogenous mTL1A signaling). DR3 AIR Treg were stimulated with HEK-mTL1A vs. α-CD19 CAR Treg stimulated with HEK-mTL1A (specific DR3 AIR signaling). Differentially expressed gene are shown on the left, a Volcano plot is shown on the right.

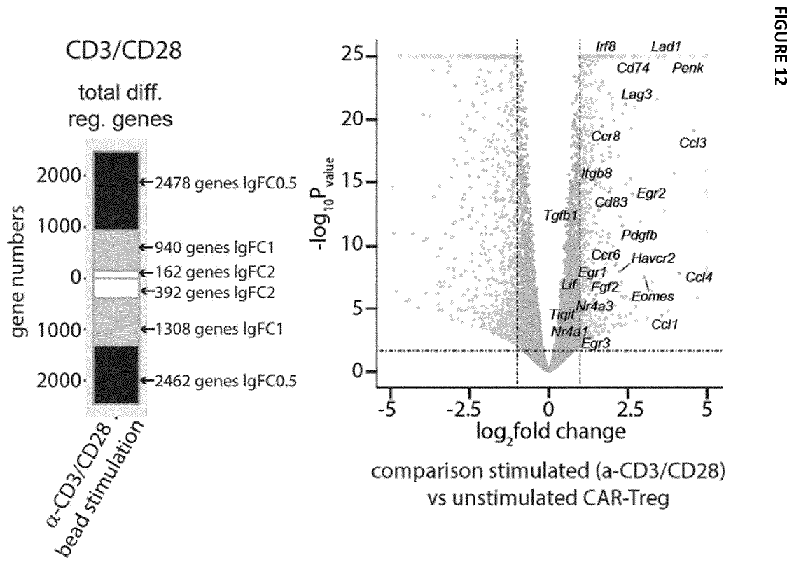

Also similarly, FIG. 12 compares an α-CD19 CAR Treg stimulated with anti-CD3/CD28 beads vs. unstimulated Treg. Differentially expressed gene are shown on the left, a Volcano plot is shown on the right.

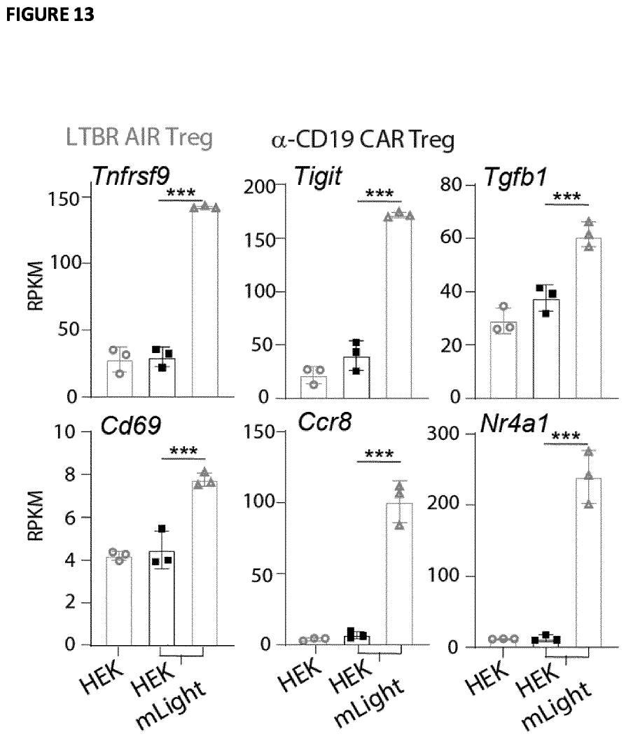

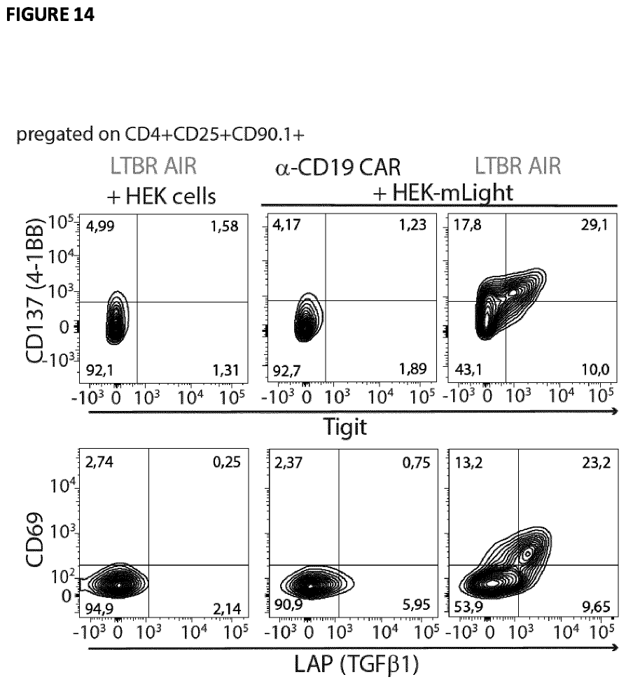

The RNA and or protein expression levels of various specific marker genes were analyzed in more detail. FIG. 13 shows the RNA expression data from LTBR AIR or control CAR expressing Tregs for Tnfrsf9, Tigit, Tgfb1, Cd69, Ccr8 and Nr4a1 after 18 h co-culture with HEK+/−mLight (Deseq2, n=3). LTBR AIRs (in each of the graphs shown on the very right) lead to a significant upregulation of all six marker genes tested. FIG. 14 shows a representative flow cytometry analysis from LTBR AIR or control CAR expressing Tregs after 18 h co-culture with HEK+/−mLight. Protein expression of CD137 and Tigit is shown in the upper, CD69 and LAP (membrane-bound Tgfβ1) in the lower panel. Data is representative for three independent experiments. Again the LTBR AIR leads to a significant upregulation of the marker genes investigated.

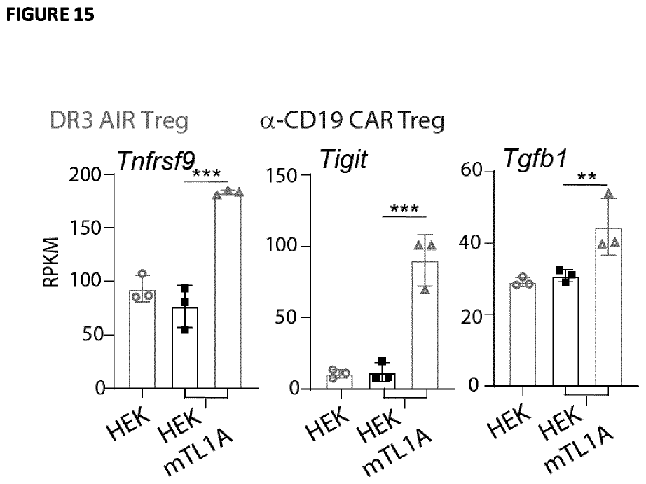

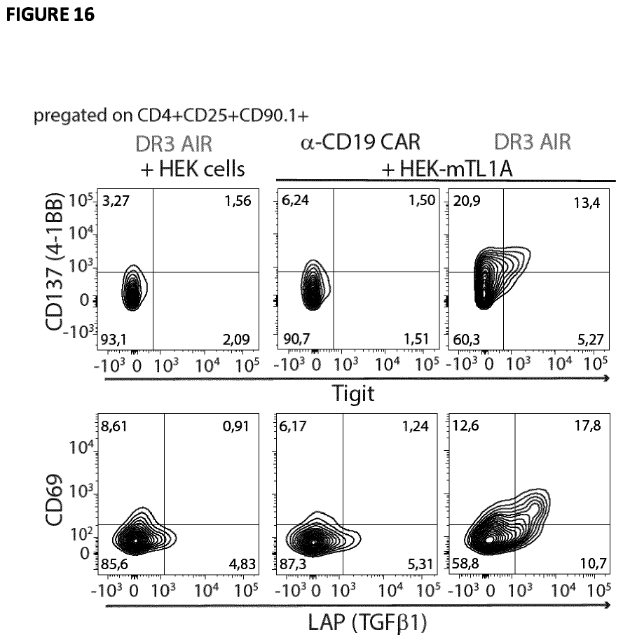

FIG. 15 shows RNA expression data from DR3 AIR or control CAR expressing Treg for Tnfrsf9, Tigit, Tgfb1 after 18 h co-culture with HEK+/−mLight (Deseq2, n=3). DR3 AIRs (in each of the graphs shown on the very right) lead to a significant upregulation of all three marker genes tested. FIG. 16 shows a representative flow cytometry analysis from DR3 AIR or control CAR expressing Treg after 18 h co-culture with HEK+/−mTL1A. Protein expression of CD137 and Tigit is shown in the upper, CD69 and LAP (Tgfβ1) in the lower panel. Data is representative for three independent experiments. Again the DR3 AIR leads to a significant upregulation of the marker genes investigated.

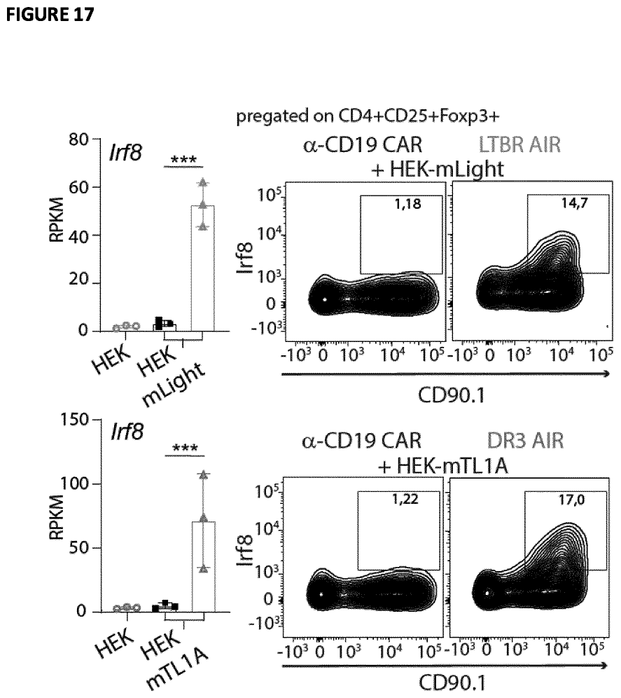

FIG. 17 shows RNA expression data (upper left, Deseq2, n=3) and protein expression (right) from LTBR AIR or control CAR expressing Treg for Irf8 after 18 h co-culture with HEK+/−mLight. RNA expression data (lower left, Deseq2, n=3) and protein expression (right) from DR3 AIR or control CAR expressing Treg for Irf8 after 18 h co-culture with HEK+/−mTL1A. Again, all marker genes tested are significantly upregulated.

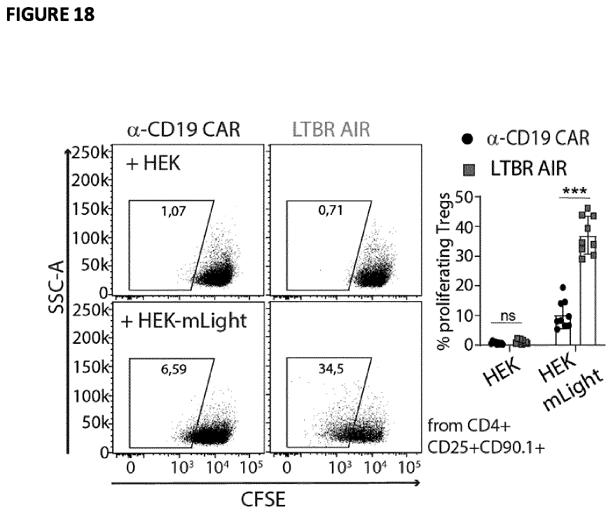

FIG. 18 demonstrates that the AIRs of the present disclosure mediate Treg activation and proliferation. LTBR AIR or control CAR expressing Treg were rested for 24 h and labeled with CFDA-SE proliferation dye. Engineered and labeled Treg were co-cultured with HEK+/−mLight for 72 h in presence of IL2 and afterwards analyzed with flow cytometry for proliferation. Representative dot plots are shown left. Summarized data from three experiments performed in three technical replicates are shown right (n=3). Yet again, all marker genes tested are significantly upregulated.

In summary, the RNA and protein expression analyses clearly indicate that the AIRs of the present disclosure elicit a cellular response in line with the underlying hypothesis.

### Example 8: LTBR AIR Ameliorates GvHD Pathology in Mice

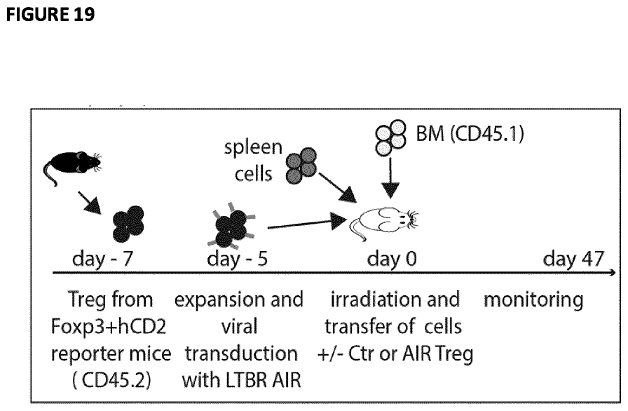

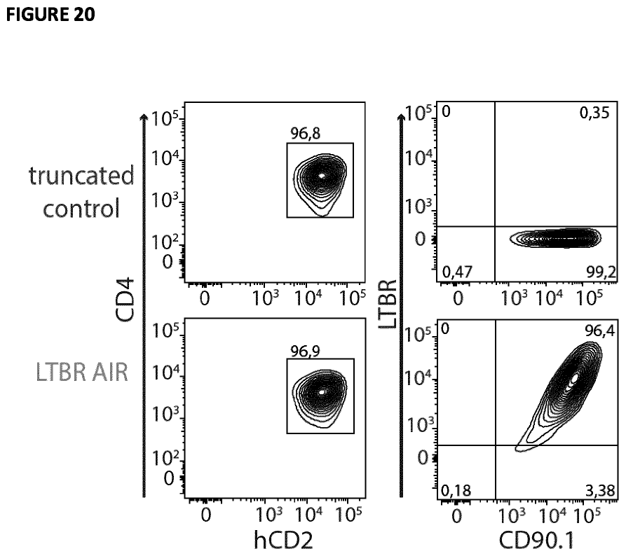

Usefulness of the AIRs of the present disclosure was tested in a Graft versus Host Diseases (GvHD) mouse model. FACS sorted Treg from Foxp3-hCD2 reporter mice were expanded and transduced with LTBR AIR or a truncated construct (lacking an extracellular binding domain, shown in FIG. 1 a) as control. 2.5×10⁵ engineered Treg were transplanted together with 2.5×10⁶ bone marrow cells and 2.5×10⁵ spleen cells from C57/BL6 mice into lethally irradiated (8 Gy) Balb/c mice. As transplant control, one group received only bone marrow cells (BM) and as disease developing control, on group received bone marrow cell plus spleen cells. After transplantation, animals were monitored for 47 days. An overview of the experiment is depicted in FIG. 19. The quality of the engineered Tregs were checked prior to transfer into mice by staining for CD4, hCD2 (reporter for Foxp3) as well as CD90.1 and LTBR before transfer into mice. Results are shown in FIG. 20. On day 20 a blood sample from each animal was taken to verify for donor derived (H2 kB+) CD19+ B-cell recovery.

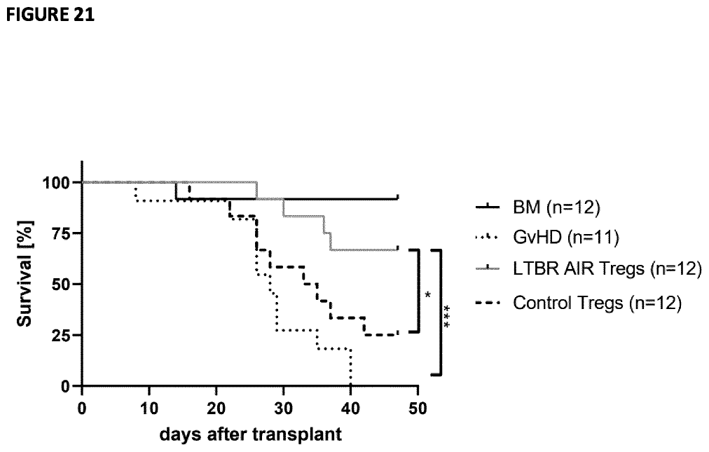

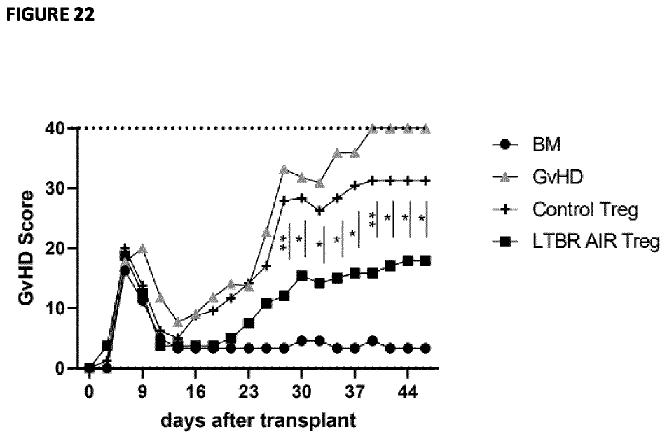

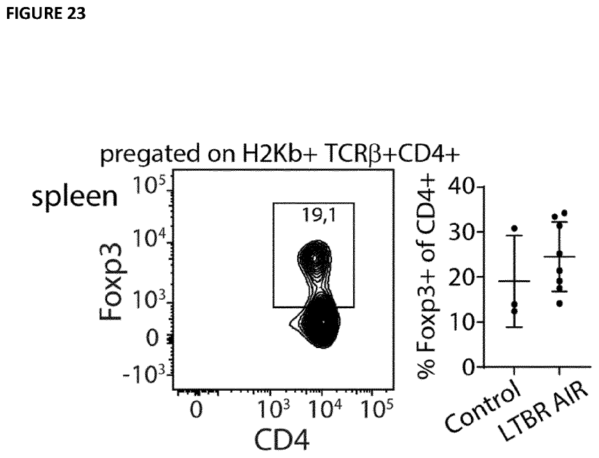

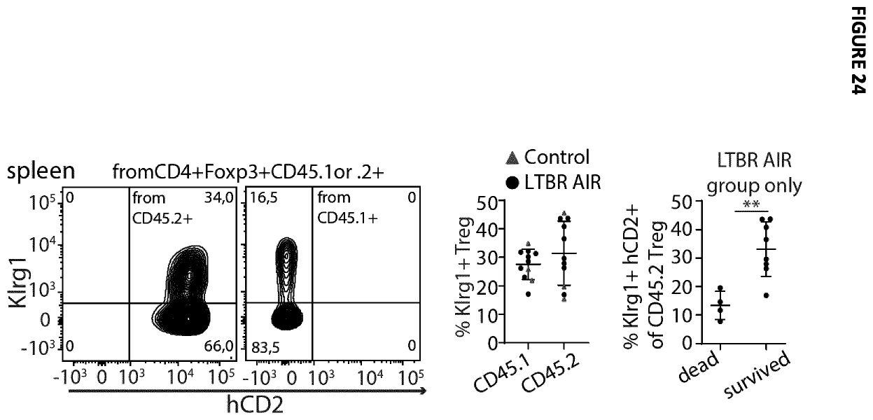

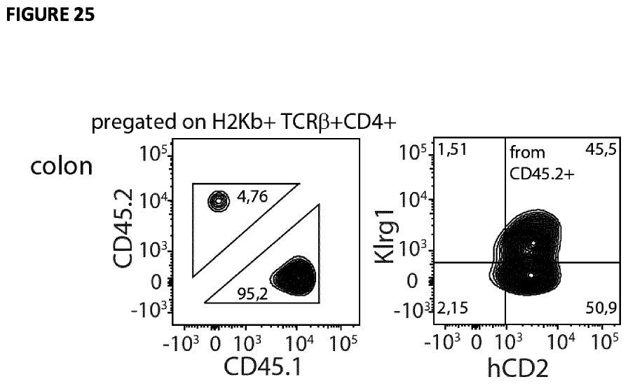

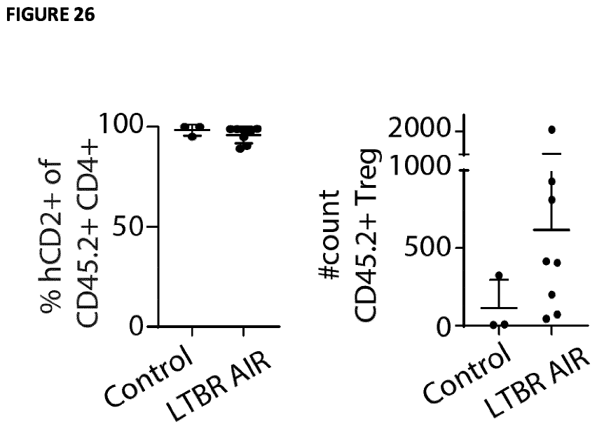

FIG. 21 shows a Kaplan-Meier plot containing data set from two independent experiments (log rank test, n=11-12). As can be seen the LTBR AIR was highly efficient in the GvHD model, leading to a significantly increased survival of transplanted Balb/c mice. This can also be seen in FIG. 22, which shows the mean GvHD score per treatment group (animals reaching a score of 40 had to be euthanized; dead animals kept the highest score over following time points for statistical analysis, two-way ANOVA). FIG. 23 shows a representative FACS plot (left) of a spleen of a surviving LTBR AIR Treg receiving animal. Shown on the right is the percentage of Foxp3+ Tregs in spleen of surviving mice at day 47. FIG. 24 shows the analysis of KIrg1+ Tregs in spleens. Shown on the left is a representative FACS plot of discriminated CD45.1+ (BM derived) and CD45.2+ (transferred AIR Treg) Treg cells. KIrg1 is a marker for the tissue specific phenotype; hCD2 is a Foxp3 reporter. Shown in the middle is the KIrg1+ expression of engineered CD45.2+ and BM derived CD45.1+ Treg in surviving mice. Shown on the right frequency of KIrg1+hCD2+ Tregs in mice receiving LTBR AIR Treg (Mann-Whitney-U, survivors n=8 vs. dead n=4). FIG. 25 shows a representative FACS analysis of a digested colon of a surviving LTBR AIR Treg receiving animal. Shown is discrimination between CD45.1 and CD45.2 cells within the CD4+ population on the left. CD45.2 Treg are further analyzed for KIrg1 and hCD2 expression on the right. FIG. 26 shows the percentage of hCD2+ (indicating Foxp3+) cells among transferred engineered CD45.2+ cells (left), demonstrating the phenotypic stability over 47 day in vivo. Shown on the right is the cell count of transferred CD45.2 Treg in colon at day 47.

Taken together, this example demonstrates the clinical usefulness of the AIRs of the present disclosure in an established clinical model for Graft versus Host Diseases.

### Example 9: Human LTBR AIR

A human LTBR AIR construct was generated as similar to the murine LTBR construct. The human construct contains the same elements as the murine construct, except that all domains, i.e. the LTBR extracellular domain, the LTBR transmembrane domain, the cytoplasmic CD28 domain and the cytoplasmic CD3zeta domain are of human origin (SEQ ID No. 30 (nucleic acid); SEQ ID No. 31 (polypeptide)). Expression and the signaling capacity of the human LTBR AIR construct was tested.

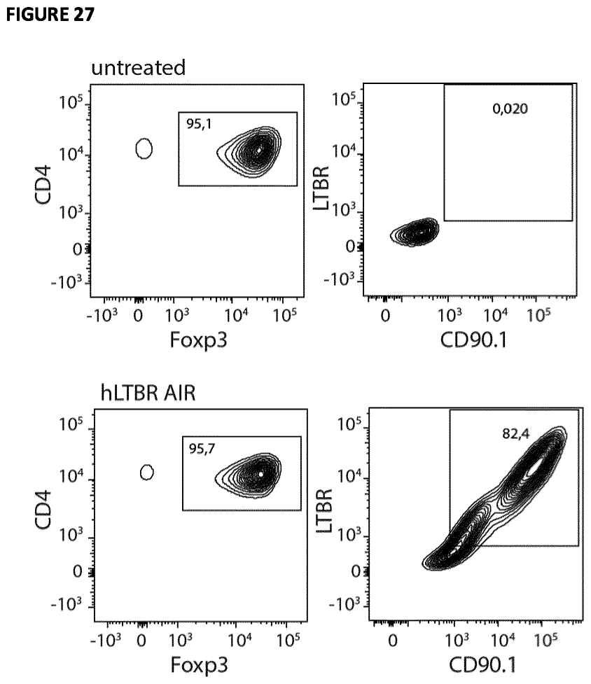

Human T cells from healthy blood donors were sorted on Treg protein markers (TCRb+CD4+CD25+CD127-CD45RA+) and expanded with anti-CD3/CD28 stimulation (TransACT, Miltenyi Biotec, Bergisch Gladbach, Germany) and IL-2 (500 U/ml) for 7 days. On day 2 after isolation Treg were transduced with the human LTBR AIR construct (using an amphotropic version of the retroviral expression system MSCV). 5 days later cultured Treg were stained for intracellular Foxp3+ to check for Treg purity and for CD90.1 as well as hLTBR to analyze transduction efficacy and surface expression of the AIR protein. Results are shown in FIG. 27. LTBR AIR expression on human Treg cells could be verified.

In another experiment, Tregs expressing the human LTBR AIR or an irrelevant CAR (an anti-CEA, Carcinoembrionic Antigen) were co-cultured for 18 h with parental HEK cells or HEK cells expressing human Light protein on the surface. Expression of Ccr8, Glycoprotein A repetitions predominant (GARP), CD137 (4-1BB) and Tigit is shown in CD90.1+Foxp3+ Tregs. Again, the expression levels of all marker genes confirm the postulated activity of the human LTBR AIR.

### Example 10: TCR Signaling is Important for the Activity of the AIRs

Control constructs were generated that are functional comparable to the AIRs of the present disclosure, with the important difference that they are devoid of the CD3zeta TCR signaling domain. Such constructs without a CD3zeta domain are known in the prior art (see WO2021/051195). Unlike the constructs of WO2021/051195, the constructs provided by the present invention exert their function directly at the main signal of the signaling cascade and in a CD3 (TCR)-dependent manner.

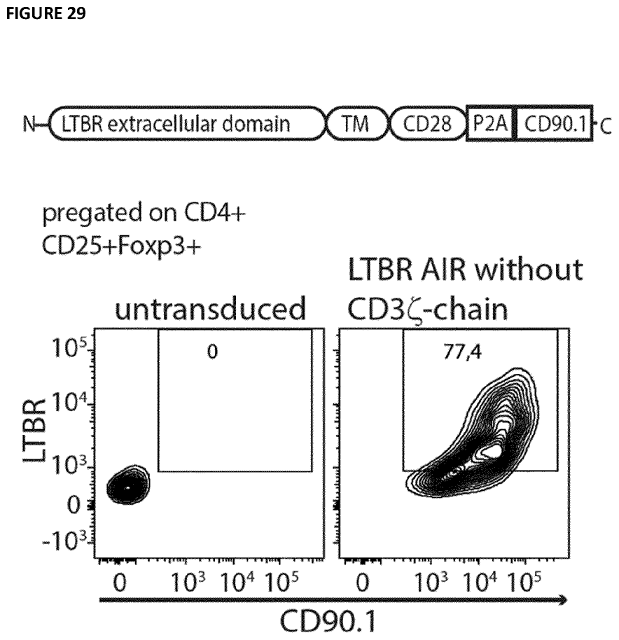

The architecture of the construct is shown on the top of FIG. 29. The amino acid sequence of this construct is depicted below:

(SEQ ID NO.: 48)
MRLPRASSPCGLAWGPLLLGLSGLLVASQPQLVPPYRIENQTCWDQDKEYYEPMHDVCCSRCPPGEFV
FAVCSRSQDTVCKTCPHNSYNEHWNHLSTCQLCRPCDIVLGFEEVAPCTSDRKAECRCQPGMSCVYLD
NECVHCEEERLVLCQPGTEAEVTDEIMDTDVNCVPCKPGHFQNTSSPRARCQPHTRCEIQGLVEAAPG
TSYSDTICKNPPEPGAMLLLAILLSLVLFLLFTTVLACAWAAANSRRNRGGQSDYMNMTPRRPGLTRK
PYQPYAPARDFAAYRPID

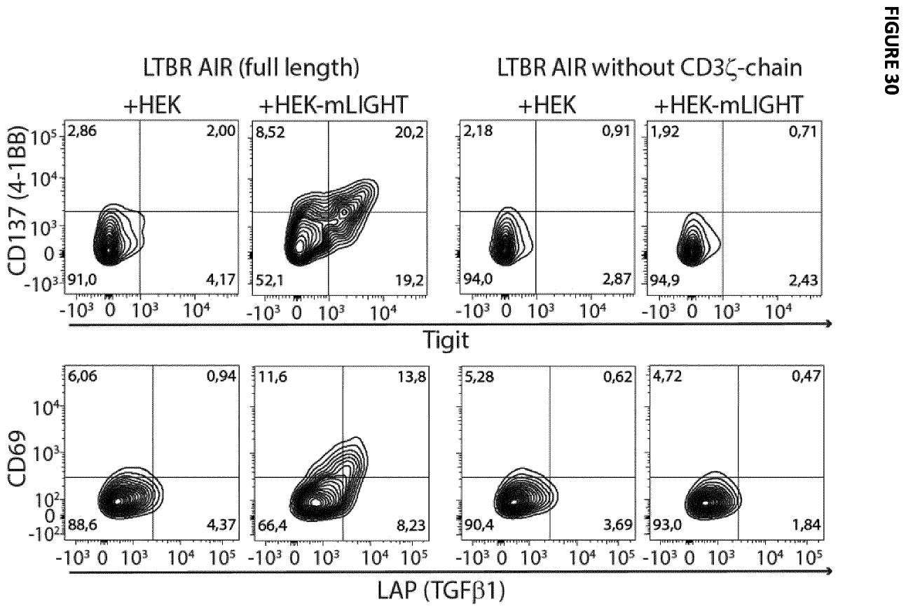

Surface expression of the construct could be demonstrated by flow cytometry. See FIG. 29, bottom. However, unlike the AIRs of the present disclosure, the construct which is devoid of CD3zeta does not induce the marker genes. The AIRs of the present disclosure are hence functionally different from the molecules of the prior art. FIG. 30 shows a representative flow cytometry analysis of Tregs expressing a full length LTBR AIR with a LTBR “AIR” lacking the CD3zeta-chain after 18 h co-culture with HEK+/−mLight. Protein expression of CD137 and Tigit is shown in the upper, CD69 and LAP (membrane-bound Tgfβ1) in the lower panel.

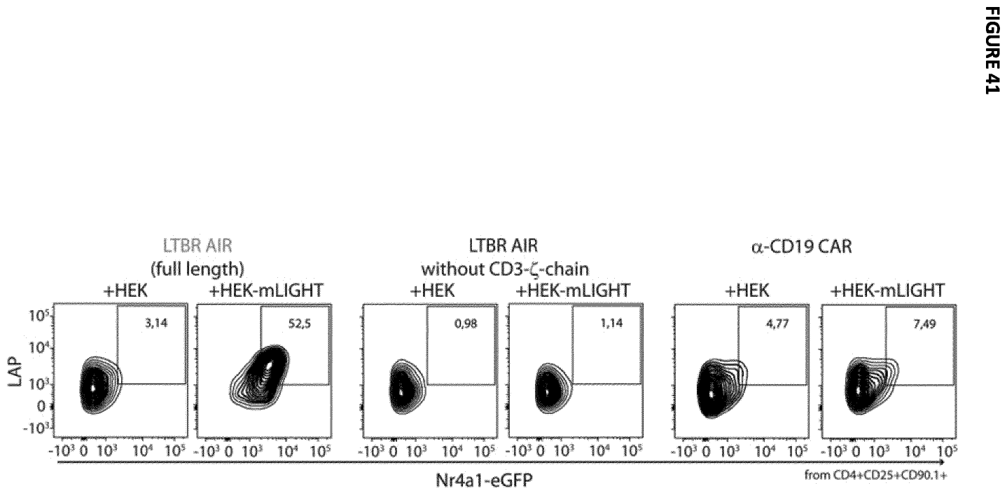

The importance of the CD3 zeta domain was further demonstrated with a LTBR-AIR, aLTBR-AIR without the CD3 zeta chain, and anti-CD19 CAR-expressing Treg cells from Nr4a1.eGFP reporter mice. Cells were rested for 24 h and then cocultured with HEK cells or HEK cells expressing mLIGHT for 18 h. Afterward cells were analyzed for eGFP and LAP expression via flow cytometry. Results of one of two independent experiments are shown in FIG. 41. Only the full length LTBR AIR containing the CD3zeta domain, but not the other two constructs tested, is capable to induce Nr4a1.eGFP upregulation in response to the corresponding ligand, mLIGHT. This finding demonstrates again the importance of the CD3 domain for the AIR mediated TCR-like signaling and Treg cell activation.

### Example 11: CD40 AIR Leads to an Expression Functionally Relevant Marker Genes

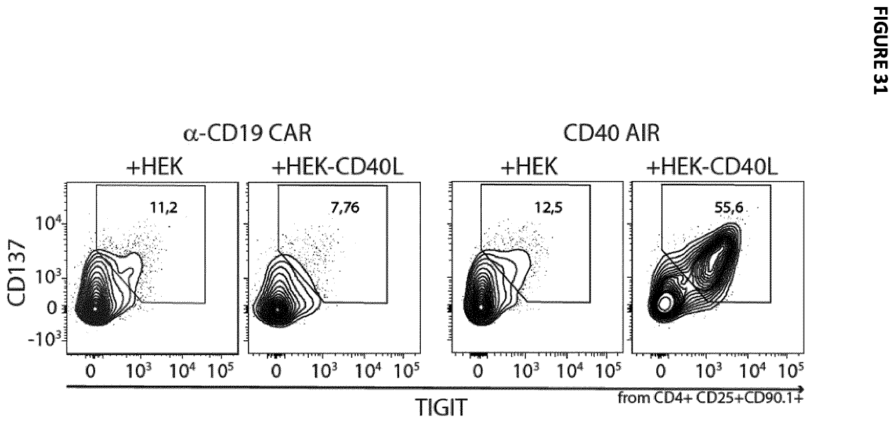

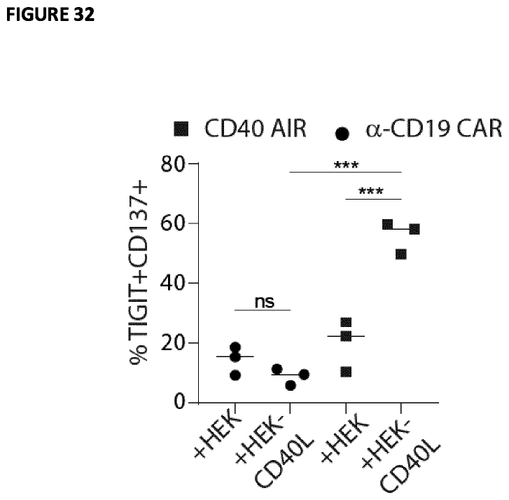

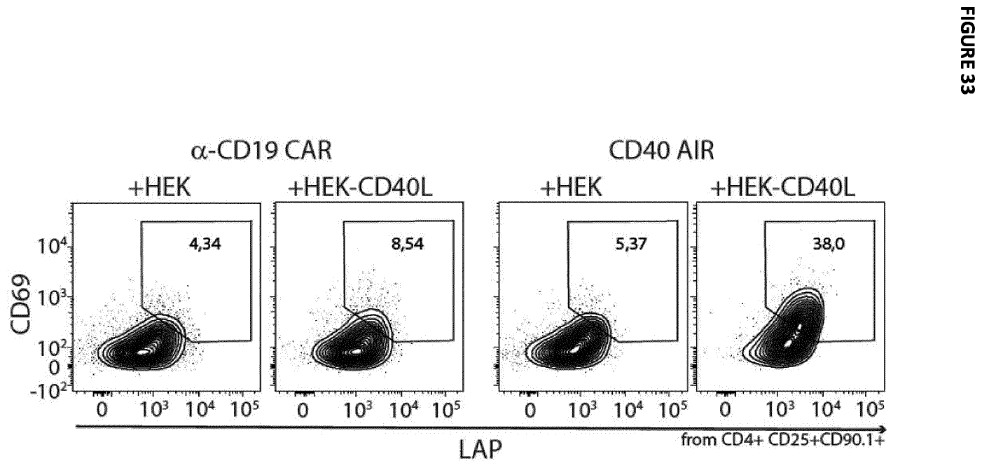

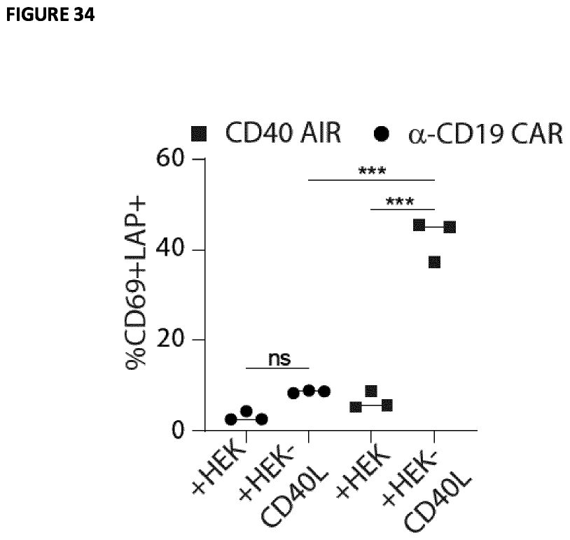

A similar experiment was also performed with a murine CD40 AIR of SEQ ID No. 43. This AIR was tested in comparison to a construct containing an extracellular anti-CD19 single chain antibody (scFv) instead of a binding domain of a Tumor-Necrosis-Factor (TNF) superfamily member (construct F of FIG. 1). The CD40 AIR or the anti-CD19 construct expressing Treg cells were co-cultured for 18 h with HEK cells or HEK cells expressing CD40L (CD154). Afterwards cells were analyzed via flow cytometry for expression of TIGIT and CD137 (FIGS. 31 and 32), as well as LAP (TGFb1) and CD69 (FIGS. 33 and 34).

Again, it could be shown that the AIRs of the present disclosure are functionally active and induce the expected marker genes (FIGS. 31 and 33). Results are summarized in FIGS. 32 and 34. The induction of the marker genes by the CD40 AIR is statistically significant as determined by a one-way Anova analysis.

### Example 12: CD40 AIR Triggers the Expression of Nr4a1, an Early Response Gene of TCR Signaling

Treg cells from Nr4a1-eGFP reporter mice expressing the constructs of Example 11 were cocultured for 18 h with HEK cells or HEK cells expressing CD40L (CD154). Afterwards expression of Nr4a1-eGFP was measured by flow cytometry.

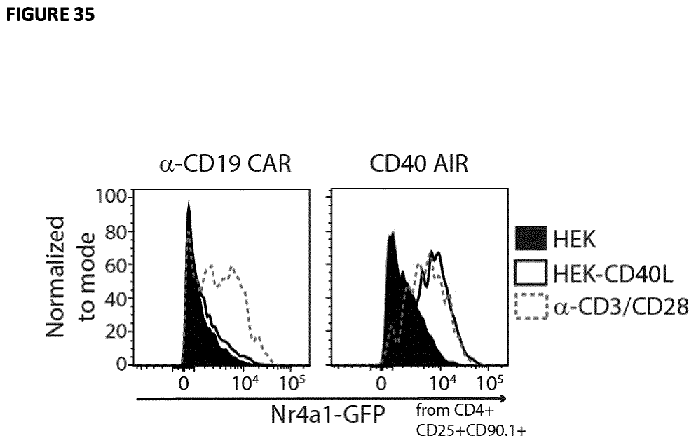

The expression of Nr4a1, an early response gene of TCR signaling, was induced by Treg cells expressing the CD40 AIR, but not by the construct expressing the anti-CD19 CAR (FIG. 35), again confirming the TCR-like activation mediated by the AIRs of the present disclosure.

### Example 13: Design of AIRs with Alternative Costimulatory Domains

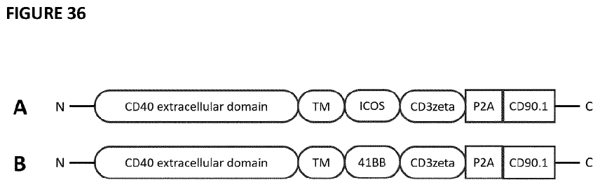

In order to test if alternative costimulatory domains are also functional, additional constructs were generated. To do so, the CD28 costimulatory domain of the construct shown in FIG. 1G was replaced by the ICOS costimulatory domain and the 4-1BB costimulatory domain. These additional constructs are shown in FIG. 36A (ICOS) and 36B (4-1BB). Sequences of the constructs and their parts are shown in Table 6 (N=nucleic acid, P=polypeptide). The CD90.1 marker gene is cut off via the P2A cleavage site and is not part of the AIR. Constructs were generated as outlined in Example 4.

TABLE 6

| SEQ ID | | | |
|---|---|---|---|
| No. | Description | N/P | Sequence |
| 49 | CD40, complete | N | ATGGTGTCTTTGCCTCGGCTGTGCGCGCTATGGGGCTGCTT |
| | AIR with ICOS | | GTTGACAGCGGTCCATCTAGGGCAGTGTGTTACGTGCAGTG |
| | costim, mouse | | ACAAACAGTACCTCCACGATGGCCAGTGCTGTGATTTGTGC |
| | | | CAGCCAGGAAGCCGACTGACAAGCCACTGCACAGCTCTTGA |
| | | | GAAGACCCAATGCCACCCATGTGACTCAGGCGAATTCTCAG |
| | | | CCCAGTGGAACAGGGAGATTCGCTGTCACCAGCACAGACAC |
| | | | TGTGAACCCAATCAAGGGCTTCGGGTTAAGAAGGAGGGCAC |
| | | | CGCAGAATCAGACACTGTCTGTACCTGTAAGGAAGGACAAC |
| | | | ACTGCACCAGCAAGGATTGCGAGGCATGTGCTCAGCACACG |
| | | | CCCTGTATCCCTGGCTTTGGAGTTATGGAGATGGCCACTGA |
| | | | GACCACTGATACCGTCTGTCATCCCTGCCCAGTCGGCTTCT |
| | | | TCTCCAATCAGTCATCACTTTTCGAAAAGTGTTATCCCTGG |
| | | | ACAAGCTGTGAGGATAAGAACTTGGAGGTCCTACAGAAAGG |
| | | | AACGAGTCAGACTAATGTCATCTGTGGTTTAAAGTCCCGGA |
| | | | TGCGAGCCCTGCTGGTCATTCCTGTCGTGATGGGCATCCTC |
| | | | ATCACCATTTTCGGGGTGTTTCTCTATATCtCAAAAAAGAA |
| | | | ATACGGATCTAGTGTGCATGACCCTAATAGTGAATACATGT |
| | | | TCATGGCGGCAGTCAACACAAACAAAAAGTCTAGACTTGCA |
| | | | GGTGTGACCTCAAGAGCCAAGTTCAGCAGATCCGCCGAGAC |
| | | | AGCCGCCAACCTGCAGGATCCCAACCAGCTGTACAACGAGC |
| | | | TGAACCTGGGCAGACGGGAGGAATACGACGTGCTGGAAAAG |
| | | | AAGAGAGCCAGGGACCCCGAGATGGGCGGCAAGCAGCAGAG |
| | | | AAGAAGAAACCCTCAGGAAGGCGTCTACAACGCCCTGCAGA |
| | | | AAGACAAGATGGCCGAGGCCTACAGCGAGATCGGCACCAAG |
| | | | GGCGAGAGAAGAAGGGGCAAGGGCCACGATGGCCTGTACCA |
| | | | GGGCCTGTCCACCGCCACCAAGGACACCTACGACGCCCTGC |
| | | | ACATGCAGACCCTGGCCCCCAGA |
| 50 | CD40, complete | P | MVSLPRLCALWGCLLTAVHLGQCVTCSDKQYLHDGQCCDLC |
| | AIR with ICOS | | QPGSRLTSHCTALEKTQCHPCDSGEFSAQWNREIRCHQHRH |
| | costim, mouse | | CEPNQGLRVKKEGTAESDTVCTCKEGQHCTSKDCEACAQHT |
| | | | PCIPGFGVMEMATETTDTVCHPCPVGFFSNQSSLFEKCYPW |
| | | | TSCEDKNLEVLQKGTSQTNVICGLKSRMRALLVIPVVMGIL |
| | | | ITIFGVFLYISKKKYGSSVHDPNSEYMEMAAVNTNKKSRLA |
| | | | GVTSRAKFSRSAETAANLQDPNQLYNELNLGRREEYDVLEK |
| | | | KRARDPEMGGKQQRRRNPQEGVYNALQKDKMAEAYSEIGTK |
| | | | GERRRGKGHDGLYQGLSTATKDTYDALHMQTLAPR |
| 51 | CD40, complete | N | ATGGTGTCTTTGCCTCGGCTGTGCGCGCTATGGGGCTGCTT |
| | AIR with 4-1BB | | GTTGACAGCGGTCCATCTAGGGCAGTGTGTTACGTGCAGTG |
| | costim, mouse | | ACAAACAGTACCTCCACGATGGCCAGTGCTGTGATTTGTGC |
| | | | CAGCCAGGAAGCCGACTGACAAGCCACTGCACAGCTCTTGA |
| | | | GAAGACCCAATGCCACCCATGTGACTCAGGCGAATTCTCAG |
| | | | CCCAGTGGAACAGGGAGATTCGCTGTCACCAGCACAGACAC |
| | | | TGTGAACCCAATCAAGGGCTTCGGGTTAAGAAGGAGGGCAC |
| | | | CGCAGAATCAGACACTGTCTGTACCTGTAAGGAAGGACAAC |
| | | | ACTGCACCAGCAAGGATTGCGAGGCATGTGCTCAGCACACG |
| | | | CCCTGTATCCCTGGCTTTGGAGTTATGGAGATGGCCACTGA |
| | | | GACCACTGATACCGTCTGTCATCCCTGCCCAGTCGGCTTCT |
| | | | TCTCCAATCAGTCATCACTTTTCGAAAAGTGTTATCCCTGG |
| | | | ACAAGCTGTGAGGATAAGAACTTGGAGGTCCTACAGAAAGG |
| | | | AACGAGTCAGACTAATGTCATCTGTGGTTTAAAGTCCCGGA |
| | | | TGCGAGCCCTGCTGGTCATTCCTGTCGTGATGGGCATCCTC |
| | | | ATCACCATTTTCGGGGTGTTTCTCTATATCAAATGGATCAG |
| | | | GAAAAAATTCCCCCACATATTCAAGCAACCATTTAAGAAGA |
| | | | CCACTGGAGCAGCTCAAGAGGAAGATGCTTGTAGCTGCCGA |
| | | | TGTCCACAGGAAGAAGAAGGAGGAGGAGGAGGCTATGAGCT |
| | | | GAGAGCCAAGTTCAGCAGATCCGCCGAGACAGCCGCCAACC |
| | | | TGCAGGATCCCAACCAGCTGTACAACGAGCTGAACCTGGGC |
| | | | AGACGGGAGGAATACGACGTGCTGGAAAAGAAGAGAGCCAG |
| | | | GGACCCCGAGATGGGCGGCAAGCAGCAGAGAAGAAGAAACC |
| | | | CTCAGGAAGGCGTCTACAACGCCCTGCAGAAAGACAAGATG |
| | | | GCCGAGGCCTACAGCGAGATCGGCACCAAGGGCGAGAGAAG |
| | | | AAGGGGCAAGGGCCACGATGGCCTGTACCAGGGCCTGTCCA |
| | | | CCGCCACCAAGGACACCTACGACGCCCTGCACATGCAGACC |
| | | | CTGGCCCCCAGA |
| 52 | CD40, complete | P | MVSLPRLCALWGCLLTAVHLGQCVTCSDKQYLHDGQCCDLC |
| | AIR with 4-1BB | | QPGSRLTSHCTALEKTQCHPCDSGEFSAQWNREIRCHQHRH |
| | costim, mouse | | CEPNQGLRVKKEGTAESDTVCTCKEGQHCTSKDCEACAQHT |
| | | | PCIPGFGVMEMATETTDTVCHPCPVGFFSNQSSLFEKCYPW |
| | | | TSCEDKNLEVLQKGTSQTNVICGLKSRMRALLVIPVVMGIL |
| | | | ITIFGVFLYIKWIRKKFPHIFKQPFKKTTGAAQEEDACSCR |
| | | | CPQEEEGGGGGYELRAKFSRSAETAANLQDPNQLYNELNLG |
| | | | RREEYDVLEKKRARDPEMGGKQQRRRNPQEGVYNALQKDKM |
| | | | AEAYSEIGTKGERRRGKGHDGLYQGLSTATKDTYDALHMQT |
| | | | LAPR |
| 53 | CD40, complete | P | MVRLPLQCVLWGCLLTAVHPEPPTACREKQYLINSQCCSLC |
| | AIR with ICOS | | QPGQKLVSDCTEFTETECLPCGESEFLDTWNRETHCHQHKY |
| | costim, human | | CDPNLGLRVQQKGTSETDTICTCEEGWHCTSEACESCVLHR |
| | | | SCSPGFGVKQIATGVSDTICEPCPVGFFSNVSSAFEKCHPW |
| | | | TSCETKDLVVQQAGTNKTDVVCGPQDRLRALVVIPIIFGIL |
| | | | FAILLVLVFICWLTKKKYSSSVHDPNGEYMFMRAVNTAKKS |
| | | | RLTDVTLLRVKFSRSADAPAYQQGQNQLYNELNLGRREEYD |
| | | | VLDKRRGRDPEMGGKPRRKNPQEGLYNELQKDKMAEAYSEI |
| | | | GMKGERRRGKGHDGLYQGLSTATKDTYDALHMQALPPR |
| 54 | CD40, complete | P | MVRLPLQCVLWGCLLTAVHPEPPTACREKQYLINSQCCSLC |
| | AIR with 4-1BB | | QPGQKLVSDCTEFTETECLPCGESEFLDTWNRETHCHQHKY |
| | costim, human | | CDPNLGLRVQQKGTSETDTICTCEEGWHCTSEACESCVLHR |
| | | | SCSPGFGVKQIATGVSDTICEPCPVGFFSNVSSAFEKCHPW |
| | | | TSCETKDLVVQQAGTNKTDVVCGPQDRLRALVVIPIIFGIL |
| | | | FAILLVLVFIKRGRKKLLYIFKQPFMRPVQTTQEEDGCSCR |
| | | | FPEEEEGGCELLRVKFSRSADAPAYQQGQNQLYNELNLGRR |
| | | | EEYDVLDKRRGRDPEMGGKPRRKNPQEGLYNELQKDKMAEA |
| | | | YSEIGMKGERRRGKGHDGLYQGLSTATKDTYDALHMQALPP |
| | | | R |
| 55 | ICOS | P | SKKKYGSSVHDPNSEYMEMAAVNTNKKSRLAGVTS |
| | costimulatory | | |
| | domain, mouse | | |
| 56 | 4-1BB | P | KWIRKKFPHIFKQPFKKTTGAAQEEDACSCRCPQEEEGGGG |
| | costimulatory | | GYEL |
| | domain, mouse | | |
| 57 | ICOS | P | CWLTKKKYSSSVHDPNGEYMFMRAVNTAKKSRLTDVTL |
| | costimulatory | | |
| | domain, human | | |
| 58 | 4-1BB | P | KRGRKKLLYIFKQPFMRPVQTTQEEDGCSCRFPEEEEGGCE |
| | costimulatory | | L |
| | domain, human | | |

### Example 14: Surface Expression of CD40 AIR with ICOS and 41BB Costimulatory Domains

Transduction and surface expression of the AIRs with the ICOS and the 41BB costimulatory domains was tested as described in Example 5.

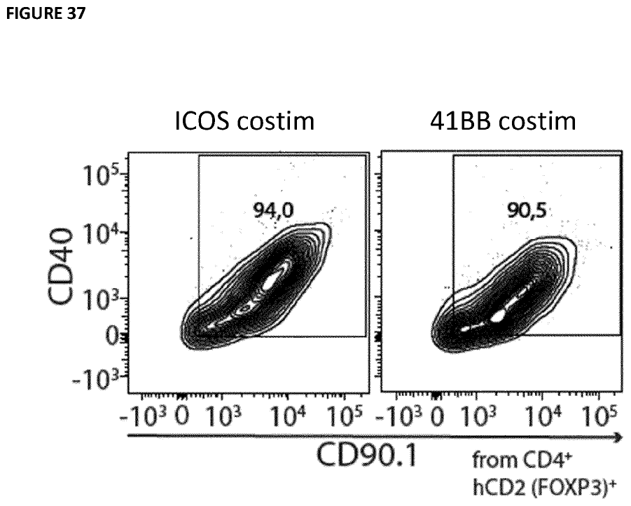

Transduction rates of the CD40 AIRs with different costimulatory domains (CD28, ICOS, 41BB) were comparable, as measured by flow cytometric analysis (data shown). Surface expression of the CD40 AIRs with the ICOS and the 41BB costimulatory domain are shown in FIG. 37. It could be demonstrated that, like the CD40 AIR with a CD28 costimulatory domain, also the CD40 AIR with an ICOS costimulatory domain or a 41BB costimulatory domain are efficiently expressed on the surface of regulatory T cells.

### Example 15: All Tested Costimulatory Domains Lead to Signaling of the CD40 AIR

In this experiment the signaling capability of the CD40 AIRs with the three different costimulatory domains was compared. Treg cells expressing CD40-AIR versions with a CD28, an ICOS or a 41BB costimulatory domain were rested for 24 h, and then cocultured with HEK cells or HEK cells expressing mCD40L for 18 h. Afterwards expression of CD69 and LAP was analyzed via flow cytometry (one way ANOVA).

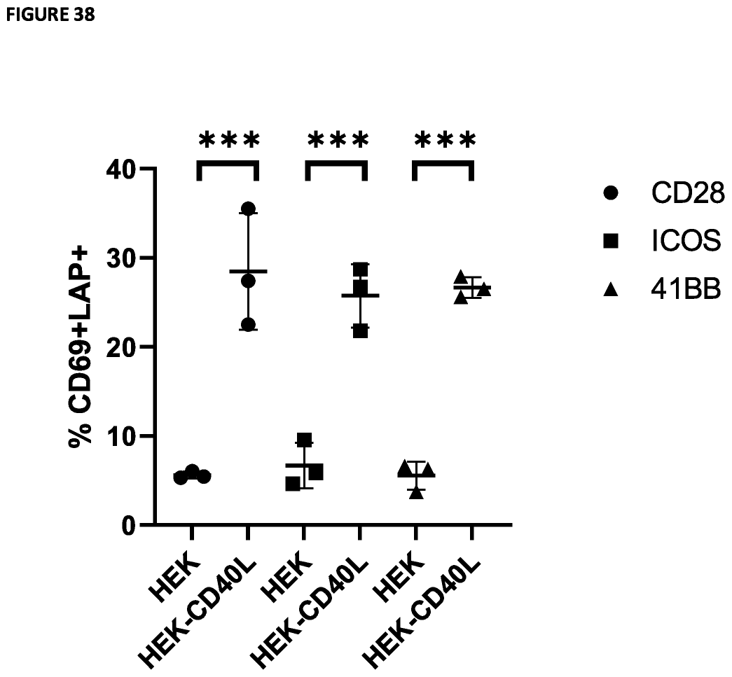

Results are shown in FIG. 38. All constructs tested show a similar capacity to induce Treg cell activation, confirming that any costimulatory domain can be used.

### Example 16: CD40 Ameliorates GvHD Pathology in Mice

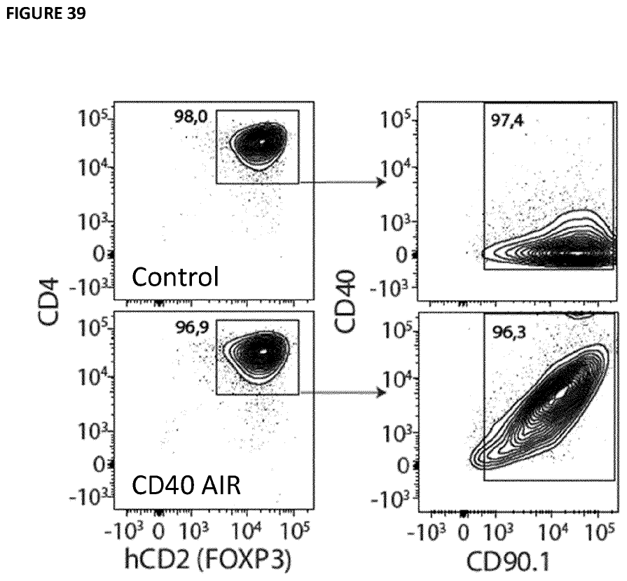

Like the LTBR AIR (see Example 8), also the CD40 AIR was tested for its therapeutic usefulness in a Graft versus Host Diseases (GvHD) mouse model. Prior to transplantation Treg cells were quality controlled by flow cytometric analysis for transduction efficiency (CD90.1 expression) and CD40 AIR surface expression on Treg cells three days after transduction. As a control, a construct lacking the extracellular CD40-binding domain was used). Results are shown in FIG. 39. Both construct, the CD40 AIR and the control construct, showed a high transduction efficiency, but only the cells transfected with the CD40AIR exhibited binding to CD40.

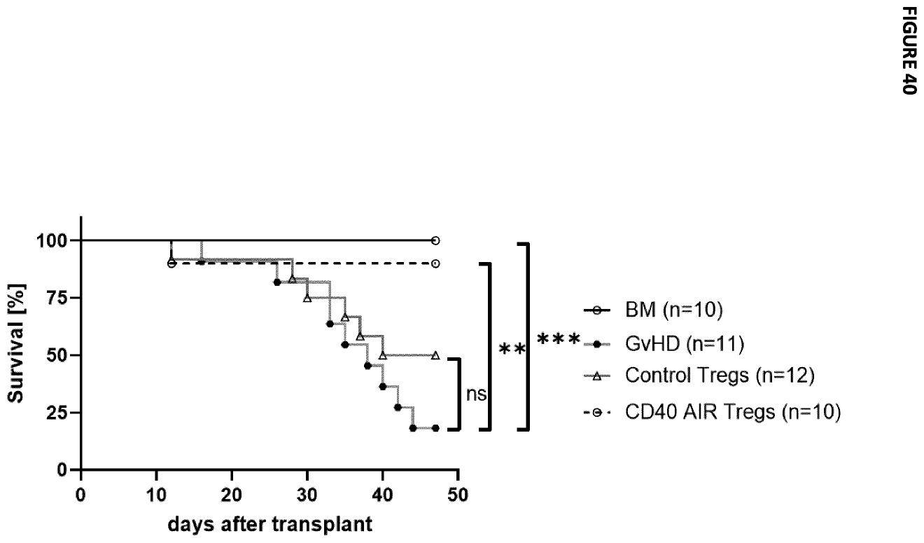

Results of the experiment are shown in FIG. 40. The Kaplan-Meier plot contains data set from two independent experiments (log rank test, n=10-12) and shows survival of transplanted BALB/c mice. As can be seen the CD40 AIR was highly efficient in the GvHD model, leading to a significantly increased survival of transplanted Balb/c mice. Taken together, this example validates the clinical usefulness of the AIRs of the present disclosure in an established clinical model for Graft versus Host Diseases.

1: An artificial immune receptor, comprising
a) an extracellular domain of a member of the tumor necrosis factor receptor superfamily,
b) a transmembrane domain,
c) a cytoplasmic costimulatory signaling domain, and
d) a cytoplasmic CD3 T cell receptor signaling domain.

2: An artificial immune receptor according to claim 1, wherein said member of the tumor necrosis factor receptor superfamily is selected from TNFR1, TNFR2, Fas, DR4, DR5, DR3, DR6, EDAR, XEDAR, TROY, LTBR, NGFR, CD18, CD134, CD40, CD27, CD30, CD137, TRAILR3, TRAILR4, CD265, osteoprotegerin, CD266, TACI, BAFF, BAFF receptor, APRIL, CD270, CD269 and CD357.

3: An artificial immune receptor according to claim 1, wherein said member of the tumor necrosis factor receptor superfamily is selected from TNFR2, DR3, LTBR and CD40.

4: An artificial immune receptor according to claim 1, wherein said transmembrane domain is from the same protein as the extracellular domain.

5: The artificial immune receptor according to claim 1, wherein said artificial immune receptor comprises the amino acid sequence of SEQ ID No.: 33, SEQ ID No.: 39, SEQ ID No.: 41 or SEQ ID No.: 46.

6: An artificial immune receptor according to claim 1, wherein said cytoplasmic costimulatory signaling domain is selected from 4-IBB (CD137), BAFFR, OX40, CD27, CD28, ICOS, CD40, 2B4, GITR, HVEM, OX40, RELT, TACI, TROY, TWEAK, KIR receptors, TLR1 to TLR9 receptors, IL-2, IL-7 and IL-15 receptors.

7: An artificial immune receptor according to claim 1, wherein said cytoplasmic costimulatory signaling domain is selected from 4-IBB (CD137), CD28 and ICOS.

8: An artificial immune receptor according to claim 1, wherein said cytoplasmic costimulatory signaling domain comprises the amino acid sequence of SEQ ID No.: 35.

9: An artificial immune receptor according to claim 1, wherein said cytoplasmic CD3 T cell receptor signaling domain is selected from CD3 zeta, CD3 gamma and CD3 epsilon, preferably CD3 zeta.

10: An artificial immune receptor according to claim 1, wherein said cytoplasmic T cell receptor signaling domain comprises the amino acid sequence of SEQ ID No.: 37.

11: An artificial immune receptor according to claim 1, wherein
a) said member of the tumor necrosis factor receptor superfamily and said transmembrane domain are from TNFR2, said cytoplasmic costimulatory signaling domain is CD28 and said cytoplasmic T cell receptor signaling domain is CD3 zeta,
b) said member of the tumor necrosis factor receptor superfamily and said transmembrane domain are from DR3, said cytoplasmic costimulatory signaling domain is CD28 and said cytoplasmic T cell receptor signaling domain is CD3 zeta,
c) said member of the tumor necrosis factor receptor superfamily and said transmembrane domain are from LTBR, said cytoplasmic costimulatory signaling domain is CD28 and said cytoplasmic T cell receptor signaling domain is CD3 zeta, or
d) said member of the tumor necrosis factor receptor superfamily and said transmembrane domain are from CD40, said cytoplasmic costimulatory signaling domain is CD28 and said cytoplasmic T cell receptor signaling domain is CD3 zeta.

12: A nucleic acid encoding an artificial immune receptor according to claim 1.

13: A vector comprising a nucleic acid according to claim 12.

14: A host cell comprising a nucleic acid according to claim 12.

15. (canceled)

16: A host cell comprising a vector according to claim 13.

17: A host cell expressing an artificial immune receptor according to claim 1.

18: A method for treating or preventing a disease or condition or ameliorating one or more symptoms thereof, said method comprising administering an artificial immune receptor according to claim 1, a nucleic acid encoding said artificial immune receptor, a vector comprising a nucleic acid encoding said artificial immune receptor, or a host cell expressing said artificial immune receptor or comprising a nucleic acid encoding said artificial immune receptor.

19: The method of claim 18 wherein the disease or condition is an inflammatory disorder or cancer.


SEQUENCE LISTING (58 sequences)

SEQ ID NO: 1 - DNA, 738 bp, Mus musculus
  source 1..738: mol_type = genomic DNA
atggggggctctctggtcagaagggatcagaagtctctccaagacagcagaaggatggcagaggagctggggttgggcttcggagaaggagtcccagtggaagtgctgccggaaggctgtagacacaggccagaggccagggccgggctagctgccaggagcaaagcctgcctggctctcacctgctgcctgttgtcatttcccatcctcgcaggacttagcaccctcctaatggctggccagctccgggtccccggaggcaagccgagagcacacctgacaattaagaaacaaaccccagcaccacatctgaaaaatcagctctctgctctacactgggaacatgacctagggatggccttcaccaagaacgggatgaagtacatcaacaaatccctggtgatcccagagtcaggagactatttcatctactcccagatcacattccgagggaccacatctgtgtgtggtgacatcagtcgggggagacgaccaaacaagccagactccatcaccatggttatcaccaaggtagcagacagctaccctgagcctgcccgcctactaacagggtccaagtctgtgtgtgaaataagcaacaactggttccagtccctctaccttggggccacgttctccttggaagaaggagacagactaatggtaaacgtcagtgacatctccttggtggattacacaaaagaagataaaactttctttggagctttcttgctataa

SEQ ID NO: 2 - DNA, 723 bp, Homo sapiens
  source 1..723: mol_type = genomic DNA
atggaggagagtgtcgtacggccctcagtgtttgtggtggatggacagaccgacatcccattcacgaggctgggacgaagccaccggagacagtcgtgcagtgtggcccgggtgggtctgggtctcttgctgttgctgatgggggccgggctggccgtccaaggctggttcctcctgcagctgcactggcgtctaggagagatggtcacccgcctgcctgacggacctgcaggctcctgggagcagctgatacaagagcgaaggtctcacgaggtcaacccagcagcgcatctcacaggggccaactccagcttgaccggcagcggggggccgctgttatgggagacccagctgggcctggccttcctgaggggcctcagctaccacgatggggcccttgtggtcaccaaagctggctactactacatctactccaaggtgcagctgggcggtgtgggctgcccgctgggcctggccagcaccatcacccacggcctctacaagcgcacaccccgctaccccgaggagctggagctgttggtcagccagcagtcaccctgcggacgggccaccagcagctcccgggtctggtgggacagcagcttcctgggtggtgtggtacacctggaggctggggaggaggtggtcgtccgtgtgctggatgaacgcctggttcgactgcgtgatggtacccggtcttacttcggggctttcatggtgtga

SEQ ID NO: 3 - DNA, 654 bp, Mus musculus
  source 1..654: mol_type = genomic DNA
atggagagtgtggtacagccttcagtgtttgtggtggatggacagacggacatcccattcaggcggctggaacagaaccaccggagacggcgctgtggcactgtccaggtcagcctggccctggtgctgctgctaggtgctgggctggccactcagggctggtttctcctgagactgcatcaacgtcaacgatctcaccaggccaacccagcagcacatcttacaggagccaacgccagcttgataggtattggtggacctctgttatgggagacacgacttggcctggccttcttgaggggcttgacgtatcatgatggggccctggtgaccatggagcccggttactactatgtgtactccaaagtgcagctgagcggcgtgggctgcccccaggggctggccaatggcctccccatcacccatggactatacaagcgcacatcccgctacccgaaggagttagaactgctggtcagtcggcggtcaccctgtggccgggccaacagctcccgagtctggtgggacagcagcttcctgggcggcgtggtacatctggaggctggggaagaggtggtggtccgcgtgcctggaaaccgcctggtcagaccacgtgacggcaccaggtcctatttcggagctttcatggtctga

SEQ ID NO: 4 - DNA, 1872 bp, synthetic construct
  source 1..1872: mol_type = other DNA
atggcgcccgccgccctctgggtcgcgctggtcttcgaactgcagctgtgggccaccgggcacacagtgcccgcccaggttgtcttgacaccctacaaaccggaacctgggtacgagtgccagatctcacaggaatactatgacaggaaggctcagatgtgctgtgctaagtgtcctcctggccaatatgtgaaacatttctgcaacaagacctcggacaccgtgtgtgcggactgtgaggcaagcatgtatacccaggtctggaaccagtttcgtacatgtttgagctgcagttcttcctgtaccactgaccaggtggagatccgcgcctgcactaaacagcagaaccgagtgtgtgcttgcgaagctggcaggtactgcgccttgaaaacccattctggcagctgtcgacagtgcatgaggctgagcaagtgcggccctggcttcggagtggccagttcaagagccccaaatggaaatgtgctatgcaaggcctgtgccccagggacgttctctgacaccacatcatccactgatgtgtgcaggccccaccgcatctgtagcatcctggctattcccggaaatgcaagcacagatgcagtctgtgcgcccgagtccccaactctaagtgccatcccaaggacactctacgtatctcagccagagcccacaagatcccaacccctggatcaagagccagggcccagccaaactccaagcatccttacatcgttgggttcaacccccattattgaacaaagtaccaagggtggcatctctcttccaattggtctgattgttggagtgacatcactgggtctgctgatgttaggactggtgaactgcatcatcctggtgcagaggaacagcagaagaaacagaggcggccagagcgactacatgaacatgacccccagaaggccaggcctgaccagaaagccctaccagccctacgcccctgccagagacttcgccgcctacagacccagagccaagttcagcagatccgccgagacagccgccaacctgcaggatcccaaccagctgtacaacgagctgaacctgggcagacgggaggaatacgacgtgctggaaaagaagagagccagggaccccgagatgggcggcaagcagcagagaagaagaaaccctcaggaaggcgtctacaacgccctgcagaaagacaagatggccgaggcctacagcgagatcggcaccaagggcgagagaagaaggggcaagggccacgatggcctgtaccagggcctgtccaccgccaccaaggacacctacgacgccctgcacatgcagaccctggcccccagagccacgaacttctctctgttaaagcaagcaggagacgtggaagaaaaccccggtcctatgaacccagccatcagcgtcgctctcctgctctcagtcttgcaggtgtcccgagggcagaaggtgaccagcctgacagcctgcctggtgaaccaaaaccttcgcctggactgccgccatgagaataacaccaaggataactccatccagcatgagttcagcctgacccgagagaagaggaagcacgtgctctcaggcacccttgggatacccgagcacacgtaccgctcccgcgtcaccctctccaaccagccctatatcaaggtccttaccctagccaacttcaccaccaaggatgagggcgactacttttgtgagcttcgcgtctcgggcgcgaatcccatgagctccaataaaagtatcagtgtgtatagagacaagctggtcaagtgtggcggcataagcctgctggttcagaacacatcctggatgctgctgctgctgctttccctctccctcctccaagccctggacttcatttctctgtga

SEQ ID NO: 5 - protein, 623 aa, synthetic construct
MAPAALWVALVFELQLWATGHTVPAQVVLTPYKPEPGYECQISQEYYDRKAQMCCAKCPPGQYVKHFCNKTSDTVCADCEASMYTQVWNQFRTCLSCSSSCTTDQVEIRACTKQQNRVCACEAGRYCALKTHSGSCRQCMRLSKCGPGFGVASSRAPNGNVLCKACAPGTFSDTTSSTDVCRPHRICSILAIPGNASTDAVCAPESPTLSAIPRTLYVSQPEPTRSQPLDQEPGPSQTPSILTSLGSTPIIEQSTKGGISLPIGLIVGVTSLGLLMLGLVNCIILVQRNSRRNRGGQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRATNFSLLKQAGDVEENPGPMNPAISVALLLSVLQVSRGQKVTSLTACLVNQNLRLDCRHENNTKDNSIQHEFSLTREKRKHVLSGTLGIPEHTYRSRVTLSNQPYIKVLTLANFTTKDEGDYFCELRVSGANPMSSNKSISVYRDKLVKCGGISLLVQNTSWMLLLLLSLSLLQALDFISL

SEQ ID NO: 6 - DNA, 864 bp, Mus musculus
  source 1..864: mol_type = genomic DNA
atggcgcccgccgccctctgggtcgcgctggtcttcgaactgcagctgtgggccaccgggcacacagtgcccgcccaggttgtcttgacaccctacaaaccggaacctgggtacgagtgccagatctcacaggaatactatgacaggaaggctcagatgtgctgtgctaagtgtcctcctggccaatatgtgaaacatttctgcaacaagacctcggacaccgtgtgtgcggactgtgaggcaagcatgtatacccaggtctggaaccagtttcgtacatgtttgagctgcagttcttcctgtaccactgaccaggtggagatccgcgcctgcactaaacagcagaaccgagtgtgtgcttgcgaagctggcaggtactgcgccttgaaaacccattctggcagctgtcgacagtgcatgaggctgagcaagtgcggccctggcttcggagtggccagttcaagagccccaaatggaaatgtgctatgcaaggcctgtgccccagggacgttctctgacaccacatcatccactgatgtgtgcaggccccaccgcatctgtagcatcctggctattcccggaaatgcaagcacagatgcagtctgtgcgcccgagtccccaactctaagtgccatcccaaggacactctacgtatctcagccagagcccacaagatcccaacccctggatcaagagccagggcccagccaaactccaagcatccttacatcgttgggttcaacccccattattgaacaaagtaccaagggtggcatctctcttccaattggtctgattgttggagtgacatcactgggtctgctgatgttaggactggtgaactgcatcatcctggtgcagagg

SEQ ID NO: 7 - protein, 288 aa, Mus musculus
MAPAALWVALVFELQLWATGHTVPAQVVLTPYKPEPGYECQISQEYYDRKAQMCCAKCPPGQYVKHFCNKTSDTVCADCEASMYTQVWNQFRTCLSCSSSCTTDQVEIRACTKQQNRVCACEAGRYCALKTHSGSCRQCMRLSKCGPGFGVASSRAPNGNVLCKACAPGTFSDTTSSTDVCRPHRICSILAIPGNASTDAVCAPESPTLSAIPRTLYVSQPEPTRSQPLDQEPGPSQTPSILTSLGSTPIIEQSTKGGISLPIGLIVGVTSLGLLMLGLVNCIILVQR

SEQ ID NO: 8 - DNA, 123 bp, Mus musculus
  source 1..123: mol_type = genomic DNA
aacagcagaagaaacagaggcggccagagcgactacatgaacatgacccccagaaggccaggcctgaccagaaagccctaccagccctacgcccctgccagagacttcgccgcctacagaccc

SEQ ID NO: 9 - protein, 41 aa, Mus musculus
NSRRNRGGQSDYMNMTPRRPGLTRKPYQPYAPARDFAAYRP

SEQ ID NO: 10 - DNA, 339 bp, Mus musculus
  source 1..339: mol_type = genomic DNA
agagccaagttcagcagatccgccgagacagccgccaacctgcaggatcccaaccagctgtacaacgagctgaacctgggcagacgggaggaatacgacgtgctggaaaagaagagagccagggaccccgagatgggcggcaagcagcagagaagaagaaaccctcaggaaggcgtctacaacgccctgcagaaagacaagatggccgaggcctacagcgagatcggcaccaagggcgagagaagaaggggcaagggccacgatggcctgtaccagggcctgtccaccgccaccaaggacacctacgacgccctgcacatgcagaccctggcccccaga

SEQ ID NO: 11 - protein, 113 aa, Mus musculus
RAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 12 - DNA, 57 bp, synthetic construct
  source 1..57: mol_type = other DNA
gccacgaacttctctctgttaaagcaagcaggagacgtggaagaaaaccccggtcct

SEQ ID NO: 13 - protein, 19 aa, synthetic construct
ATNFSLLKQAGDVEENPGP

SEQ ID NO: 14 - DNA, 489 bp, Mus musculus
  source 1..489: mol_type = genomic DNA
atgaacccagccatcagcgtcgctctcctgctctcagtcttgcaggtgtcccgagggcagaaggtgaccagcctgacagcctgcctggtgaaccaaaaccttcgcctggactgccgccatgagaataacaccaaggataactccatccagcatgagttcagcctgacccgagagaagaggaagcacgtgctctcaggcacccttgggatacccgagcacacgtaccgctcccgcgtcaccctctccaaccagccctatatcaaggtccttaccctagccaacttcaccaccaaggatgagggcgactacttttgtgagcttcgcgtctcgggcgcgaatcccatgagctccaataaaagtatcagtgtgtatagagacaagctggtcaagtgtggcggcataagcctgctggttcagaacacatcctggatgctgctgctgctgctttccctctccctcctccaagccctggacttcatttctctgtga

SEQ ID NO: 15 - protein, 162 aa, Mus musculus
MNPAISVALLLSVLQVSRGQKVTSLTACLVNQNLRLDCRHENNTKDNSIQHEFSLTREKRKHVLSGTLGIPEHTYRSRVTLSNQPYIKVLTLANFTTKDEGDYFCELRVSGANPMSSNKSISVYRDKLVKCGGISLLVQNTSWMLLLLLSLSLLQALDFISL

SEQ ID NO: 16 - DNA, 1749 bp, synthetic construct
  source 1..1749: mol_type = other DNA
atgcgcctgccccgggcctcctctccctgcggcctggcctgggggccactcctgctgggactcagcgggcttctggtggcctctcagccccagctggtgcccccttatcgcatagaaaaccagacttgctgggaccaggacaaggaatactacgagcccatgcacgacgtctgctgctcccgctgtcccccaggcgagtttgtctttgcggtatgcagccgcagccaagacacggtttgcaagacttgcccccataattcctataatgaacactggaaccatctctccacctgccagctgtgccgcccctgtgacattgtgctgggctttgaggaggttgccccttgcaccagcgatcggaaagccgagtgccgctgtcagccggggatgtcctgtgtgtatctggacaatgagtgtgtgcactgtgaggaggagcggcttgtactctgccagcctggcacagaagccgaggtcacagatgaaattatggatactgacgtcaactgtgtcccctgtaagccgggacacttccagaacacttcctcccctcgagcccgctgtcaaccccataccagatgtgagatccagggcctggtggaggcagctccaggtacctcctactcggataccatctgtaaaaatcccccagagccaggagcaatgctactgctggccatcctgctgtcgctggtcctctttctgctcttcaccactgtcctggcctgtgcctgggcggccgcgaacagcagaagaaacagaggcggccagagcgactacatgaacatgacccccagaaggccaggcctgaccagaaagccctaccagccctacgcccctgccagagacttcgccgcctacagacccagagccaagttcagcagatccgccgagacagccgccaacctgcaggatcccaaccagctgtacaacgagctgaacctgggcagacgggaggaatacgacgtgctggaaaagaagagagccagggaccccgagatgggcggcaagcagcagagaagaagaaaccctcaggaaggcgtctacaacgccctgcagaaagacaagatggccgaggcctacagcgagatcggcaccaagggcgagagaagaaggggcaagggccacgatggcctgtaccagggcctgtccaccgccaccaaggacacctacgacgccctgcacatgcagaccctggcccccagagccacgaacttctctctgttaaagcaagcaggagacgtggaagaaaaccccggtcctatgaacccagccatcagcgtcgctctcctgctctcagtcttgcaggtgtcccgagggcagaaggtgaccagcctgacagcctgcctggtgaaccaaaaccttcgcctggactgccgccatgagaataacaccaaggataactccatccagcatgagttcagcctgacccgagagaagaggaagcacgtgctctcaggcacccttgggatacccgagcacacgtaccgctcccgcgtcaccctctccaaccagccctatatcaaggtccttaccctagccaacttcaccaccaaggatgagggcgactacttttgtgagcttcgcgtctcgggcgcgaatcccatgagctccaataaaagtatcagtgtgtatagagacaagctggtcaagtgtggcggcataagcctgctggttcagaacacatcctggatgctgctgctgctgctttccctctccctcctccaagccctggacttcatttctctgtga

SEQ ID NO: 17 - protein, 582 aa, synthetic construct
MRLPRASSPCGLAWGPLLLGLSGLLVASQPQLVPPYRIENQTCWDQDKEYYEPMHDVCCSRCPPGEFVFAVCSRSQDTVCKTCPHNSYNEHWNHLSTCQLCRPCDIVLGFEEVAPCTSDRKAECRCQPGMSCVYLDNECVHCEEERLVLCQPGTEAEVTDEIMDTDVNCVPCKPGHFQNTSSPRARCQPHTRCEIQGLVEAAPGTSYSDTICKNPPEPGAMLLLAILLSLVLFLLFTTVLACAWAAANSRRNRGGQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRATNFSLLKQAGDVEENPGPMNPAISVALLLSVLQVSRGQKVTSLTACLVNQNLRLDCRHENNTKDNSIQHEFSLTREKRKHVLSGTLGIPEHTYRSRVTLSNQPYIKVLTLANFTTKDEGDYFCELRVSGANPMSSNKSISVYRDKLVKCGGISLLVQNTSWMLLLLLSLSLLQALDFISL

SEQ ID NO: 18 - DNA, 741 bp, Mus musculus
  source 1..741: mol_type = genomic DNA
atgcgcctgccccgggcctcctctccctgcggcctggcctgggggccactcctgctgggactcagcgggcttctggtggcctctcagccccagctggtgcccccttatcgcatagaaaaccagacttgctgggaccaggacaaggaatactacgagcccatgcacgacgtctgctgctcccgctgtcccccaggcgagtttgtctttgcggtatgcagccgcagccaagacacggtttgcaagacttgcccccataattcctataatgaacactggaaccatctctccacctgccagctgtgccgcccctgtgacattgtgctgggctttgaggaggttgccccttgcaccagcgatcggaaagccgagtgccgctgtcagccggggatgtcctgtgtgtatctggacaatgagtgtgtgcactgtgaggaggagcggcttgtactctgccagcctggcacagaagccgaggtcacagatgaaattatggatactgacgtcaactgtgtcccctgtaagccgggacacttccagaacacttcctcccctcgagcccgctgtcaaccccataccagatgtgagatccagggcctggtggaggcagctccaggtacctcctactcggataccatctgtaaaaatcccccagagccaggagcaatgctactgctggccatcctgctgtcgctggtcctctttctgctcttcaccactgtcctggcctgtgcctgggcggccgcg

SEQ ID NO: 19 - protein, 247 aa, Mus musculus
MRLPRASSPCGLAWGPLLLGLSGLLVASQPQLVPPYRIENQTCWDQDKEYYEPMHDVCCSRCPPGEFVFAVCSRSQDTVCKTCPHNSYNEHWNHLSTCQLCRPCDIVLGFEEVAPCTSDRKAECRCQPGMSCVYLDNECVHCEEERLVLCQPGTEAEVTDEIMDTDVNCVPCKPGHFQNTSSPRARCQPHTRCEIQGLVEAAPGTSYSDTICKNPPEPGAMLLLAILLSLVLFLLFTTVLACAWAAA

SEQ ID NO: 20 - DNA, 1662 bp, synthetic construct
  source 1..1662: mol_type = other DNA
atggaggcacggctgctgcggggctgcgtggtggagcctctgttcctaccactgctgctgctgctgctgctgctgcttggtggccagggccagggcggcatgtctggcaggtgtgactgtgccagtgagtcccagaagaggtatggcccgttttgttgcaggggctgcccaaagggacactacatgaaggccccctgcgcagaaccctgtggcaactccacctgccttccctgtccctcggacaccttcttgaccagagacaaccactttaagactgactgtacccgctgccaagtctgtgatgaagaggcccttcaagtgacccttgagaactgctcggcaaagtcggacacccactgtggctgccagtcaggctggtgtgttgactgctccaccgagccatgtgggaaaagctcacctttctcttgtgtcccatgcggggctacaacaccagtccatgaggctccaaccccccggccctgcctgcctggcttctatatacgtggcaatgactgcacgtcctgccccacgggcttcagcagcgtttgccctaaggcttgcactgctgtctgtggctggaagcagatgttttgggtccaggtgcttctaggagtcgcgttcctttttggggctatcctgatctgtgcatattgtaacagcagaagaaacagaggcggccagagcgactacatgaacatgacccccagaaggccaggcctgaccagaaagccctaccagccctacgcccctgccagagacttcgccgcctacagacccagagccaagttcagcagatccgccgagacagccgccaacctgcaggatcccaaccagctgtacaacgagctgaacctgggcagacgggaggaatacgacgtgctggaaaagaagagagccagggaccccgagatgggcggcaagcagcagagaagaagaaaccctcaggaaggcgtctacaacgccctgcagaaagacaagatggccgaggcctacagcgagatcggcaccaagggcgagagaagaaggggcaagggccacgatggcctgtaccagggcctgtccaccgccaccaaggacacctacgacgccctgcacatgcagaccctggcccccagagccacgaacttctctctgttaaagcaagcaggagacgtggaagaaaaccccggtcctatgaacccagccatcagcgtcgctctcctgctctcagtcttgcaggtgtcccgagggcagaaggtgaccagcctgacagcctgcctggtgaaccaaaaccttcgcctggactgccgccatgagaataacaccaaggataactccatccagcatgagttcagcctgacccgagagaagaggaagcacgtgctctcaggcacccttgggatacccgagcacacgtaccgctcccgcgtcaccctctccaaccagccctatatcaaggtccttaccctagccaacttcaccaccaaggatgagggcgactacttttgtgagcttcgcgtctcgggcgcgaatcccatgagctccaataaaagtatcagtgtgtatagagacaagctggtcaagtgtggcggcataagcctgctggttcagaacacatcctggatgctgctgctgctgctttccctctccctcctccaagccctggacttcatttctctgtga

SEQ ID NO: 21 - protein, 553 aa, synthetic construct
MEARLLRGCVVEPLFLPLLLLLLLLLGGQGQGGMSGRCDCASESQKRYGPFCCRGCPKGHYMKAPCAEPCGNSTCLPCPSDTFLTRDNHFKTDCTRCQVCDEEALQVTLENCSAKSDTHCGCQSGWCVDCSTEPCGKSSPFSCVPCGATTPVHEAPTPRPCLPGFYIRGNDCTSCPTGFSSVCPKACTAVCGWKQMFWVQVLLGVAFLFGAILICAYCNSRRNRGGQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRATNFSLLKQAGDVEENPGPMNPAISVALLLSVLQVSRGQKVTSLTACLVNQNLRLDCRHENNTKDNSIQHEFSLTREKRKHVLSGTLGIPEHTYRSRVTLSNQPYIKVLTLANFTTKDEGDYFCELRVSGANPMSSNKSISVYRDKLVKCGGISLLVQNTSWMLLLLLSLSLLQALDFISL

SEQ ID NO: 22 - DNA, 654 bp, Mus musculus
  source 1..654: mol_type = genomic DNA
atggaggcacggctgctgcggggctgcgtggtggagcctctgttcctaccactgctgctgctgctgctgctgctgcttggtggccagggccagggcggcatgtctggcaggtgtgactgtgccagtgagtcccagaagaggtatggcccgttttgttgcaggggctgcccaaagggacactacatgaaggccccctgcgcagaaccctgtggcaactccacctgccttccctgtccctcggacaccttcttgaccagagacaaccactttaagactgactgtacccgctgccaagtctgtgatgaagaggcccttcaagtgacccttgagaactgctcggcaaagtcggacacccactgtggctgccagtcaggctggtgtgttgactgctccaccgagccatgtgggaaaagctcacctttctcttgtgtcccatgcggggctacaacaccagtccatgaggctccaaccccccggccctgcctgcctggcttctatatacgtggcaatgactgcacgtcctgccccacgggcttcagcagcgtttgccctaaggcttgcactgctgtctgtggctggaagcagatgttttgggtccaggtgcttctaggagtcgcgttcctttttggggctatcctgatctgtgcatattgt

SEQ ID NO: 23 - protein, 218 aa, Mus musculus
MEARLLRGCVVEPLFLPLLLLLLLLLGGQGQGGMSGRCDCASESQKRYGPFCCRGCPKGHYMKAPCAEPCGNSTCLPCPSDTFLTRDNHFKTDCTRCQVCDEEALQVTLENCSAKSDTHCGCQSGWCVDCSTEPCGKSSPFSCVPCGATTPVHEAPTPRPCLPGFYIRGNDCTSCPTGFSSVCPKACTAVCGWKQMFWVQVLLGVAFLFGAILICAYC

SEQ ID NO: 24 - DNA, 1155 bp, synthetic construct
  source 1..1155: mol_type = other DNA
atggcgcccgccgccctctgggtcgcgctggtcttcgaactgcagctgtgggccaccgggcacacattctgggccctggtggtggtggccggcgtgctgttctgttacggcctgctggtcacagtggccctgtgcgtgatctggaccaacagcagaagaaacagaggcggccagagcgactacatgaacatgacccccagaaggccaggcctgaccagaaagccctaccagccctacgcccctgccagagacttcgccgcctacagacccagagccaagttcagcagatccgccgagacagccgccaacctgcaggatcccaaccagctgtacaacgagctgaacctgggcagacgggaggaatacgacgtgctggaaaagaagagagccagggaccccgagatgggcggcaagcagcagagaagaagaaaccctcaggaaggcgtctacaacgccctgcagaaagacaagatggccgaggcctacagcgagatcggcaccaagggcgagagaagaaggggcaagggccacgatggcctgtaccagggcctgtccaccgccaccaaggacacctacgacgccctgcacatgcagaccctggcccccagagccacgaacttctctctgttaaagcaagcaggagacgtggaagaaaaccccggtcctatgaacccagccatcagcgtcgctctcctgctctcagtcttgcaggtgtcccgagggcagaaggtgaccagcctgacagcctgcctggtgaaccaaaaccttcgcctggactgccgccatgagaataacaccaaggataactccatccagcatgagttcagcctgacccgagagaagaggaagcacgtgctctcaggcacccttgggatacccgagcacacgtaccgctcccgcgtcaccctctccaaccagccctatatcaaggtccttaccctagccaacttcaccaccaaggatgagggcgactacttttgtgagcttcgcgtctcgggcgcgaatcccatgagctccaataaaagtatcagtgtgtatagagacaagctggtcaagtgtggcggcataagcctgctggttcagaacacatcctggatgctgctgctgctgctttccctctccctcctccaagccctggacttcatttctctgtga

SEQ ID NO: 25 - protein, 384 aa, synthetic construct
MAPAALWVALVFELQLWATGHTFWALVVVAGVLFCYGLLVTVALCVIWTNSRRNRGGQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPRATNFSLLKQAGDVEENPGPMNPAISVALLLSVLQVSRGQKVTSLTACLVNQNLRLDCRHENNTKDNSIQHEFSLTREKRKHVLSGTLGIPEHTYRSRVTLSNQPYIKVLTLANFTTKDEGDYFCELRVSGANPMSSNKSISVYRDKLVKCGGISLLVQNTSWMLLLLLSLSLLQALDFISL

SEQ ID NO: 26 - DNA, 66 bp, Mus musculus
  source 1..66: mol_type = genomic DNA
atggcgcccgccgccctctgggtcgcgctggtcttcgaactgcagctgtgggccaccgggcacaca

SEQ ID NO: 27 - protein, 22 aa, Mus musculus
MAPAALWVALVFELQLWATGHT

SEQ ID NO: 28 - DNA, 81 bp, Mus musculus
  source 1..81: mol_type = genomic DNA
ttctgggccctggtggtggtggccggcgtgctgttctgttacggcctgctggtcacagtggccctgtgcgtgatctggacc

SEQ ID NO: 29 - protein, 27 aa, Mus musculus
FWALVVVAGVLFCYGLLVTVALCVIWT

SEQ ID NO: 30 - DNA, 1752 bp, synthetic construct
  source 1..1752: mol_type = other DNA
atgctcctgccttgggccacctctgcccccggcctggcctgggggcctctggtgctgggcctcttcgggctcctggcagcatcgcagccccaggcggtgcctccatatgcgtcggagaaccagacctgcagggaccaggaaaaggaatactatgagccccagcaccgcatctgctgctcccgctgcccgccaggcacctatgtctcagctaaatgtagccgcatccgggacacagtttgtgccacatgtgccgagaattcctacaacgagcactggaactacctgaccatctgccagctgtgccgcccctgtgacccagtgatgggcctcgaggagattgccccctgcacaagcaaacggaagacccagtgccgctgccagccgggaatgttctgtgctgcctgggccctcgagtgtacacactgcgagctactttctgactgcccgcctggcactgaagccgagctcaaagatgaagttgggaagggtaacaaccactgcgtcccctgcaaggccgggcacttccagaatacctcctcccccagcgcccgctgccagccccacaccaggtgtgagaaccaaggtctggtggaggcagctccaggcactgcccagtccgacacaacctgcaaaaatccattagagccactgcccccagagatgtcaggaaccatgctgatgctggccgttctgctgccactggccttctttctgctccttgccaccgtcttctcctgcatctggaggagtaagaggagcaggctcctgcacagtgactacatgaacatgactccccgccgccccgggcccacccgcaagcattaccagccctatgcccccccacgcgacttcgcagcctatcgctccctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgccacgaacttctctctgttaaagcaagcaggagacgtggaagaaaaccccggtcctatgaacccagccatcagcgtcgctctcctgctctcagtcttgcaggtgtcccgagggcagaaggtgaccagcctgacagcctgcctggtgaaccaaaaccttcgcctggactgccgccatgagaataacaccaaggataactccatccagcatgagttcagcctgacccgagagaagaggaagcacgtgctctcaggcacccttgggatacccgagcacacgtaccgctcccgcgtcaccctctccaaccagccctatatcaaggtccttaccctagccaacttcaccaccaaggatgagggcgactacttttgtgagcttcgcgtctcgggcgcgaatcccatgagctccaataaaagtatcagtgtgtatagagacaagctggtcaagtgtggcggcataagcctgctggttcagaacacatcctggatgctgctgctgctgctttccctctccctcctccaagccctggacttcatttctctgtga

SEQ ID NO: 31 - protein, 583 aa, Homo sapiens
MLLPWATSAPGLAWGPLVLGLFGLLAASQPQAVPPYASENQTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKNPLEPLPPEMSGTMLMLAVLLPLAFFLLLATVFSCIWRSKRSRLLHSDYMNMTPRRPGPTRKHYQPYAPPRDFAAYRSLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRATNFSLLKQAGDVEENPGPMNPAISVALLLSVLQVSRGQKVTSLTACLVNQNLRLDCRHENNTKDNSIQHEFSLTREKRKHVLSGTLGIPEHTYRSRVTLSNQPYIKVLTLANFTTKDEGDYFCELRVSGANPMSSNKSISVYRDKLVKCGGISLLVQNTSWMLLLLLSLSLLQALDFISL

SEQ ID NO: 32 - DNA, 744 bp, Homo sapiens
  source 1..744: mol_type = genomic DNA
atgctcctgccttgggccacctctgcccccggcctggcctgggggcctctggtgctgggcctcttcgggctcctggcagcatcgcagccccaggcggtgcctccatatgcgtcggagaaccagacctgcagggaccaggaaaaggaatactatgagccccagcaccgcatctgctgctcccgctgcccgccaggcacctatgtctcagctaaatgtagccgcatccgggacacagtttgtgccacatgtgccgagaattcctacaacgagcactggaactacctgaccatctgccagctgtgccgcccctgtgacccagtgatgggcctcgaggagattgccccctgcacaagcaaacggaagacccagtgccgctgccagccgggaatgttctgtgctgcctgggccctcgagtgtacacactgcgagctactttctgactgcccgcctggcactgaagccgagctcaaagatgaagttgggaagggtaacaaccactgcgtcccctgcaaggccgggcacttccagaatacctcctcccccagcgcccgctgccagccccacaccaggtgtgagaaccaaggtctggtggaggcagctccaggcactgcccagtccgacacaacctgcaaaaatccattagagccactgcccccagagatgtcaggaaccatgctgatgctggccgttctgctgccactggccttctttctgctccttgccaccgtcttctcctgcatctgg

SEQ ID NO: 33 - protein, 248 aa, Homo sapiens
MLLPWATSAPGLAWGPLVLGLFGLLAASQPQAVPPYASENQTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKNPLEPLPPEMSGTMLMLAVLLPLAFFLLLATVFSCIW

SEQ ID NO: 34 - DNA, 123 bp, Homo sapiens
  source 1..123: mol_type = genomic DNA
aggagtaagaggagcaggctcctgcacagtgactacatgaacatgactccccgccgccccgggcccacccgcaagcattaccagccctatgcccccccacgcgacttcgcagcctatcgctcc

SEQ ID NO: 35 - protein, 41 aa, Homo sapiens
RSKRSRLLHSDYMNMTPRRPGPTRKHYQPYAPPRDFAAYRS

SEQ ID NO: 36 - DNA, 339 bp, Homo sapiens
  source 1..339: mol_type = genomic DNA
ctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 37 - protein, 113 aa, Homo sapiens
LRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 38 - protein, 441 aa, synthetic construct
MAPVAVWAALAVGLELWAAAHALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDFALPVGLIVGVTALGLLIIGVVNCVIMTQVRSKRSRLLHSDYMNMTPRRPGPTRKHYQPYAPPRDFAAYRSLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 39 - protein, 287 aa, Homo sapiens
MAPVAVWAALAVGLELWAAAHALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDFALPVGLIVGVTALGLLIIGVVNCVIMTQV

SEQ ID NO: 40 - protein, 374 aa, synthetic construct
MEQRPRGCAAVAAALLLVLLGARAQGGTRSPRCDCAGDFHKKIGLFCCRGCPAGHYLKAPCTEPCGNSTCLVCPQDTFLAWENHHNSECARCQACDEQASQVALENCSAVADTRCGCKPGWFVECQVSQCVSSSPFYCQPCLDCGALHRHTRLLCSRRDTDCGTCLPGFYEHGDGCVSCPTSTLGSCPERCAAVCGWRQMFWVQVLLAGLVVPLLLGATLRSKRSRLLHSDYMNMTPRRPGPTRKHYQPYAPPRDFAAYRSLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 41 - protein, 220 aa, Homo sapiens
MEQRPRGCAAVAAALLLVLLGARAQGGTRSPRCDCAGDFHKKIGLFCCRGCPAGHYLKAPCTEPCGNSTCLVCPQDTFLAWENHHNSECARCQACDEQASQVALENCSAVADTRCGCKPGWFVECQVSQCVSSSPFYCQPCLDCGALHRHTRLLCSRRDTDCGTCLPGFYEHGDGCVSCPTSTLGSCPERCAAVCGWRQMFWVQVLLAGLVVPLLLGATL

SEQ ID NO: 42 - DNA, 1107 bp, synthetic construct
  source 1..1107: mol_type = other DNA
atggtgtctttgcctcggctgtgcgcgctatggggctgcttgttgacagcggtccatctagggcagtgtgttacgtgcagtgacaaacagtacctccacgatggccagtgctgtgatttgtgccagccaggaagccgactgacaagccactgcacagctcttgagaagacccaatgccacccatgtgactcaggcgaattctcagcccagtggaacagggagattcgctgtcaccagcacagacactgtgaacccaatcaagggcttcgggttaagaaggagggcaccgcagaatcagacactgtctgtacctgtaaggaaggacaacactgcaccagcaaggattgcgaggcatgtgctcagcacacgccctgtatccctggctttggagttatggagatggccactgagaccactgataccgtctgtcatccctgcccagtcggcttcttctccaatcagtcatcacttttcgaaaagtgttatccctggacaagctgtgaggataagaacttggaggtcctacagaaaggaacgagtcagactaatgtcatctgtggtttaaagtcccggatgcgagccctgctggtcattcctgtcgtgatgggcatcctcatcaccattttcggggtgtttctctatatcaacagcagaagaaacagaggcggccagagcgactacatgaacatgacccccagaaggccaggcctgaccagaaagccctaccagccctacgcccctgccagagacttcgccgcctacagacccagagccaagttcagcagatccgccgagacagccgccaacctgcaggatcccaaccagctgtacaacgagctgaacctgggcagacgggaggaatacgacgtgctggaaaagaagagagccagggaccccgagatgggcggcaagcagcagagaagaagaaaccctcaggaaggcgtctacaacgccctgcagaaagacaagatggccgaggcctacagcgagatcggcaccaagggcgagagaagaaggggcaagggccacgatggcctgtaccagggcctgtccaccgccaccaaggacacctacgacgccctgcacatgcagaccctggcccccaga

SEQ ID NO: 43 - protein, 369 aa, synthetic construct
MVSLPRLCALWGCLLTAVHLGQCVTCSDKQYLHDGQCCDLCQPGSRLTSHCTALEKTQCHPCDSGEFSAQWNREIRCHQHRHCEPNQGLRVKKEGTAESDTVCTCKEGQHCTSKDCEACAQHTPCIPGFGVMEMATETTDTVCHPCPVGFFSNQSSLFEKCYPWTSCEDKNLEVLQKGTSQTNVICGLKSRMRALLVIPVVMGILITIFGVFLYINSRRNRGGQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 44 - DNA, 645 bp, Mus musculus
  source 1..645: mol_type = genomic DNA
atggtgtctttgcctcggctgtgcgcgctatggggctgcttgttgacagcggtccatctagggcagtgtgttacgtgcagtgacaaacagtacctccacgatggccagtgctgtgatttgtgccagccaggaagccgactgacaagccactgcacagctcttgagaagacccaatgccacccatgtgactcaggcgaattctcagcccagtggaacagggagattcgctgtcaccagcacagacactgtgaacccaatcaagggcttcgggttaagaaggagggcaccgcagaatcagacactgtctgtacctgtaaggaaggacaacactgcaccagcaaggattgcgaggcatgtgctcagcacacgccctgtatccctggctttggagttatggagatggccactgagaccactgataccgtctgtcatccctgcccagtcggcttcttctccaatcagtcatcacttttcgaaaagtgttatccctggacaagctgtgaggataagaacttggaggtcctacagaaaggaacgagtcagactaatgtcatctgtggtttaaagtcccggatgcgagccctgctggtcattcctgtcgtgatgggcatcctcatcaccattttcggggtgtttctctatatc

SEQ ID NO: 45 - protein, 215 aa, Mus musculus
MVSLPRLCALWGCLLTAVHLGQCVTCSDKQYLHDGQCCDLCQPGSRLTSHCTALEKTQCHPCDSGEFSAQWNREIRCHQHRHCEPNQGLRVKKEGTAESDTVCTCKEGQHCTSKDCEACAQHTPCIPGFGVMEMATETTDTVCHPCPVGFFSNQSSLFEKCYPWTSCEDKNLEVLQKGTSQTNVICGLKSRMRALLVIPVVMGILITIFGVFLYI

SEQ ID NO: 46 - protein, 215 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFI

SEQ ID NO: 47 - protein, 369 aa, synthetic construct
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIRSKRSRLLHSDYMNMTPRRPGPTRKHYQPYAPPRDFAAYRSLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 48 - protein, 290 aa, synthetic construct
MRLPRASSPCGLAWGPLLLGLSGLLVASQPQLVPPYRIENQTCWDQDKEYYEPMHDVCCSRCPPGEFVFAVCSRSQDTVCKTCPHNSYNEHWNHLSTCQLCRPCDIVLGFEEVAPCTSDRKAECRCQPGMSCVYLDNECVHCEEERLVLCQPGTEAEVTDEIMDTDVNCVPCKPGHFQNTSSPRARCQPHTRCEIQGLVEAAPGTSYSDTICKNPPEPGAMLLLAILLSLVLFLLFTTVLACAWAAANSRRNRGGQSDYMNMTPRRPGLTRKPYQPYAPARDFAAYRPID

SEQ ID NO: 49 - DNA, 1089 bp, synthetic construct
  source 1..1089: mol_type = other DNA
atggtgtctttgcctcggctgtgcgcgctatggggctgcttgttgacagcggtccatctagggcagtgtgttacgtgcagtgacaaacagtacctccacgatggccagtgctgtgatttgtgccagccaggaagccgactgacaagccactgcacagctcttgagaagacccaatgccacccatgtgactcaggcgaattctcagcccagtggaacagggagattcgctgtcaccagcacagacactgtgaacccaatcaagggcttcgggttaagaaggagggcaccgcagaatcagacactgtctgtacctgtaaggaaggacaacactgcaccagcaaggattgcgaggcatgtgctcagcacacgccctgtatccctggctttggagttatggagatggccactgagaccactgataccgtctgtcatccctgcccagtcggcttcttctccaatcagtcatcacttttcgaaaagtgttatccctggacaagctgtgaggataagaacttggaggtcctacagaaaggaacgagtcagactaatgtcatctgtggtttaaagtcccggatgcgagccctgctggtcattcctgtcgtgatgggcatcctcatcaccattttcggggtgtttctctatatctcaaaaaagaaatacggatctagtgtgcatgaccctaatagtgaatacatgttcatggcggcagtcaacacaaacaaaaagtctagacttgcaggtgtgacctcaagagccaagttcagcagatccgccgagacagccgccaacctgcaggatcccaaccagctgtacaacgagctgaacctgggcagacgggaggaatacgacgtgctggaaaagaagagagccagggaccccgagatgggcggcaagcagcagagaagaagaaaccctcaggaaggcgtctacaacgccctgcagaaagacaagatggccgaggcctacagcgagatcggcaccaagggcgagagaagaaggggcaagggccacgatggcctgtaccagggcctgtccaccgccaccaaggacacctacgacgccctgcacatgcagaccctggcccccaga

SEQ ID NO: 50 - protein, 363 aa, synthetic construct
MVSLPRLCALWGCLLTAVHLGQCVTCSDKQYLHDGQCCDLCQPGSRLTSHCTALEKTQCHPCDSGEFSAQWNREIRCHQHRHCEPNQGLRVKKEGTAESDTVCTCKEGQHCTSKDCEACAQHTPCIPGFGVMEMATETTDTVCHPCPVGFFSNQSSLFEKCYPWTSCEDKNLEVLQKGTSQTNVICGLKSRMRALLVIPVVMGILITIFGVFLYISKKKYGSSVHDPNSEYMFMAAVNTNKKSRLAGVTSRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 51 - DNA, 1119 bp, synthetic construct
  source 1..1119: mol_type = other DNA
atggtgtctttgcctcggctgtgcgcgctatggggctgcttgttgacagcggtccatctagggcagtgtgttacgtgcagtgacaaacagtacctccacgatggccagtgctgtgatttgtgccagccaggaagccgactgacaagccactgcacagctcttgagaagacccaatgccacccatgtgactcaggcgaattctcagcccagtggaacagggagattcgctgtcaccagcacagacactgtgaacccaatcaagggcttcgggttaagaaggagggcaccgcagaatcagacactgtctgtacctgtaaggaaggacaacactgcaccagcaaggattgcgaggcatgtgctcagcacacgccctgtatccctggctttggagttatggagatggccactgagaccactgataccgtctgtcatccctgcccagtcggcttcttctccaatcagtcatcacttttcgaaaagtgttatccctggacaagctgtgaggataagaacttggaggtcctacagaaaggaacgagtcagactaatgtcatctgtggtttaaagtcccggatgcgagccctgctggtcattcctgtcgtgatgggcatcctcatcaccattttcggggtgtttctctatatcaaatggatcaggaaaaaattcccccacatattcaagcaaccatttaagaagaccactggagcagctcaagaggaagatgcttgtagctgccgatgtccacaggaagaagaaggaggaggaggaggctatgagctgagagccaagttcagcagatccgccgagacagccgccaacctgcaggatcccaaccagctgtacaacgagctgaacctgggcagacgggaggaatacgacgtgctggaaaagaagagagccagggaccccgagatgggcggcaagcagcagagaagaagaaaccctcaggaaggcgtctacaacgccctgcagaaagacaagatggccgaggcctacagcgagatcggcaccaagggcgagagaagaaggggcaagggccacgatggcctgtaccagggcctgtccaccgccaccaaggacacctacgacgccctgcacatgcagaccctggcccccaga

SEQ ID NO: 52 - protein, 373 aa, synthetic construct
MVSLPRLCALWGCLLTAVHLGQCVTCSDKQYLHDGQCCDLCQPGSRLTSHCTALEKTQCHPCDSGEFSAQWNREIRCHQHRHCEPNQGLRVKKEGTAESDTVCTCKEGQHCTSKDCEACAQHTPCIPGFGVMEMATETTDTVCHPCPVGFFSNQSSLFEKCYPWTSCEDKNLEVLQKGTSQTNVICGLKSRMRALLVIPVVMGILITIFGVFLYIKWIRKKFPHIFKQPFKKTTGAAQEEDACSCRCPQEEEGGGGGYELRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 53 - protein, 366 aa, synthetic construct
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFICWLTKKKYSSSVHDPNGEYMFMRAVNTAKKSRLTDVTLLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 54 - protein, 370 aa, synthetic construct
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKRGRKKLLYIFKQPFMRPVQTTQEEDGCSCRFPEEEEGGCELLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 55 - protein, 35 aa, Mus musculus
SKKKYGSSVHDPNSEYMFMAAVNTNKKSRLAGVTS

SEQ ID NO: 56 - protein, 45 aa, Mus musculus
KWIRKKFPHIFKQPFKKTTGAAQEEDACSCRCPQEEEGGGGGYEL

SEQ ID NO: 57 - protein, 38 aa, Homo sapiens
CWLTKKKYSSSVHDPNGEYMFMRAVNTAKKSRLTDVTL

SEQ ID NO: 58 - protein, 42 aa, Homo sapiens
KRGRKKLLYIFKQPFMRPVQTTQEEDGCSCRFPEEEEGGCEL